(19) United States
(12) Patent Application Publication
(10) Pub. No.: US 2024/0309371 A1
(43) Pub. Date: Sep. 19, 2024

(54) METHODS AND COMPOSITIONS TO DIAGNOSE AND TREAT TAUOPATHIES UTILIZING TRANSCRIPT VARIANTS OF PSEN2

(71) Applicant: University of Washington, Seattle, WA (US)

(72) Inventors: Paul Valdmanis, Seattle, WA (US); Meredith Course, Seattle, WA (US)

(73) Assignee: University of Washington, Seattle, WA (US)

(21) Appl. No.: 18/410,822

(22) Filed: Jan. 11, 2024

Related U.S. Application Data
(60) Provisional application No. 63/451,336, filed on Mar. 10, 2023.

(51) Int. Cl. C12N 15/113; A61P 25/28; C12Q 1/6883

(52) U.S. Cl. CPC C12N 15/113; A61P 25/28; C12Q 1/6883; C12N 2310/11; C12N 2310/14; C12N 2310/141; C12N 2310/3517; C12N 2310/531; C12Q 2600/156

(57) ABSTRACT

The current disclosure describes methods and composition for treating and diagnosing tauopathies utilizing transcript variants of PSEN2. Reduction of transcript variants of PSEN2 and/or preservation of canonical splicing can be beneficial in the treatment of tauopathies such as Alzheimer's disease. Methods of diagnosing sporadic Alzheimer's disease are also provided.

### CROSS-REFERENCE TO RELATED APPLICATION

This application claims priority to U.S. Provisional Patent Application No. 63/451,336 filed Mar. 10, 2023, which is incorporated herein by reference in its entirety as if fully set forth herein.

### STATEMENT REGARDING FEDERALLY SPONSORED RESEARCH OR DEVELOPMENT

This invention was made with government support under Grant No. P30 AG066509, awarded by the National Institute on Aging. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is W149-0027US.xml. The file is 195,599 bytes, was created on Jan. 10, 2024, and is being submitted electronically via Patent Center.

### FIELD OF THE DISCLOSURE

The current disclosure describes methods and composition for treating and diagnosing tauopathies utilizing transcript variants of PSEN2. Reduction of transcript variants of PSEN2 and/or preservation of canonical splicing can be beneficial in the treatment of tauopathies such as Alzheimer's disease. Methods of diagnosing tauopathies are also provided.

### BACKGROUND OF THE DISCLOSURE

Alzheimer disease (AD) is the most common neurodegenerative disease, characterized by dementia and premature death. Almost six million Americans have the disease—a number that will continue to escalate as the population ages—and still there remains no effective treatment.

Autosomal dominant forms of early-onset familial AD cases are caused in part by pathogenic variants in presenilin 1 (PSEN1) and presenilin 2 (PSEN2). Originally identified through linkage analyses, autosomal dominant mutations in PSEN1, PSEN2, and amyloid beta precursor protein (APP) lead to early-onset AD (onset <65 years old). These three risk genes are mechanistically united in the following way: either of the two presenilins form part of the γ-secretase complex, which cleaves APP to liberate a product called amyloid beta (Aβ) peptide. Though the means by which variants in PSEN1 and PSEN2 lead to AD are still debated, it is commonly thought that changes in PSEN1 or PSEN2 bias APP cleavage to form versions of Aβ peptide that are more prone to aggregation. Aggregation of Aβ, in turn, is a hallmark pathology of AD.

The proportion of early-onset familial AD cases attributed to variants in PSEN1 is 20%-70%, and those attributed to variants in PSEN2 is 5%. In addition to playing a role in early-onset AD, variants in both genes can also increase risk for or cause late-onset AD. It is generally thought that all disease-causing variants in these genes result in impairment of normal function of the γ-secretase complex, due to the resulting abnormal cleavage of APP and aggregation of Aβ. Tellingly, of all identified AD-related PSEN1 variants (there are now over 200), not one leads solely to a truncation or absence of protein product. PSEN2 variants are not as common as those in PSEN1 (19 identified so far), but they similarly do not appear to lead to haploinsufficiency. Despite these findings, there is a need to uncover the complex transcriptional changes taking place in AD to better develop targeted interventions.

### SUMMARY OF THE DISCLOSURE

The current disclosure describes methods and composition for treating and diagnosing tauopathies utilizing transcript variants of PSEN2. Reduction of transcript variants of PSEN2 and/or preservation of canonical splicing can be beneficial in the treatment of tauopathies such as Alzheimer's disease.

In particular embodiments, a method of treating or inhibiting tauopathies includes the administration of a reagent, such as an inhibitory nucleic acid molecule that targets a pathogenic transcript variant of PSEN2 thereby reducing the expression of the pathogenic transcript variant in a subject in need thereof. In particular embodiments, the tauopathy includes sporadic Alzheimer's disease. In particular embodiments, the tauopathy includes familial Alzheimer's disease or progressive supranuclear palsy.

In particular embodiments, the pathogenic transcript variants of PSEN2 include a transcript with the inclusion of exon 9B. In particular embodiments, exon 9B includes the sequence as set forth in SEQ ID NO: 2. In particular embodiments, the transcript with the inclusion of exon 9B includes the sequence as set forth in SEQ ID NO: 3, SEQ ID NO: 4, or SEQ ID NO: 5. In particular embodiments, the pathogenic transcript variant of PSEN2 include a transcript with the inclusion of 77 bp of the intronic sequence prior to exon 6. In particular embodiments, the 77 bp intronic sequence includes the sequence as set forth in SEQ ID NO: 6. In particular embodiments, the transcript with the inclusion of the 77 bp intronic sequence includes the sequence as set forth in SEQ ID NO: 7. In particular embodiments, a pathogenic transcript variant of PSEN2 is not wildtype PSEN2. In particular embodiments, wildtype PSEN2 includes the sequence as set forth in SEQ ID NO: 1.

In particular embodiments, the reagent (e.g., inhibitory nucleic acid molecule) that targets a pathogenic transcript variant of PSEN2 includes antisense oligonucleotides, short hairpin RNA (shRNA), small interfering RNA (siRNA), and/or microRNA (miRNA) that hybridize to a portion of the pathogenic transcript variant of PSEN2. In particular embodiments, a reagent that hybridizes to a portion of the pathogenic transcript variant of PSEN2 can have at least 80%, at least 85%, at least 90%, at least 95%, at least 98%, at least 99%, or 100% complementarity to the pathogenic transcript variant of PSEN2.

In particular embodiments, a method of treating or inhibiting tauopathies includes the administration of splice-switching oligonucleotides that target a pathogenic transcript variant of PSEN2. In particular embodiments, the splice-switching oligonucleotides hybridize to the pathogenic transcript variant of PSEN2 and shift splicing patterns thereby restoring the expression of wildtype PSEN2.

Methods of diagnosing tauopathies are also provided. In particular embodiments, a tauopathy includes sporadic Alzheimer's disease. In particular embodiments, a subject having a tauopathy has more transcript variants of PSEN2 compared to subjects without a neuropathologic disease. In particular embodiments, a method of diagnosing a subject includes detecting a level of one of more transcript variants of PSEN2 in a tissue sample of the subject. In particular embodiments, the level of transcript variants of PSEN2 will be higher in subjects with the tauopathy compared to a reference level. In particular embodiments, a method of diagnosing a subject with a tauopathy includes quantifying select alternative splicing isoforms of PSEN2 reads compared to total PSEN2 reads within a tissue sample of the subject. In particular embodiments, the proportion of select alternative splicing isoforms of PSEN2 will be higher in subjects with the tauopathy compared to a reference level. In particular embodiments, the reference level is the proportion of the level of transcript variants of PSEN2 or select alternative splicing isoforms of PSEN2 from tissue samples of subjects without a neuropathology.

### BRIEF DESCRIPTION OF THE FIGURES

Some of the drawings submitted herewith may be better understood in color. Applicant considers the color versions of the drawings as part of the original submission and reserves the right to present color images of the drawings in later proceedings.

FIG. 1. An example environment for administration of inhibitory nucleic acids that specifically bind pathogenic transcript variants of PSEN2.

FIGS. 2A, 2B. Iso-Seq reads mapping to PSEN1 and PSEN2. (2A) Schematic of probe location and number of probes used per gene. (2B) Iso-Seq reads mapping to PSEN1 and PSEN2. Reads that mapped to PSEN1 and PSEN2 were not significantly different across cohorts (P=0.62 for PSEN1 and P=0.53 for PSEN2, One-Way ANOVA). Bars are mean and SD. Sporadic Alzheimer's disease (AD) n=7, PSEN variant carrier n=7, control n=6.

FIGS. 3A-3H. Unique read analysis for PSEN1 and PSEN2 alternative splice products. (3A) Unique Iso-Seq reads mapping to PSEN1, PSEN2, and other regions in the genome. (3B) Ratio of 4 aa deletion to inclusion isoform of PSEN1. Abundance (percent out of total PSEN1 transcripts) of PSEN1 (3C) exon 3b inclusion isoform, and (3D) intron 8 retention isoform, across cohorts. (3E) Ratio of 1 aa deletion to inclusion isoform of PSEN2. Abundance (percent out of total PSEN2 transcripts) of PSEN2 (3F) exon 9B inclusion isoform, (3G) intron 77 inclusion isoform, and (3H) exon 6 skipping events, across cohorts. P-values were determined by a One-Way ANOVA, followed by Tukey's multiple comparisons. * <0.05, ** <0.01. All bars are mean and SD. Sporadic AD n=7, PSEN variant carrier n=7, control n=6.

FIGS. 4A, 4B. Iso-Seq reads not mapping to PSEN1 or PSEN2. (4A) Uniquely mapped reads that did not map to PSEN1 or PSEN2 were evenly distributed throughout the genome, providing no evidence of particular gene enrichment. The distribution of reads was also comparable between samples and cohorts. (4B) Total Iso-Seq reads (not including PSEN1 or PSEN2) did demonstrate enrichment for two genes on chromosome 18. From left to right, chromosomes are in order by number.

FIGS. 5A-5E. Isoforms of PSEN1. (5A) Schematic showing the three most predominant isoforms identified for PSEN1. (5B) Ratio of 4 aa deletion to inclusion isoform of PSEN1. Abundance (percent out of total PSEN1 transcripts) of PSEN1 (5C) exon 3b inclusion isoform, and (5D) intron 8 retention isoform, across cohorts. No isoform exhibited significantly different ratio or abundance across cohorts (P>0.05, One-Way ANOVA, for each). (5E) Distribution of various PSEN1 isoforms as a percentage of all full-length transcripts. All bars are mean and SD. Sporadic AD n=7, PSEN variant carrier n=7, control n=6.

FIGS. 6A-6F. Isoforms of PSEN2. (6A) Schematic showing the three most predominant isoforms identified for PSEN2, as well as the 77 bp intron splice site and the location of the N141I variant. (6B) Ratio of 1 aa deletion to inclusion isoform of PSEN2. Abundance (percent out of total PSEN2 transcripts) of PSEN2 (6C) exon 9B inclusion isoform, (6D) intron 77 inclusion isoform, and (6E) exon 6 skipping events, across cohorts. (6F) Distribution of various PSEN2 isoforms as a percentage of all full-length transcripts. P-values were determined by a One-Way ANOVA, followed by Tukey's multiple comparisons. * <0.05, ** <0.01, *** <0.001. All bars are mean and SD. Sporadic AD n=7, PSEN variant carrier n=7, control n=6.

FIG. 7. Alternative PSEN2 isoforms containing exon 9B. Shown is a schematic of PSEN2 splice isoforms containing exon 9B inclusion alone and in combination with additional intron retention and alternatively spliced exons in introns 8 and 9 that are shaded in blue. The length of the mRNA sequence (from exon 4 to exon 13) and the longest protein open reading frame are then listed. On the right is the average percentage of full-length transcripts from sporadic AD (SAD), control (Con) and PSEN variant carriers along with the combined percentage of reads that incorporate exon 9B at the bottom. wt=wildtype (canonical) PSEN2.

FIG. 8. Proportion of PSEN2 exon 9B reads as a function of age at death for each sample. Exon 9B inclusion was not correlated with age at death in any cohort (r=0.24, P (two-tailed)=0.30, Spearman correlation). Note: two control points overlap at age 88. Sporadic AD n=7, PSEN variant carrier n=7, control n=6.

FIGS. 9A, 9B. Alternative splicing profile at PSEN2 exon 6. (9A) Schematic of alternative splice junctions that are detected including the presence of the 77 bp intron retention sequence, the N141I variant position and an HMGA1A binding site. (9B) Proportion of reads mapping from the splice donor site at the end of exon 5 to one of three acceptor sites: a 77 bp intron retention product before exon 6, to exon 6, or to exon 7 (skipping exon 6).

FIGS. 10A, 10B. Allelic bias at PSEN1 and PSEN2 pathogenic variant positions. (10A) Percentage of PSEN2 isoforms with or without the 2 bp deletion and 77 bp intron retention. (10B) Percentage of transcripts with the wildtype allele or the PSEN variant (mutant allele) present. Sporadic AD n=7, PSEN variant carrier n=7, control n=6.

FIGS. 11A-11D. RNA splicing in Mayo Clinic RNA-seq samples. Proportion of spliced reads in cerebellum samples for PSEN2 (11A) exon 9B, and (11B) 77 bp intron retention product or exon 6 skipping. Proportion of spliced reads in temporal cortex samples for PSEN2 (11C) exon 9B, and (11D) 77 bp intron retention or exon 6 skipping products. PSP=progressive supranuclear palsy. P-values were determined by a One-Way ANOVA, followed by Tukey's multiple comparisons. * <0.05, ** <0.01, *** <0.001, **** <0.0001. All bars are mean and SD. Sporadic AD n=82, control n=76, pathological aging n=25, progressive supranuclear palsy (PSP) n=81 for cerebellum samples. Sporadic AD n=82, control n=74, pathological aging n=28, PSP n=80 for temporal cortex samples.

FIGS. 12A-12D. RNA editing in PSEN2 3′UTR. (12A) Schematic showing two 3′ UTRs identified for PSEN2, short (without Alu elements) and long (with Alu elements). (12B) Percentage of A-to-I editing of the AluJb and AluY sites, across cohorts. Sporadic AD n=7, PSEN variant carrier n=7, control n=6. (12C) Level of RNA editing in Alu elements in the PSEN2 3′UTR. P-values were determined by a One-Way ANOVA, followed by Tukey's multiple comparisons. * <0.05, **** <0.0001. All bars are mean and SD. Sporadic AD n=82, control n=76, pathological aging n=25, PSP n=81 (12D) Examples of individual reads with several edits (horizontal circles) and individual residues with multiple edits (vertical circles).

FIGS. 13A-13C. Additional RNA splicing quantification in Mayo Clinic RNA-seq samples. Proportion of spliced reads in cerebellum samples for PSEN2 (13A) 1 aa inclusion product, and for PSEN1 (13B) 4 aa inclusion product and (13C) exon 3B. No isoforms percentages were significantly different across cohorts (One-Way ANOVA). Sporadic AD n=82, control n=76, pathological aging n=25, PSP n=81.

FIG. 14. Age of death correlation with PSEN2 exon 9B levels in Mayo Clinic RNA-seq data from cerebellum. Percentage of PSEN2 exon 9B levels as a function of post-mortem interval (PMI) across cohorts. Sporadic AD n=82, control n=76, pathological aging n=25, PSP n=81.

FIGS. 15A-15C. Confirmation of PSEN2 exon 9B alternative splicing in parietal lobe and temporal gyrus brain regions in individuals with sporadic Alzheimer's disease (AD) versus control (Ctrl). (15A) RT-PCR products on an agarose gel with and without exon 9B. (15B) Levels of PSEN2 exon9B inclusion in temporal gyrus samples from control (n=15) versus AD (n=31); ***p<0.001. (15C) Levels in parietal lobe tissue from control (n=10) versus AD (n=22); *p<0.05.

FIG. 16. Amyloid beta (AB) 42:40 ratio from iPSC-derived neurons treated with PSEN2 exon 9B lentivirus at increasing multiplicity of infection (MOI).

FIGS. 17A, 17B. Tables of Gene Targets and Sequences. (17A) Genomic and (17B) cDNA.

FIG. 18. Sequences supporting the disclosure.

### DETAILED DESCRIPTION

Ranking as the most common form of both dementia and neurodegeneration, Alzheimer's disease is progressively debilitating and uniformly fatal. Almost six million Americans have the disease—a number that will continue to escalate as the population ages—and still there remains no effective treatment.

Originally identified through linkage analyses, autosomal dominant variants in PSEN1, PSEN2, and amyloid beta precursor protein (APP) lead to early-onset Alzheimer's disease (onset <65 years old) (Goate A, et al. Nature. Feb. 21, 1991; 349(6311):704-6.; Levy-Lahad et al. Science. Aug. 18, 1995; 269(5226):973-7.; Sherrington R, et al. Nature. Jun. 29, 1995; 375(6534):754-60.). These three risk genes are mechanistically united in the following way: either of the two presenilins form part of the γ-secretase complex, which cleaves APP to liberate a product called amyloid beta (Aβ) peptide. Though the mechanisms by which variants in PSEN1 and PSEN2 lead to Alzheimer's disease are still debated, it is commonly thought that changes in PSEN1 or PSEN2 bias APP cleavage to form versions of Aβ peptide that are more prone to aggregation. Aggregation of Aβ, in turn, is a hallmark pathology of AD.

PSEN1 and PSEN2 are highly similar (65% identical at the amino acid level). Despite their similarities, many more pathogenic variants have been documented in PSEN1 than in PSEN2 (20-70% versus 5%, respectively) (Bird T D. Alzheimer Disease Overview. In: Adam M P, et al, eds. GeneReviews(®). 1993.). This phenomenon may be explained by differences in localization (for example, PSEN2 plays a selective role in microglia (Fung S, et al. J Alzheimers Dis. 2020; 77(2):675-688.; Jayadev S, et al. J Neurochem. December 2013; 127(5):592-9.; Jayadev S, et al. PLoS One. Dec. 29 2010; 5(12):e15743.)) or function (PSEN1 has a greater effect on calcium signaling (Payne A J, et al. Receptors Clin Investig. 2015; 2(1):e449.)). It is also possible that pathogenic variants in PSEN2 are more prevalent than currently thought, but that cases with these variants are considered sporadic due to their later age of onset (Blauwendraat C, et al. Neurobiol Aging. January 2016; 37:208 e11-208 e17.).

The proportion of early-onset familial Alzheimer's disease cases attributed to variants in PSEN1 is 20%-70%, and those attributed to variants in PSEN2 is 5% (Bird T D. Alzheimer Disease Overview. In: Adam M P, et al, eds. GeneReviews(®). 1993.). In addition to playing a role in early-onset Alzheimer's disease, variants in both genes can also increase risk for, or cause, late-onset Alzheimer's disease (Cruchaga C, et al. PLoS One. 2012; 7(2):e31039.). It is generally thought that all disease-causing variants in these genes impair normal function of the γ-secretase complex resulting in abnormal cleavage of APP and aggregation of Aβ. Tellingly, of all identified Alzheimer's disease-related PSEN1 variants (there are now over 200) (Lanoiselee H M, et al. PLoS Med. March 2017; 14(3):e1002270.), not one leads solely to a truncation or absence of protein product (De Strooper B. EMBO Rep. February 2007; 8(2):141-6.). PSEN2 variants are not as common as those in PSEN1 (19 identified so far) (Lanoiselee H M, et al. PLoS Med. March 2017; 14(3):e1002270.), but they similarly do not appear to lead to haploinsufficiency. Based on these observations, it appears that PSEN1 and PSEN2 are obligated to form full-length transcripts. Determining whether this PSEN1 and PSEN2 are obligated to form full-length transcripts would notably increase the understanding of Alzheimer's disease pathogenesis.

Recently, the impact of an Alzheimer's disease-related PSEN2 variant, PSEN2 K115Efs (Lanoiselee H M, et al. PLoS Med. March 2017; 14(3):e1002270.), was characterized (Jayadev S, et al. Brain. April 2010; 133(Pt 4):1143-54.). This variant is a 2-base pair (bp) deletion, which should lead to a frameshift and premature stop codon, and therefore be the first known case of a loss-of-function variant in PSEN2. Instead, however, a novel intronic splice acceptor site was detected that added 77 bp to the transcript, which would restore the reading frame and generate a full-length PSEN2 product with 25 extra amino acids (Braggin J E, et al. Ann Clin Transl Neurol. April 2019; 6(4):762-777.). This transcript was specific to patient brain tissue and was not observed in fibroblasts. It was also observed that the same intron retention splicing pattern in brain tissues from patients with sporadic Alzheimer's disease (i.e., lacking the 2 bp deletion) and observed use of this alternative splice site in RNA sequencing data of anterior frontal cortex in the Mount Sinai Brain Bank (MSBB), indicating that alternative splicing of familial Alzheimer's disease risk genes may also play a role in sporadic Alzheimer's disease.

The reading frame of PSEN2 K115Efs could also be restored by the omission of exon 6. Alternative splicing leading to skipping of PSEN2 exon 6 occurs under hypoxic conditions—a condition increasingly implicated in Alzheimer's disease (Iadecola C. Nat Rev Neurosci. May 2004; 5(5):347-60.)—and the truncated splicing product is elevated in the brains of patients with sporadic Alzheimer's disease (Sato N, et al. J Neurochem. June 1999; 72(6):2498-505.). This splicing event is thought to decrease the unfolded protein response and increase γ-secretase activity (Moussavi Nik S H, et al. Hum Mol Genet. Jul. 1, 2015; 24(13):3662-78.). Splicing is altered by high mobility group A protein 1a (HMGA1a), which binds exon 6 and causes it to be skipped, resulting in a premature stop codon and thus a truncated protein product (Moussavi Nik S H, et al. Exp Cell Res. Jul. 1, 2011; 317(11):1503-12.; Manabe T, et al. Cell Death Differ. June 2003; 10(6):698-708.). PSEN2 K115Efs acts in a manner similar to PSEN2 transcripts with exon 6 spliced out—that is, both form similar truncated products—but together, they form an almost full-length PSEN2 product, lacking exon 6.

Various embodiments of the present disclosure relate to treating and diagnosing tauopathies utilizing transcript variants of PSEN2. Reduction of transcript variants of PSEN2 and/or preservation of canonical splicing can be beneficial in the treatment of tauopathies such as Alzheimer's disease.

In particular embodiments, a method of treating or inhibiting tauopathies includes the administration of an inhibitory nucleic acid molecule that targets a pathogenic transcript variant of PSEN2 thereby reducing the expression of the pathogenic transcript variant in a subject in need thereof. In particular embodiments, the tauopathy includes sporadic Alzheimer's disease. In particular embodiments, the tauopathy includes familial Alzheimer's disease or progressive supranuclear palsy.

In particular embodiments, the inhibitory nucleic acid molecule that targets a pathogenic transcript variant of PSEN2 includes antisense oligonucleotides, short hairpin RNA (shRNA), small interfering RNA (siRNA), and/or microRNA (miRNA) that hybridize to a portion of the pathogenic transcript variant of PSEN2.

In particular embodiments, a method of treating or inhibiting tauopathies includes the administration of splice-switching oligonucleotides that target a pathogenic transcript variant of PSEN2. In particular embodiments, the splice-switching oligonucleotides hybridize to the pathogenic transcript variant of PSEN2 and shift splicing patterns thereby restoring the expression of wildtype PSEN2.

In particular embodiments, the pathogenic transcript variants of PSEN2 include a transcript with the inclusion of exon 9B. In particular embodiments, exon 9B includes the sequence as set forth in SEQ ID NO: 2. In particular embodiments, the transcript with the inclusion of exon 9B includes the sequence as set forth in SEQ ID NO: 3, SEQ ID NO: 4, or SEQ ID NO: 5. In particular embodiments, the pathogenic transcript variant of PSEN2 include a transcript with the inclusion of 77 bp of the intronic sequence prior to exon 6. In particular embodiments, the 77 bp intronic sequence includes the sequence as set forth in SEQ ID NO: 6. In particular embodiments, the transcript with the inclusion of the 77 bp intronic sequence includes the sequence as set forth in SEQ ID NO: 7. In particular embodiments, a pathogenic transcript variant of PSEN2 is not wildtype PSEN2. In particular embodiments, wildtype PSEN2 includes the sequence as set forth in SEQ ID NO: 1.

Methods of diagnosing tauopathies are also provided. In particular embodiments, a tauopathy includes sporadic Alzheimer's disease. In particular embodiments, a subject having a tauopathy has more transcript variants of PSEN2 compared to subjects without a neuropathologic disease. In particular embodiments, a method of diagnosing a subject includes detecting a level of one of more transcript variants of PSEN2 in a tissue sample of the subject. In particular embodiments, the level of transcript variants of PSEN2 will be higher in subjects with the tauopathy compared to a reference level. In particular embodiments, a method of diagnosing a subject with a tauopathy includes quantifying select alternative splicing isoforms of PSEN2 reads compared to total PSEN2 reads within a tissue sample of the subject. In particular embodiments, the proportion of select alternative splicing isoforms of PSEN2 will be higher in subjects with the tauopathy compared to a reference level. In particular embodiments, the reference level is the level of transcript variants of PSEN2 or the proportion of select alternative splicing isoforms of PSEN2 from tissue samples of subjects without a neuropathology.

Embodiments of the present disclosure will now be described with reference to the accompanying figures.

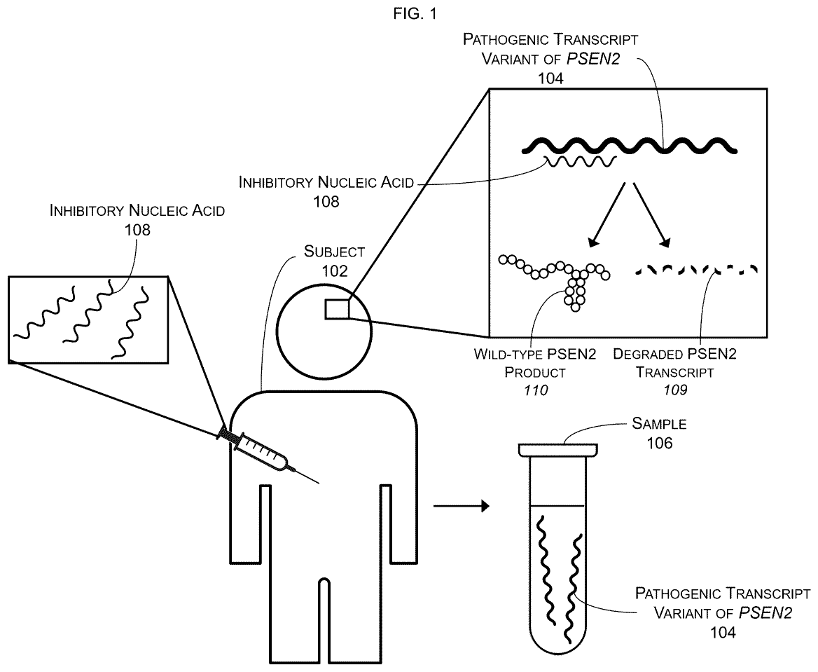

FIG. 1 describes an example environment 100 for administration of inhibitory nucleic acids that specifically bind pathogenic transcript variants of PSEN2. In this example, a subject 102 suffers from a pathology associated with the presence of transcript variants of PSEN2. The subject 102, for example, is a human or other animal. The subject 102, in various examples, has a tauopathy. In some examples, the tauopathy is Alzheimer's disease. In some examples, the tauopathy is sporadic Alzheimer's disease. In some examples, the tauopathy is familial Alzheimer's disease. In some instances, transcript variants of PSEN2 104 are present in the subject 102. For instance, transcript variants of PSEN2 104 may be present in the central nervous system of the subject 102. In some examples, transcript variants of PSEN2 104 are present in the brain of the subject 102. In some examples, transcript variants of PSEN2 104 are present in cerebrospinal fluid (CSF) of the subject 102. In some examples, transcript variants of PSEN2 104 are present in microglia of the subject 102. In some examples, transcript variants of PSEN2 104 are present in the blood of the subject 102.

In various examples, at least one care provider may diagnose the subject 102 with a tauopathy. For example, the care provider(s) (e.g., a clinician) may perform a spinal tap to collect a sample 106 from the subject 102. In some examples, the sample 106 includes cerebrospinal fluid (CSF) of the subject 102. The care provider(s) (e.g., a medical technologist) may perform analysis on the sample 106 to determine a level of amyloid beta (Aβ) accumulation, amyloid plaques, or tau protein tangles in the subject 102. In some examples, the care provider(s) (e.g., a radiologist) may identify Aβ accumulation, amyloid plaques, or tau protein tangles in the brain of the subject 102 using medical imaging techniques, including positron emission tomography (PET) or magnetic resonance imaging (MRI) scans. In some examples, the care provider(s) may determine the presence of genetic markers associated with tauopathies in the subject 102. In some instances, the care provider(s) (e.g., a clinician) may perform cognitive and/or neurological assessments on the subject 102.

In various examples, early diagnosis of a tauopathy may be beneficial. Early diagnosis may enable participation in clinical trials, improved quality of life, and comprehensive treatment and support. For example, Alzheimer's disease is generally diagnosed by detecting Aβ accumulation, amyloid plaques, or tau protein tangles. However, transcript (i.e., RNA) biomarkers have been found to have higher sensitivity and specificity than protein biomarkers (Xi, et al. Noncoding RNA. March 2017; 3(1):9).

In some examples, the care provider(s) (e.g., a clinician) may diagnose the subject 102 with a tauopathy associated with the presence of transcript variants of PSEN2 104. According to various embodiments of the present disclosure, alternative splicing patterns of PSEN2, which lead to the generation of alternative splicing isoforms (i.e., transcript variants) are associated with a tauopathy. In particular embodiments, detection of the transcript variants of PSEN2 104 is useful in diagnosis and/or prognosis of a condition of the subject 102. The term “diagnosis”, as used herein, refers to evaluation of the presence or properties of pathological states or lack thereof. As used herein, prognosis of the subject's condition can refer to evaluating indicia of a tauopathy at a given time point and comparing it to the same indicia of the tauopathy taken at an earlier time point, wherein the comparison is indicative of a progression of the tauopathy in the subject 102.

In some examples, the care provider(s) may determine that the transcript variants of PSEN2 104 are present in the subject 102. The care provider(s) may analyze the sample 106 to detect transcript variants of PSEN2 104. In particular embodiments, the sample 106 is a blood sample or a CSF sample. Transcript variants of PSEN2 104 may be detected by Reverse-Transcription (RT) Polymerase Chain Reaction (PCR), quantitative PCR (qPCR), RNA sequencing (RNA-Seq), isoform sequencing (Iso-Seq), Cap Analysis of Gene Expression (CAGE), Polyadenylation Site Sequencing (PAS-Seq), Long-Read Sequencing (e.g., PacBio sequencing, Nanopore sequencing), RNase Protection Assay, northern blotting, microarray analysis, or another technique known in the art. In particular embodiments, a cDNA sequence associated with the transcript variant of PSEN2 104 may be detected.

In particular cases, the transcript variant of PSEN2 104 includes exon 9B. In particular embodiments, exon 9B includes a sequence having at least 70% sequence identity to SEQ ID NO: 2. In particular embodiments, exon 9B includes a sequence having at least 80%, 90%, 95%, 97%, or 99% sequence identity to SEQ ID NO: 2. In particular embodiments, exon 9B includes the sequence as set forth in SEQ ID NO: 2.

In particular embodiments, the transcript variant of PSEN2 104 with the inclusion of exon 9B includes a sequence having at least 70% sequence identity to SEQ ID NO: 3, SEQ ID NO: 4, or SEQ ID NO: 5. In particular embodiments, the transcript with the inclusion of exon 9B includes a sequence having at least 80%, 90%, 95%, 97%, or 99% sequence identity to SEQ ID NO: 3, SEQ ID NO: 4, or SEQ ID NO: 5. In particular embodiments, the transcript with the inclusion of exon 9B includes the sequence as set forth in SEQ ID NO: 3, SEQ ID NO: 4, or SEQ ID NO: 5.

In particular embodiments, the transcript variant of PSEN2 104 include a transcript with the inclusion of 77 bp of the intronic sequence prior to exon 6. In particular embodiments, the 77 bp intronic sequence includes a sequence having at least 70% sequence identity to SEQ ID NO: 6. In particular embodiments, the 77 bp intronic sequence includes a sequence having at least 80%, 90%, 95%, 97%, or 99% sequence identity to SEQ ID NO: 6. In particular embodiments, the 77 bp intronic sequence includes the sequence as set forth in SEQ ID NO: 6.

In particular embodiments, the transcript variant of PSEN2 104 with the inclusion of the 77 bp intronic sequence includes a sequence having at least 70% sequence identity to SEQ ID NO: 7. In particular embodiments, the transcript with the inclusion of the 77 bp intronic sequence includes a sequence having at least 80%, 90%, 95%, 97%, or 99% sequence identity to SEQ ID NO: 7. In particular embodiments, the transcript with the inclusion of the 77 bp intronic sequence includes the sequence as set forth in SEQ ID NO: 7.

In particular embodiments, a transcript variant of PSEN2 104 is not wildtype PSEN2. In particular embodiments, wildtype PSEN2 includes a sequence having at least 70% sequence identity to SEQ ID NO: 1. In particular embodiments, wildtype PSEN2 includes a sequence having at least 80%, 90%, 95%, 97%, or 99% sequence identity to SEQ ID NO: 1. In particular embodiments, wildtype PSEN2 includes the sequence as set forth in SEQ ID NO: 1.

In some examples, the care provider(s) may diagnose the subject 102 based on a ratio of a level of the transcript variants of PSEN2 104 to a level of wild-type transcripts of PSEN2. In particular embodiments, the care provider(s) may compare the level of the transcript variants of PSEN2 104 in the subject 102 to a reference level. In particular embodiments, the care provider(s) may compare a ratio of pathogenic transcript variants 104 to wild-type transcripts to a reference ratio of pathogenic transcript variants 104 to wild-type transcripts. Reference levels may be obtained from a population of subjects without a neurodegenerative disease (e.g., Alzheimer's disease). As is understood by one of ordinary skill in the art, the reference level can be based on any mathematical or statistical formula useful and known in the art for arriving at a meaningful aggregate reference level from a collection of individual data points; e.g., mean, median, median of the mean, etc. In particular embodiments, a reference level may be obtained from a population of subjects including those that have a neurodegenerative disease (e.g., Alzheimer's disease).

In particular embodiments, conclusions are drawn based on whether a sample value is statistically significantly different or not statistically significantly different from a reference level. A measure is not statistically significantly different if the difference is within a level that would be expected to occur based on chance alone. In contrast, a statistically significant difference or increase is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular data point, where the data point is the result of random chance alone. A result is often considered significant (not random chance) at a p-value less than or equal to 0.05. In particular embodiments, a sample value is “comparable to” a reference level derived from a normal control population if the sample value and the reference level are not statistically significantly different.

Currently, treatments options are limited for tauopathies, such as Alzheimer's disease. In various examples, it may be beneficial to inhibit the expression of a pathogenic transcript variant associated with the disease. Blocking the expression of a pathogenic transcript variant (e.g., the transcript variant of PSEN2 104), in various embodiments, may slow or stop the progression of the tauopathy by reducing or eliminating the protein product of the pathogenic transcript variant. In some examples, the pathogenic transcript variant is the pathogenic transcript variant of PSEN2 104.

In various embodiments of the present disclosure, the subject 102 is administered an inhibitory nucleic acid 108 to treat or inhibit the progression of the tauopathy. In particular embodiments, the inhibitory nucleic acid 108 inhibits the expression of transcript variant of PSEN2 104. In some examples, the inhibitory nucleic acid 108 specifically binds one or more disclosed transcript variants of PSEN2 104.

In various embodiments, the inhibitory nucleic acid 108 is an antisense oligonucleotide, short hairpin RNA (shRNA), small interfering RNA (siRNA), and/or microRNA (miRNA) that hybridizes to a portion of the transcript variant of PSEN2 104. The inhibitory nucleic acid 108 provides a degraded PSEN2 transcript 109, and translation of the transcript variant of PSEN2 104 is inhibited. MiRNA is usually 18-27 nucleotides (nt) in length and is capable of either directly degrading its messenger RNA (mRNA) target or suppressing the protein translation of its targeted mRNA, depending on the complementarity between the miRNA and its target. SiRNA is double-stranded RNA that primarily triggers mRNA degradation. ShRNA is single-stranded RNA including siRNA or mature double-stranded miRNA ligated by an adequate short spacer. ShRNA is generally cleaved into siRNA or miRNA and can thus be used as a “knock-down” tool to silence or down-regulate gene expression through RNA inactivation.

In particular embodiments, the inhibitory nucleic acid 108 is a splice-switching oligonucleotide that targets the transcript variant of PSEN2 104. In particular embodiments, the splice-switching oligonucleotides hybridize to the transcript variant of PSEN2 104 and shift splicing patterns. Based on shifting splicing patterns, the expression of wildtype PSEN2 product 110 may be restored. In some embodiments, a method of treating or inhibiting tauopathies includes the administration of splice-switching oligonucleotides that target the pathogenic transcript variant of PSEN2 104 and at least one of antisense oligonucleotides, shRNA, siRNA, or miRNA that inhibit the expression of transcript variant of PSEN2 104.

In particular embodiments, the inhibitory nucleic acid 108 has sufficient sequence complementarity to a portion of the transcript variant of PSEN2 104. In various examples, a nucleic acid with sufficient sequence complementarity to a second nucleic acid can hybridize to the second nucleic acid and reduce translation of the second nucleic acid. In particular embodiments, “sufficient complementarity” refers to at least 70%, at least 75%, at least 80%, at least 85%, at least 90%, at least 95%, at least 96%, at least 97%, at least 98%, or at least 99% sequence complementarity. In particular embodiments, a nucleic acid binding a sequence to which the nucleic acid has sufficient complementarity results in at least 30%, at least 40%, at least 50%, at least 60%, at least 70%, at least 75%, at least 80%, at least 85%, at least 90%, at least 95%, at least 96%, at least 97%, at least 98%, or at least 99% reduction in translation of the sequence.

In particular embodiments, a complementary nucleic acid reduces translation of the transcript variant of PSEN2 104. The sequence of the inhibitory nucleic acid 108 need not be 100% complementary to that of its target nucleic acid (e.g., the transcript variant of PSEN2 104) to hybridize. In certain embodiments, the inhibitory nucleic acid 108 can include at least 70%, at least 75%, at least 80%, at least 85%, at least 90%, at least 95%, at least 96%, at least 97%, at least 98%, or at least 99% sequence complementarity to a target region within the target nucleic acid sequence (e.g., the transcript variant of PSEN2 104) to which they are targeted. In certain embodiments, administration of the inhibitory nucleic acid 108 is associated with at least 30%, at least 40%, at least 50%, at least 60%, at least 70%, at least 75%, at least 80%, at least 85%, at least 90%, at least 95%, at least 96%, at least 97%, at least 98%, or at least 99% reduction in the translation of the transcript variant of PSEN2 104.

The inhibitory nucleic acid 108 need not hybridize to all nucleotides in a target sequence (e.g., the transcript variant of PSEN2 104), and the nucleotides to which it does hybridize may be contiguous or noncontiguous. Inhibitory nucleic acids (e.g., the inhibitory nucleic acid 108) may hybridize over one or more segments of a sequence, such that intervening or adjacent segments are not involved in the hybridization event (e.g., a loop structure or hairpin structure may be formed). In certain embodiments, the inhibitory nucleic acid 108 hybridizes to noncontiguous nucleotides.

In various implementations of the present disclosure, inhibitory nucleic acids also include nucleic acid molecule that hybridize under stringent hybridization conditions to a sequence disclosed herein (e.g., a pathogenic transcript variant of PSEN2) and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

In particular embodiments, the inhibitory nucleic acid 108 is conjugated to a tag. Examples of tags include fluorescent probes (e.g., fluoresceine, rhodamine, cyanine dyes, SYBR green, etc.), affinity tags (biotin, poly(A) tail tags, poly(T) tail tags, His-tags, FLAG tags, Myc tags, hemagglutinin (HA) tags, AviTag, SNAP tags, etc), barcodes, adapters, hybridization probes (e.g., TaqMan probes, Scorpion probes, FRET probes, etc.), molecular beacons (e.g., locked nucleic acid (LNA) probes, peptide nucleic acid (PNA) probes, etc.), chromogenic probes (colorimetric probes, gold nanoparticles, etc.), or other detectable labels.

Although FIG. 1 illustrates the administration of the inhibitory nucleic acid 108 to a human subject 102, embodiments of the present disclosure are not so limited. Methods disclosed herein include treating humans, non-human primates, and other animals, including research animals (monkeys, rats, mice, fish, etc). Treating subjects includes delivering therapeutically effective amounts. Therapeutically effective amounts include those that provide effective amounts, prophylactic treatments and/or therapeutic treatments.

An “effective amount” is the amount of a formulation necessary to result in a desired physiological change in the subject. For example, an effective amount can provide a change in a metric associated with a tauopathy (e.g., a level of Aβ accumulation, a level of amyloid plaques, a level of tau protein tangles, physical symptoms, cognitive symptoms, reduced or neurological symptoms, an Aβ42:40 ratio, a level of pathogenic transcript variants of PSEN2 with exon 9B, and/or a level of pathogenic transcript variants of PSEN2 with the inclusion of 77 bp of the intronic sequence prior to exon 6). Effective amounts are often administered for research purposes. Effective amounts disclosed herein can cause a statistically significant effect in an animal model or in vitro assay relevant to the assessment of a tauopathy's development or progression.

A “prophylactic treatment” includes a treatment administered to a subject (e.g., the subject 102) who does not display signs or symptoms of a tauopathy or displays only early signs or symptoms of a tauopathy such that treatment is administered for the purpose of diminishing or decreasing the risk of developing the tauopathy further. Thus, a prophylactic treatment functions as a preventative treatment against a pathogenic PSEN2 transcript variant-expressing tauopathy. In particular embodiments, prophylactic treatments reduce or delay physical, cognitive, or neurological symptoms associated with a tauopathy from occurring.

A “therapeutic treatment” includes a treatment administered to a subject (e.g., the subject 102) who displays symptoms or signs of a tauopathy and is administered to the subject for the purpose of diminishing or eliminating those signs or symptoms of the tauopathy. The therapeutic treatment can reduce, control, or eliminate the presence or activity of the tauopathy and/or reduce control or eliminate side effects of the tauopathy.

Function as an effective amount, prophylactic treatment or therapeutic treatment are not mutually exclusive, and in particular embodiments, administered dosages may accomplish more than one treatment type.

In particular embodiments, therapeutically effective amounts induce a reduced progression of a tauopathy. In particular embodiments, the reduced progression includes reduced or stabilized level of Aβ accumulation, reduced or stabilized level of amyloid plaques, reduced or stabilized level of tau protein tangles, reduced or stabilized physical symptoms, reduced or stabilized cognitive symptoms, reduced or stabilized neurological symptoms, increased or stabilized Aβ42:40 ratio, reduced or stabilized level of pathogenic transcript variants of PSEN2 with exon 9B, and/or reduced or stabilized level of pathogenic transcript variants of PSEN2 with the inclusion of 77 bp of the intronic sequence prior to exon 6.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of cancer, stage of, previous or concurrent therapeutic interventions, idiopathy of the subject and route of administration.

Therapeutically effective amounts of formulations can include 10⁴ to 10⁹ cells/kg body weight, or 10³ to 10¹¹ cells/kg body weight. Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

Therapeutically effective amounts of compositions can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular embodiments, the treatment protocol may be dictated by a clinical trial protocol or an FDA-approved treatment protocol.

Therapeutically effective amounts can be administered by, e.g., injection, infusion, perfusion, or lavage. Routes of administration can include bolus intravenous, intradermal, intraarterial, intraparenteral, intranodal, intralymphatic, intraperitoneal, intralesional, intraprostatic, intravaginal, intrarectal, topical, intrathecal, intratumoral, intramuscular, intravesical, and/or subcutaneous administration. In particular embodiments, cell formulations and/or modifying formulations are administered by intravesically.

Reagents (e.g., inhibitory nucleic acids) can be provided as part of a composition formulated for administration to subjects. The formulated reagent can include a recombinant protein and/or a nucleic acid, either or both optionally provided within a nanoparticle. In certain examples, proteins and/or nucleic acids can be encoded by an administered vector (e.g., viral vector such as lentiviral vectors). Regardless of particular form, reagents can be referred to as active ingredient(s).

In particular embodiments, active ingredients are provided as part of a composition that can include, for example, at least 0.1% w/v or w/w of active ingredient(s), at least 1% w/v or w/w of active ingredient(s), at least 10% w/v or w/w of active ingredient(s), at least 20% w/v or w/w of active ingredient(s), at least 30% w/v or w/w of active ingredient(s), at least 40% w/v or w/w of active ingredient(s), at least 50% w/v or w/w of active ingredient(s), at least 60% w/v or w/w of active ingredient(s), at least 70% w/v or w/w of active ingredient(s), at least 80% w/v or w/w of active ingredient(s), at least 90% w/v or w/w of active ingredient(s), at least 95% w/v or w/w of active ingredient(s), or at least 99% w/v or w/w of active ingredient(s).

The compositions disclosed herein can be formulated for administration by, for example, injection, inhalation, infusion, perfusion, lavage or ingestion. The compositions can further be formulated for, for example, intravenous, intradermal, intraarterial, intranodal, intralymphatic, intraperitoneal, intralesional, intraprostatic, intravaginal, intrarectal, topical, intrathecal, intratumoral, intramuscular, intravesicular, oral and/or subcutaneous administration and more particularly by intravenous, intradermal, intraarterial, intranodal, intralymphatic, intraperitoneal, intralesional, intraprostatic, intravaginal, intrarectal, topical, intrathecal, intratumoral, intramuscular, intravesicular, oral and/or subcutaneous injection.

For injection, compositions can be formulated as aqueous solutions, such as in buffers including Hanks' solution, Ringer's solution, or physiological saline. The aqueous solutions can contain formulatory agents such as suspending, stabilizing and/or dispersing agents. Alternatively, the formulation can be in lyophilized and/or powder form for constitution with a suitable vehicle, e.g., sterile pyrogen-free water, before use.

Any composition formulation disclosed herein can advantageously include any other pharmaceutically acceptable carriers which include those that do not produce significantly adverse, allergic or other untoward reactions that outweigh the benefit of administration, whether for research or immunization efforts. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety and purity standards as required by United States FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants.

Exemplary buffering agents include citrate buffers, succinate buffers, tartrate buffers, fumarate buffers, gluconate buffers, oxalate buffers, lactate buffers, acetate buffers, phosphate buffers, histidine buffers and/or trimethylamine salts.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol and 3-pentanol.

Exemplary isotonic agents include polyhydric sugar alcohols including trihydric or higher sugar alcohols, such as glycerin, erythritol, arabitol, xylitol, sorbitol or mannitol.

Exemplary stabilizers include organic sugars, polyhydric sugar alcohols, polyethylene glycol, sulfur-containing reducing agents, amino acids, low molecular weight polypeptides, proteins, immunoglobulins, hydrophilic polymers or polysaccharides.

As indicated, in particular embodiments, a reagent may be encapsulated in or conjugated to nanoparticles for administration. Nanoparticles can include lipid nanoparticles, a polymeric nanoparticle, surfactant-based emulsions, micelles, silica nanoparticles, albumin nanoparticles, nanotubes (e.g., carbon nanotubes), dendrimers, microparticles, and/or nanoparticles that are developed using a combination of nanomaterials such as lipid-polymer nanoparticles.

The Exemplary Embodiments and Experimental Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Exemplary Embodiments

1. A method of treating a neurodegenerative disease including
   administering a therapeutically effective amount of a composition to a subject in need thereof, wherein the composition includes a reagent that reduces expression of a pathogenic transcript variant of PSEN2 in the subject, thereby treating the subject in need thereof.
2. The method of embodiment 1, wherein the neurodegenerative disease is a tauopathy.
2. The method of embodiment 2, wherein the tauopathy includes Alzheimer's disease.
3. The method of embodiment 3, wherein Alzheimer's disease includes sporadic Alzheimer's disease.
4. The method of embodiment 3, wherein Alzheimer's disease includes familial Alzheimer's disease.
5. The method of embodiment 2, wherein the tauopathy includes progressive supranuclear palsy.
6. The method of any of embodiments 1-6, wherein the reagent includes a nucleic acid having sufficient sequence complementarity to the pathogenic transcript variant of PSEN2.
7. The method of embodiment 7, wherein the pathogenic transcript variant of PSEN2 includes a transcript with the inclusion of exon 9B.
8. The method of embodiment 8, wherein the reagent hybridizes to a pathogenic transcript variant of PSEN2 including the sequence set forth in SEQ ID NO: 3 or a sequence with at least 90% identity to the sequence set forth in SEQ ID NO: 3.
9. The method of embodiment 8 or 9, wherein the reagent has at least 95% reverse complementarity to the sequence set forth in SEQ ID NO: 3.
10. The method of any of embodiments 8-10, wherein the reagent is the reverse complement of the sequence set forth in SEQ ID NO: 3.
11. The method of any of embodiments 8-11, wherein the reagent hybridizes to a pathogenic transcript variant of PSEN2 including the sequence set forth in SEQ ID NO: 4 or a sequence with at least 90% identity to the sequence set forth in SEQ ID NO: 4.
12. The method of any of embodiments 8-12, wherein the reagent has at least 95% reverse complementarity to the sequence set forth in SEQ ID NO: 4.
13. The method of any of embodiments 8-13, wherein the reagent is the reverse complement of the sequence set forth in SEQ ID NO: 4.
14. The method of any of embodiments 8-14, wherein the reagent hybridizes to a pathogenic transcript variant of PSEN2 including the sequence set forth in SEQ ID NO: 5 or a sequence with at least 90% identity to the sequence set forth in SEQ ID NO: 5.
15. The method of any of embodiments 8-15, wherein the reagent has at least 95% reverse complementarity to the sequence set forth in SEQ ID NO: 5.
16. The method of any of embodiments 8-16, wherein the reagent is the reverse complement of the sequence set forth in SEQ ID NO: 5.
17. The method of any of embodiments 7-17, wherein the pathogenic transcript variant of PSEN2 includes a transcript with the inclusion of 77 bp of the intronic sequence prior to exon 6.
18. The method of embodiment 18, wherein the reagent hybridizes to a pathogenic transcript variant of PSEN2 including the sequence set forth in SEQ ID NO: 7 or a sequence with at least 90% identity to the sequence set forth in SEQ ID NO: 7.
19. The method of embodiment 18 or 19, wherein the reagent has at least 95% reverse complementarity to the sequence set forth in SEQ ID NO: 7.
20. The method of any of embodiments 18-20, wherein the reagent is the reverse complement of the sequence set forth in SEQ ID NO: 7.
21. The method of any of embodiments 7-21, wherein the pathogenic transcript variant of PSEN2 includes the sequence set forth in SEQ ID NO: 2, SEQ ID NO: 3, SEQ ID NO: 4, SEQ ID NO: 5, SEQ ID NO: 6, or SEQ ID NO: 7 or a sequence having 90% sequence identity to the sequence set forth in SEQ ID NO: 2, SEQ ID NO: 3, SEQ ID NO: 4, SEQ ID NO: 5, SEQ ID NO: 6, or SEQ ID NO: 7.
22. The method of any of embodiments 7-22, wherein the pathogenic transcript variant of PSEN2 is not wildtype PSEN2 transcript.
23. The method of embodiment 23, wherein the wildtype PSEN2 transcript is set forth in SEQ ID NO: 1.
24. The method of any of embodiments 7-24, wherein the reagent includes an inhibitory nucleic acid molecule.
25. The method of embodiment 25, wherein the inhibitory nucleic acid molecule includes antisense oligonucleotides, short hairpin RNA (shRNA), small interfering RNA (siRNA), and/or microRNA (miRNA).
26. The method of embodiment 25 or 26, wherein the inhibitory nucleic acid molecule hybridizes to a portion of a sequence set forth in SEQ ID NO: 2 or a sequence having 90% sequence identity to the sequence set forth in SEQ ID NO: 2.
27. The method of any of embodiments 25-27, wherein the inhibitory nucleic acid molecule has at least 95% reverse complementarity to the sequence set forth in SEQ ID NO: 2.
28. The method of any of embodiments 25-28, wherein the inhibitory nucleic acid molecule is the reverse complement of the sequence set forth in SEQ ID NO: 2.
29. The method of any of embodiments 25-29, wherein the inhibitory nucleic acid molecule hybridizes to a portion of a sequence set forth in SEQ ID NO: 6 or a sequence having 90% sequence identity to the sequence set forth in SEQ ID NO: 6.
30. The method of any of embodiments 25-30, wherein the inhibitory nucleic acid molecule has at least 95% reverse complementarity to the sequence set forth in SEQ ID NO: 6.
31. The method of any of embodiments 25-31, wherein the inhibitory nucleic acid molecule is the reverse complement of the sequence set forth in SEQ ID NO: 6.
32. The method of any of embodiments 25-32, wherein the inhibitory nucleic acid molecule hybridizes to a portion of a sequence set forth in SEQ ID NO: 2, SEQ ID NO: 3, SEQ ID NO: 4, SEQ ID NO: 5, SEQ ID NO: 6, or SEQ ID NO: 7 or a sequence having 90% sequence identity to the sequence set forth in SEQ ID NO: 2, SEQ ID NO: 3, SEQ ID NO: 4, SEQ ID NO: 5, SEQ ID NO: 6, or SEQ ID NO: 7.
33. The method of any of embodiments 7-33, wherein the reagent includes splice-switching oligonucleotides.
34. The method of embodiment 34, wherein the splice-switching oligonucleotides hybridize to a portion of a sequence set forth in SEQ ID NO: 2 or a sequence having 90% sequence identity to the sequence set forth in SEQ ID NO: 2 thereby shifting splicing patterns to restore expression of wildtype PSEN2.
35. The method of embodiment 34 or 35, wherein the splice-switching oligonucleotides have at least 95% reverse complementarity to the sequence set forth in SEQ ID NO: 2.
36. The method of any of embodiments 34-36, wherein the splice-switching oligonucleotides are the reverse complement of the sequence set forth in SEQ ID NO: 2.
37. The method of any of embodiments 34-37, wherein the splice-switching oligonucleotides hybridize to a portion of a sequence set forth in SEQ ID NO: 6 or a sequence having 90% sequence identity to the sequence set forth in SEQ ID NO: 6 thereby shifting splicing patterns to restore expression of wildtype PSEN2.
38. The method of any of embodiments 34-38, wherein the splice-switching oligonucleotides have at least 95% reverse complementarity to the sequence set forth in SEQ ID NO: 6.
39. The method of any of embodiments 34-39, wherein the splice-switching oligonucleotides are the reverse complement of the sequence set forth in SEQ ID NO: 6.
40. The method of any of embodiments 34-40, wherein splice-switching oligonucleotides hybridize to a portion of a sequence set forth in SEQ ID NO: 2, SEQ ID NO: 3, SEQ ID NO: 4, SEQ ID NO: 5, SEQ ID NO: 6, or SEQ ID NO: 7 or a sequence having 90% sequence identity to the sequence set forth in SEQ ID NO: 2, SEQ ID NO: 3, SEQ ID NO: 4, SEQ ID NO: 5, SEQ ID NO: 6, or SEQ ID NO: 7.
41. The method of any of embodiments 1-41, wherein the reagent includes a vector encoding a nucleic acid having sufficient sequence complementarity to the pathogenic transcript variant of PSEN2.
42. The method of embodiment 42, wherein the vector is a viral vector.
43. A composition including a reagent that targets a pathogenic transcript variant of PSEN2, thereby inhibiting gene expression of the pathogenic transcript variant of PSEN2.
44. The composition of embodiment 44, wherein the reagent has sufficient complementarity to the pathogenic transcript variant of PSEN2.
45. The composition of embodiment 45, wherein the reagent includes an inhibitory nucleic acid molecule.
46. The composition of embodiment 46, wherein the inhibitory nucleic acid molecule includes antisense oligonucleotides, short hairpin RNA (shRNA), small interfering RNA (siRNA), and/or microRNA (miRNA).
47. The composition of embodiment 46 or 47, wherein the inhibitory nucleic acid molecule hybridizes to a portion of a sequence set forth in SEQ ID NO: 2 or a sequence having 90% sequence identity to the sequence set forth in SEQ ID NO: 2.
48. The composition of any of embodiments 46-48, wherein the inhibitory nucleic acid molecule has at least 95% reverse complementarity to the sequence set forth in SEQ ID NO: 2.
49. The composition of any of embodiments 46-49, wherein the inhibitory nucleic acid molecule is the reverse complement of the sequence set forth in SEQ ID NO: 2.
50. The composition of any of embodiments 46-50, wherein the inhibitory nucleic acid molecule hybridizes to a portion of a sequence set forth in SEQ ID NO: 6 or a sequence having 90% sequence identity to the sequence set forth in SEQ ID NO: 6.
51. The composition of any of embodiments 46-51, wherein the inhibitory nucleic acid molecule has at least 95% reverse complementarity to the sequence set forth in SEQ ID NO: 6.
52. The composition of any of embodiments 46-52, wherein the inhibitory nucleic acid molecule is the reverse complement of the sequence set forth in SEQ ID NO: 6.
53. The composition of any of embodiments 46-53, wherein the reagent includes a vector encoding a nucleic acid having sufficient sequence complementarity to the pathogenic transcript variant of PSEN2.
54. The composition of embodiment 54, wherein the vector is a viral vector
55. A polynucleotide complementary to a pathogenic transcript variant of PSEN2 conjugated to a tag.
56. The polynucleotide of embodiment 56, wherein the polynucleotide includes the sequence set forth in SEQ ID NO: SEQ ID NO: 2, SEQ ID NO: 3, SEQ ID NO: 4, SEQ ID NO: 5, SEQ ID NO: 6, or SEQ ID NO: 7 or a sequence having 90% sequence identity to the sequence set forth in SEQ ID NO: 2, SEQ ID NO: 3, SEQ ID NO: 4, SEQ ID NO: 5, SEQ ID NO: 6, or SEQ ID NO: 7.
57. The polynucleotide of embodiment 56 or 57, wherein the tag includes a fluorescent probe, an affinity tag, a barcode, an adapter, a hybridization probe, a molecular beacon, or a chromogenic probe.
58. A kit for detecting a pathogenic transcript variant of PSEN2 including reagents for reverse transcription of the pathogenic transcript variant of PSEN2 into a cDNA, reagents for amplification of the cDNA, and reagents for detection of the cDNA.
59. A method of diagnosing a tauopathy including:
   obtaining a tissue sample derived from a subject;
   detecting a level of pathogenic transcript variants of PSEN2 in the tissue sample; and
   based on detecting the level of pathogenic transcript variants of PSEN2, diagnosing the subject as having the tauopathy.
60. The method of embodiment 60, wherein the level of pathogenic transcript variants of PSEN2 includes a ratio of pathogenic transcript variants of PSEN2 per sample to wild-type transcripts of PSEN2 per sample.
61. The method of embodiment 60 or 61, wherein the pathogenic transcript variants of PSEN2 include a transcript with the inclusion of exon 9B.
62. The method of any of embodiments 60-62, wherein the pathogenic transcript variants of PSEN2 include a transcript with the inclusion of 77 bp of the intronic sequence prior to exon 6.
63. The method of any of embodiments 60-63, wherein the pathogenic transcript variants of PSEN2 include the sequence set forth in SEQ ID NO: 2, SEQ ID NO: 3, SEQ ID NO: 4, SEQ ID NO: 5, SEQ ID NO: 6, and/or SEQ ID NO: 7.
64. The method of any of embodiments 60-64, wherein the pathogenic transcript variants of PSEN2 include a sequence having 90% sequence identity to the sequence set forth in SEQ ID NO: 2, SEQ ID NO: 3, SEQ ID NO: 4, SEQ ID NO: 5, SEQ ID NO: 6, and/or SEQ ID NO: 7.
65. The method of any of embodiments 60-65, wherein detecting includes extracting RNA from the tissue sample; converting the RNA into cDNA; amplifying cDNA; and performing sequencing analysis.
66. The method of any of embodiments 60-66, wherein detecting the level of the pathogenic transcript variants of PSEN2 includes:
   quantifying PSEN2 reads per sample;
   quantifying select alternative splicing isoforms of PSEN2 reads per sample; and
   determining a percentage of select alternative splicing isoforms of PSEN2 compared to PSEN2 reads per sample.
67. The method of any of embodiments 60-67, wherein diagnosing the subject includes determining that the level of pathogenic transcript variants of PSEN2 is higher than a reference level.
68. The method of embodiment 68, wherein the reference level is the level of pathogenic transcript variants of PSEN2 in subjects without a neuropathologic disease.
69. The method of any of embodiments 60-69, wherein the tauopathy is sporadic Alzheimer's disease.

### EXPERIMENTAL EXAMPLES

### Example 1. Aberrant Splicing of PSEN2, but not PSEN1, in Individuals with Sporadic Alzheimer's Disease

In this example, an aim was to characterize full-length transcripts of PSEN1 and PSEN2 in brain tissue from individuals with familial and sporadic Alzheimer's disease as well as controls using PacBio Isoform Sequencing (Iso-Seq). Early-onset familial AD cases are caused in part by pathogenic variants in presenilin 1 (PSEN1) and presenilin 2 (PSEN2) and alternative splicing of these two genes has been implicated in both familial and sporadic AD. Targeted PacBio Iso-Seq was leveraged to characterize thousands of complete PSEN1 and PSEN2 transcripts in the prefrontal cortex of individuals with sporadic AD, familial AD (carrying PSEN1 and PSEN2 variants), and controls. PacBio Iso-Seq is a state-of-the-art technique that uses Single Molecule, Real-Time (SMRT) sequencing to completely sequence all targeted mRNAs up to 20 kb long (Tseng E, et al. Front Genet. 2019; 10:584.). The long-read capability of PacBio Iso-Seq is the only way to fully capture all exons in combination with pathogenic variants in PSEN1 (2,776 bp) and PSEN2 (2,249 bp) transcripts. Unlike short-read sequencing, it enables identification of complete novel transcripts and alternative splicing events, as well as quantify differences in transcript abundance between samples.

### Materials and Methods

Samples Used. The University of Washington Alzheimer's Disease Research Center (UW ADRC) Neuropathology Core provided post-mortem prefrontal cortex samples from the 20 cases listed in Table 1. All samples were collected following informed consent approved by the UW Institutional Review Board. Control individuals were recruited through the Adult Changes in Thought (ACT) study from Kaiser Permanente Washington (Kukull W A, et al. Arch Neurol. November 2002; 59(11):1737-46.), a population-based cohort study in which participants 65 years or older undergo cognitive screening every two years. Tissues utilized for this example were collected during rapid brain autopsy and flash frozen in liquid nitrogen or supercooled isopentane. Here, two controls, two sporadic AD, and two PSEN1 samples were frozen in isopentane.

TABLE 1
Samples used in study.

| | Age at | | | Cognitive | PSEN Variant, | APOE | BRAAK |
|---|---|---|---|---|---|---|---|
| Cohort | Death | Sex | PMI | Status | If Applicable | Status | Score |
| Control | 88 | F | 3.50 | No dementia | | 3/3 | III |
| Control | 90 | M | 5.00 | No dementia | | 3/3 | III |
| Control | 88 | F | 3.18 | No dementia | | 2/3 | II |
| Control | 90 | F | 4.50 | No dementia | | 2/3 | III |
| Control | 78 | M | 10.02 | No dementia | | 3/3 | 0 |
| Control | 83 | F | 2.67 | No dementia | | 3/3 | III |
| PSEN | 43 | M | 2.50 | Dementia | PSEN1, I143T | 3/4 | VI |
| PSEN | 55 | M | 3.12 | Dementia | PSEN1, S212Y | 2/4 | VI |
| PSEN | 58 | M | 6.33 | Dementia | PSEN2, N141I | 3/3 | VI |
| PSEN | 73 | M | 5.50 | Dementia | PSEN2, N141I | | VI |
| PSEN | 67 | F | 10.18 | Dementia | PSEN2, K115fsX | 3/3 | VI |
| PSEN | 40 | F | 4.65 | Dementia | PSEN1, V272A | | VI |
| PSEN | 38 | M | 9.85 | Dementia | PSEN1, M146L | | VI |
| Sporadic | 90+ | M | 6.00 | Dementia | | 3/4 | VI |
| Sporadic | 90+ | F | 4.50 | Dementia | | 3/3 | V |
| Sporadic | 88 | F | 3.33 | Dementia | | 3/3 | VI |
| Sporadic | 84 | F | 4.47 | Dementia | | 2/4 | V |
| Sporadic | 90+ | F | 5.50 | Dementia | | 3/4 | VI |
| Sporadic | 81 | F | 4.50 | Dementia | | 3/4 | VI |
| Sporadic | 60 | M | 8.68 | Dementia | | | VI |

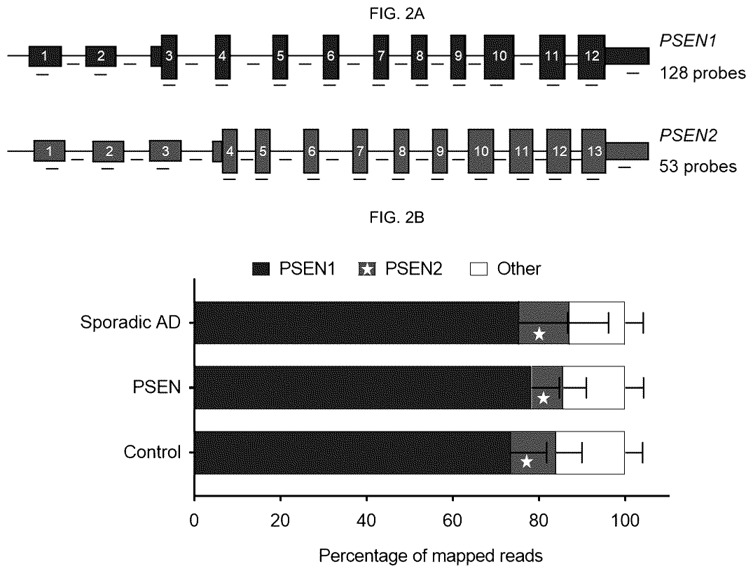

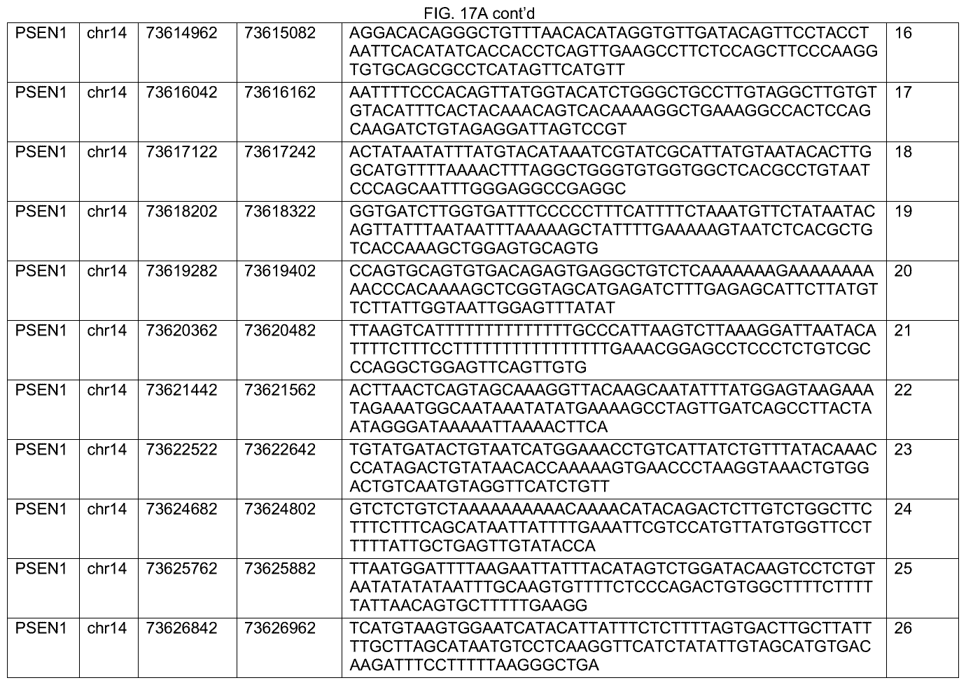

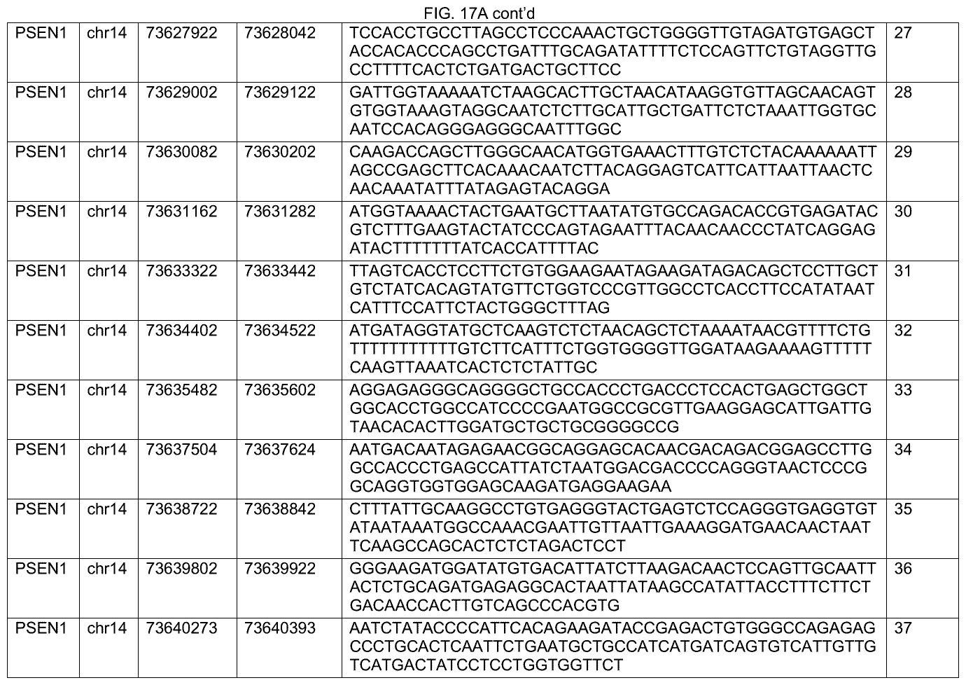

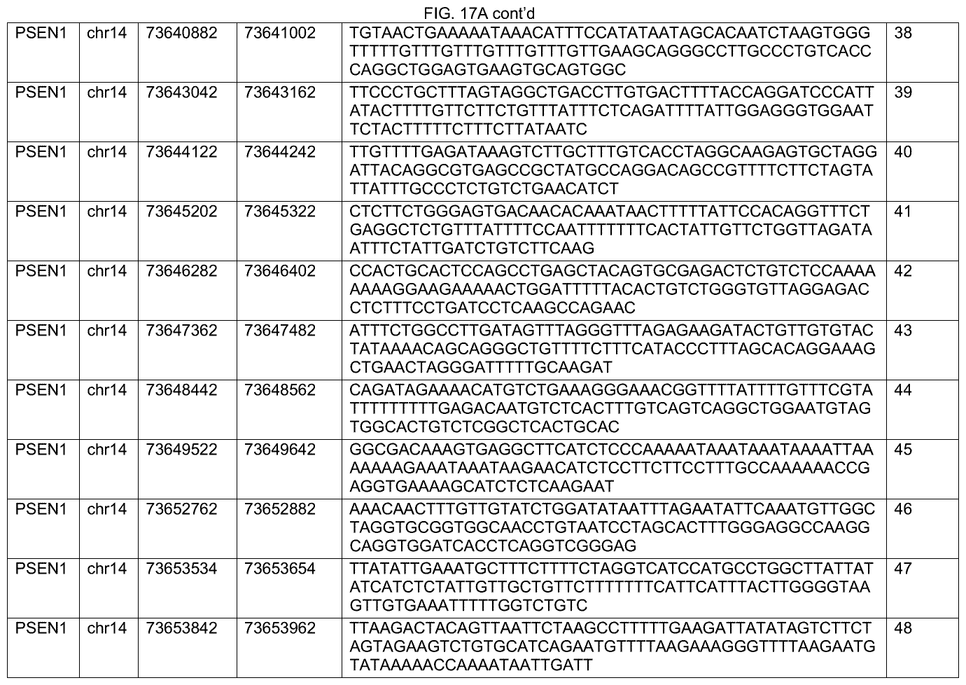

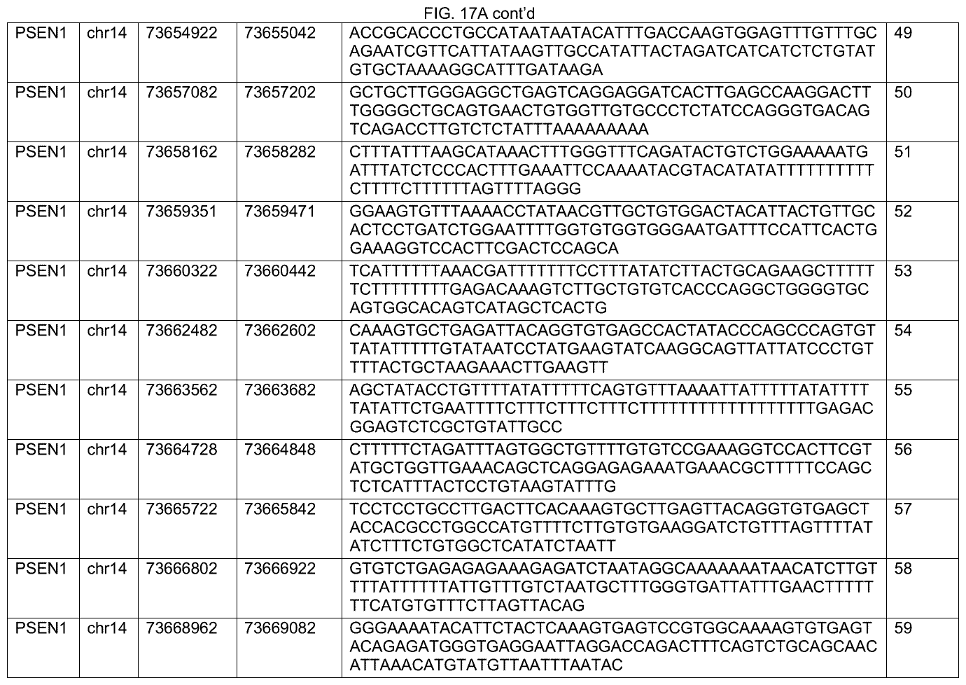

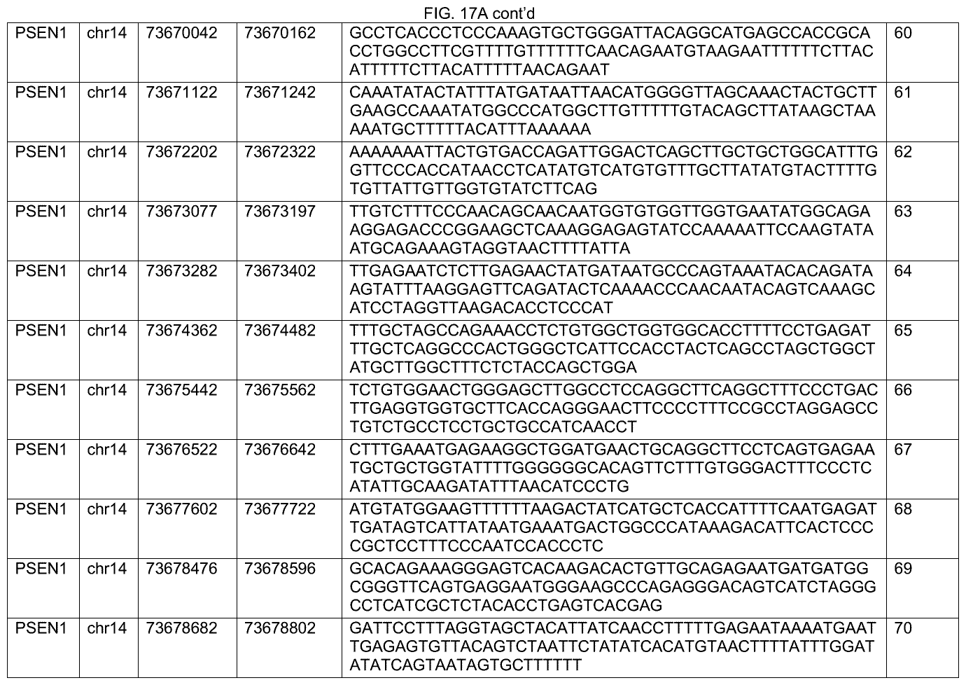

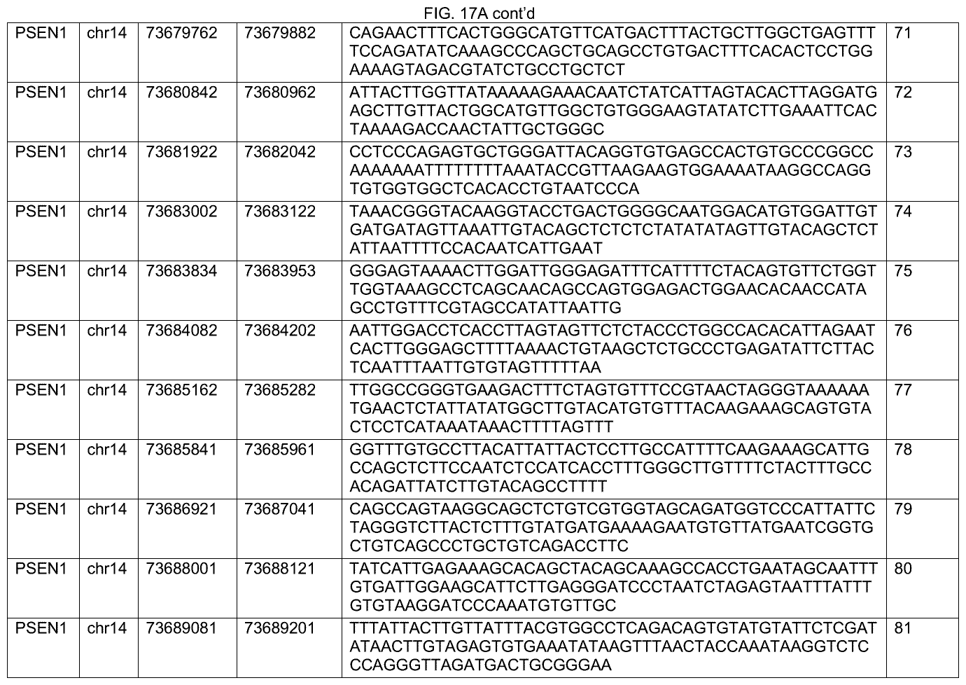

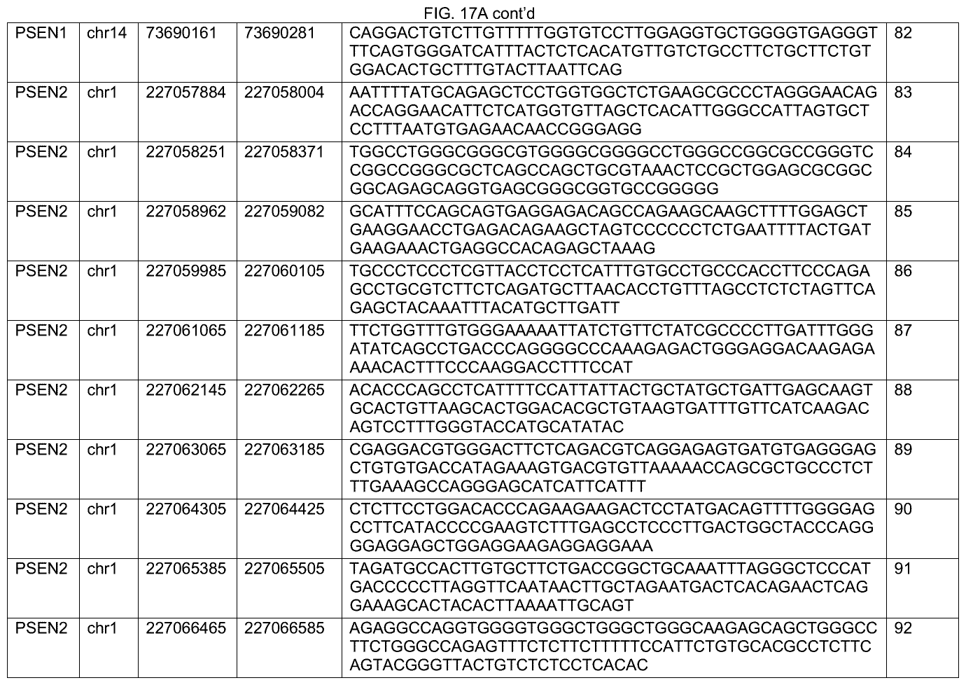

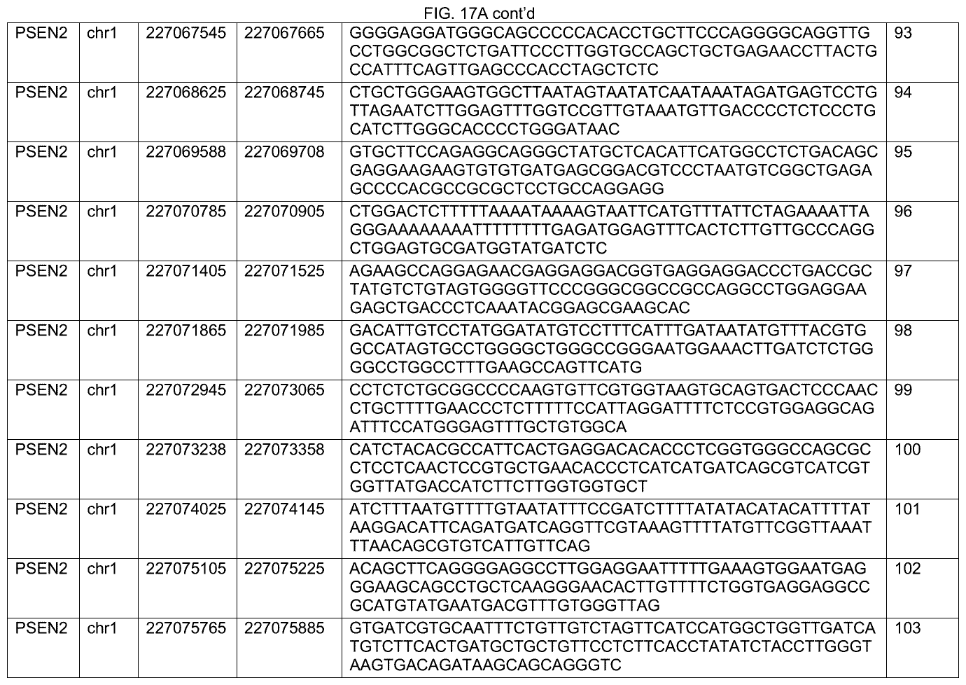

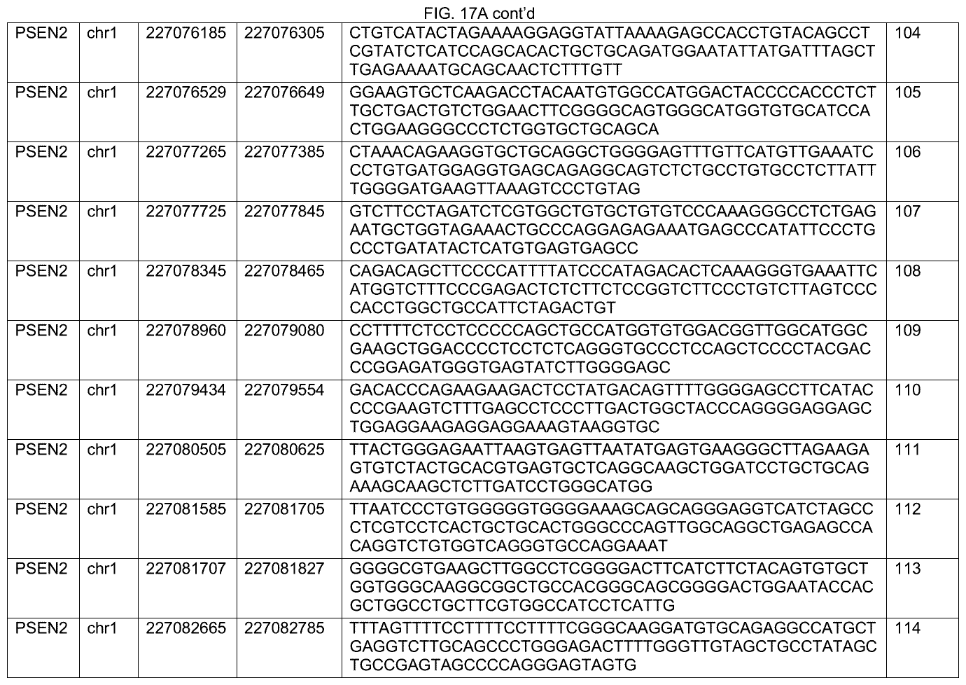

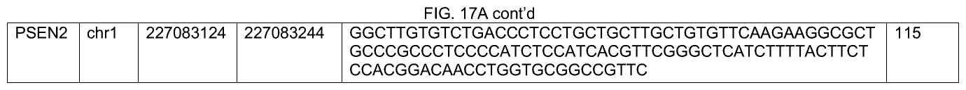

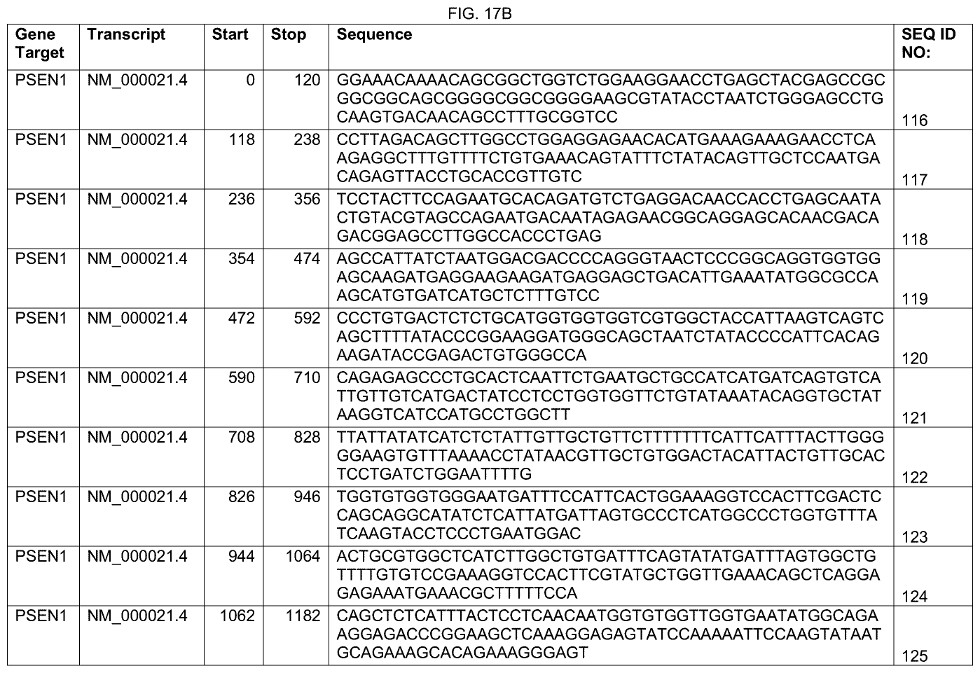

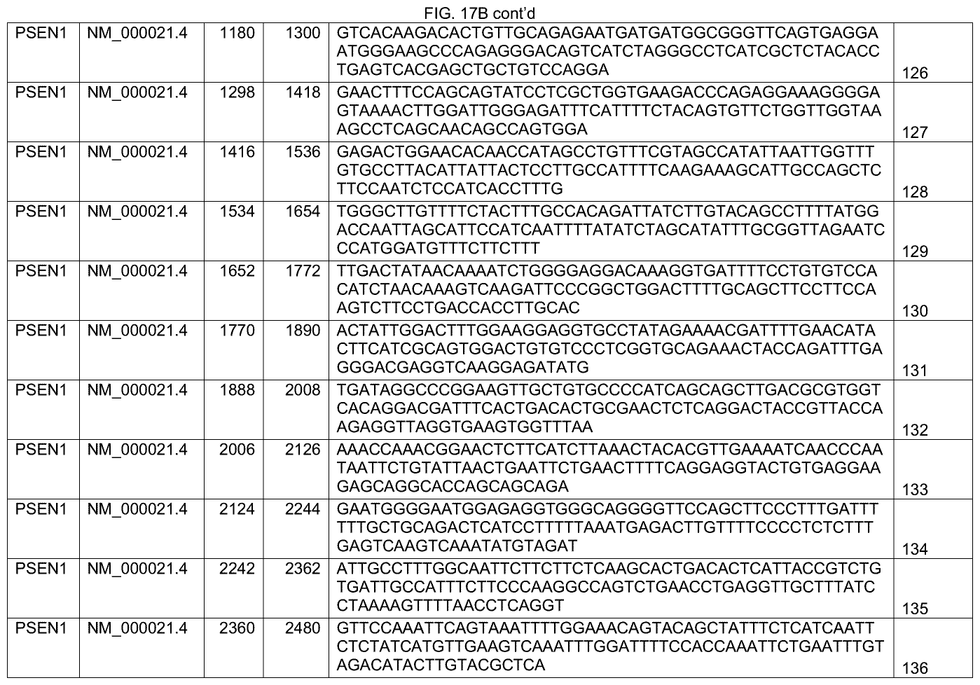

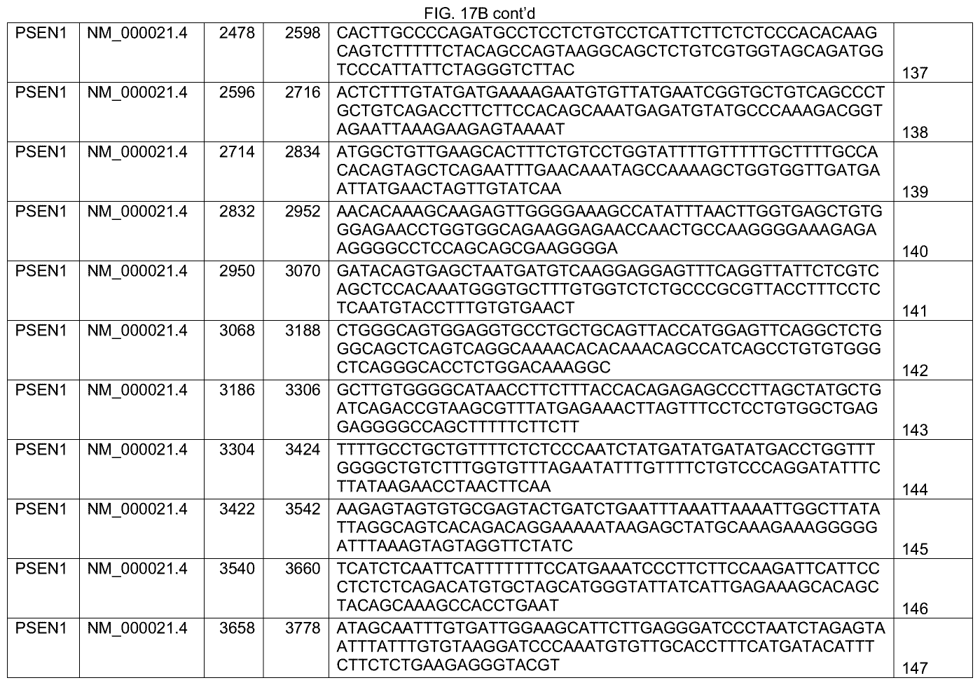

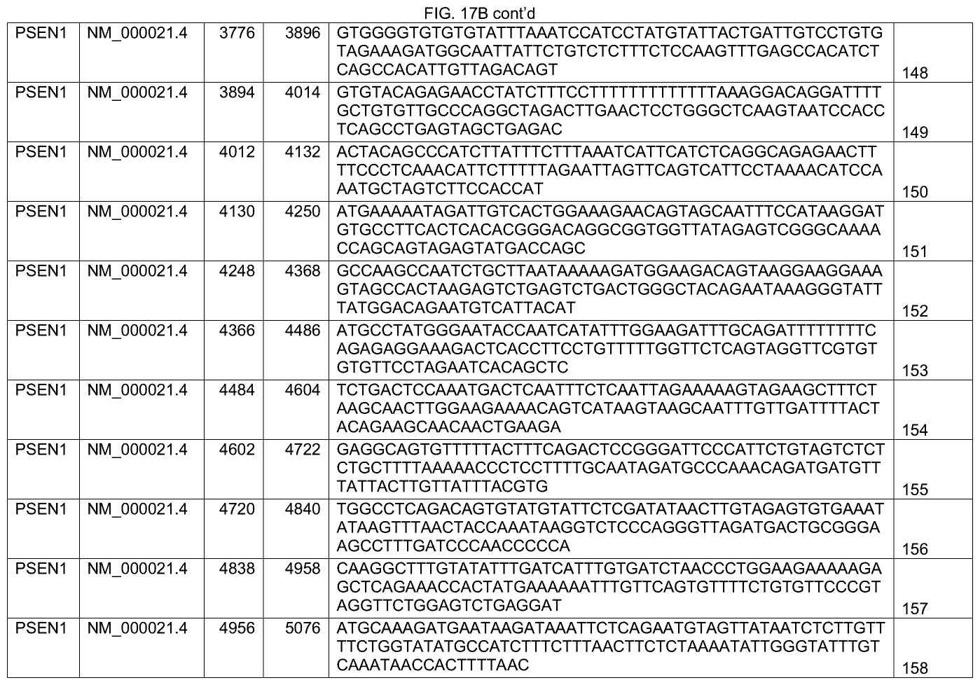

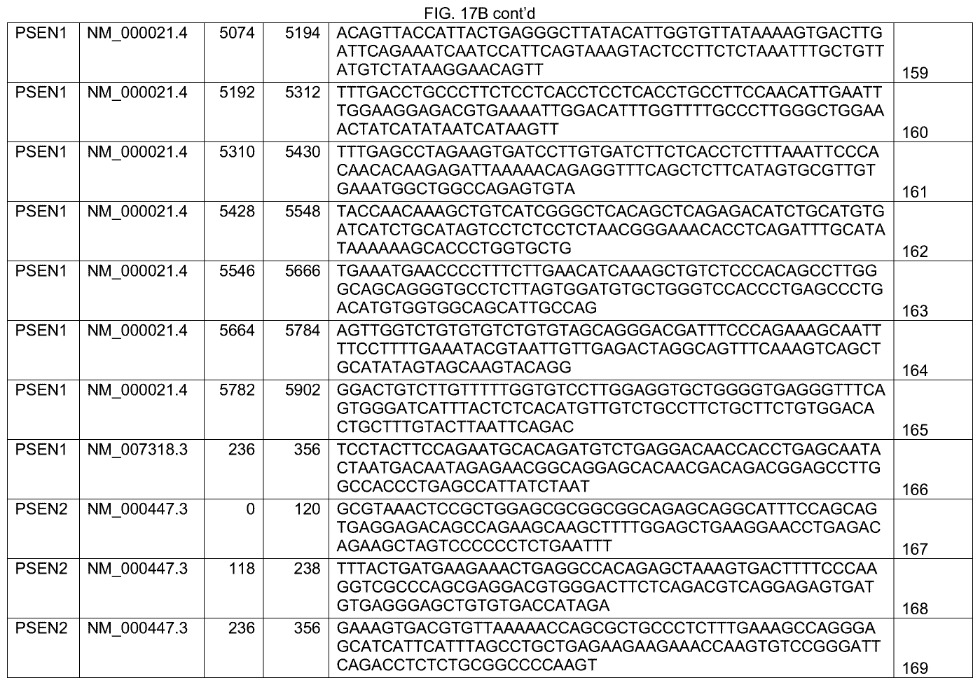

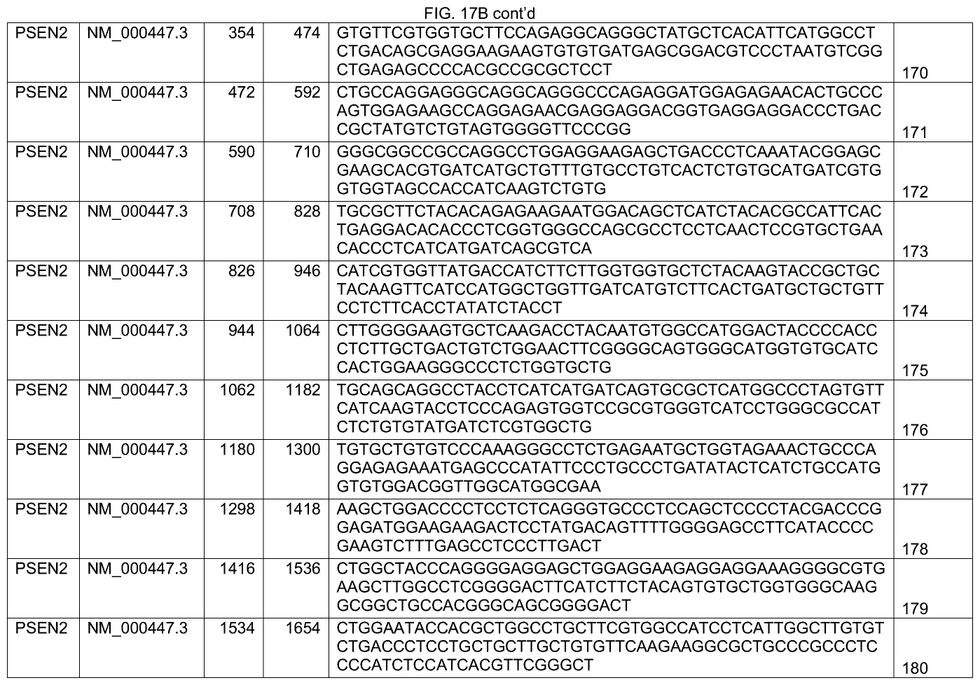

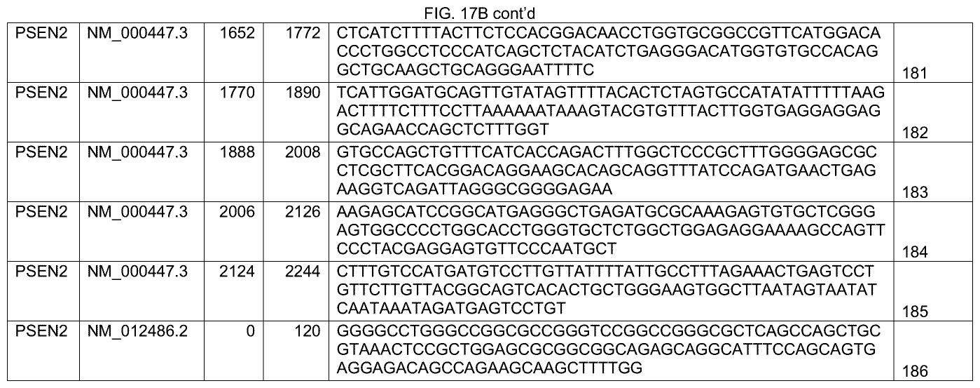

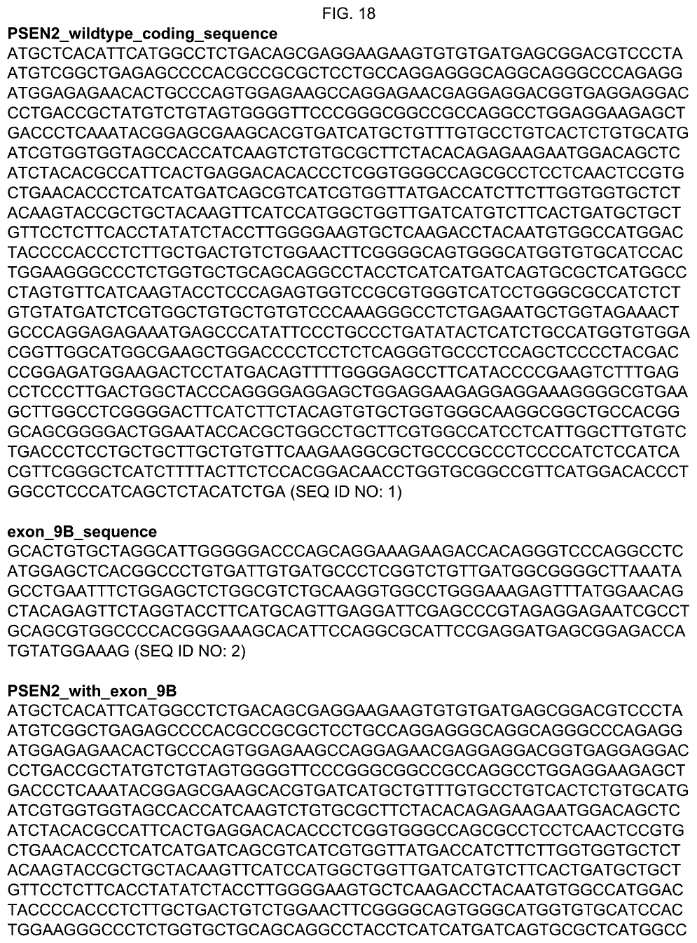

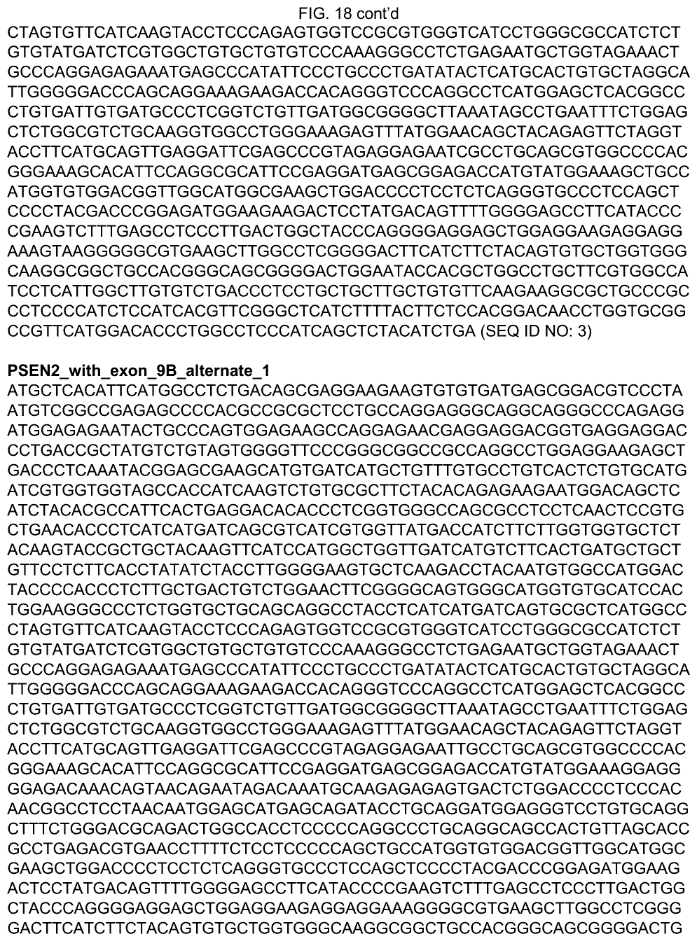

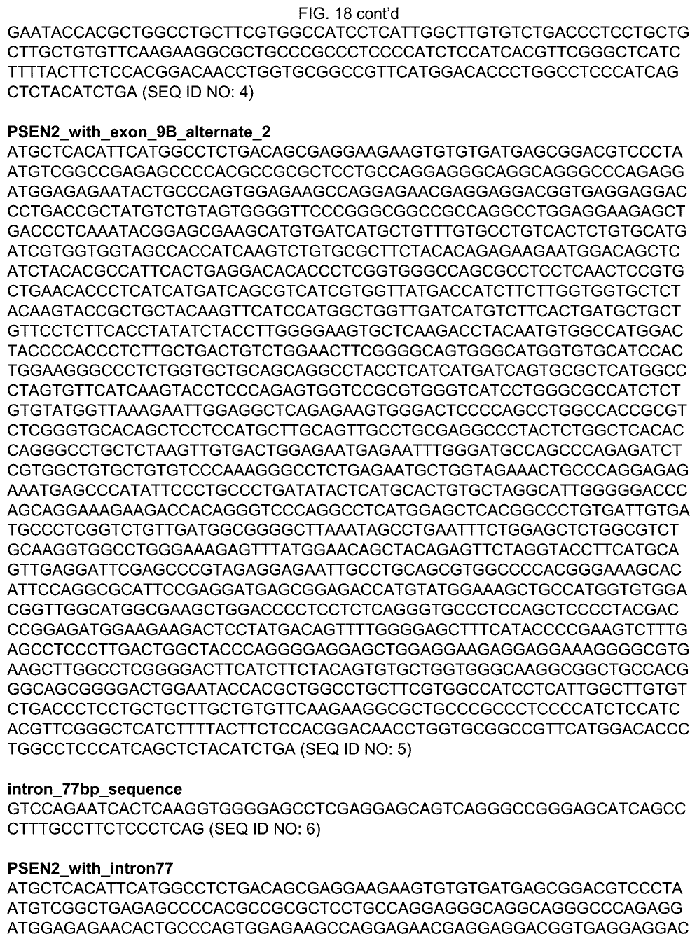

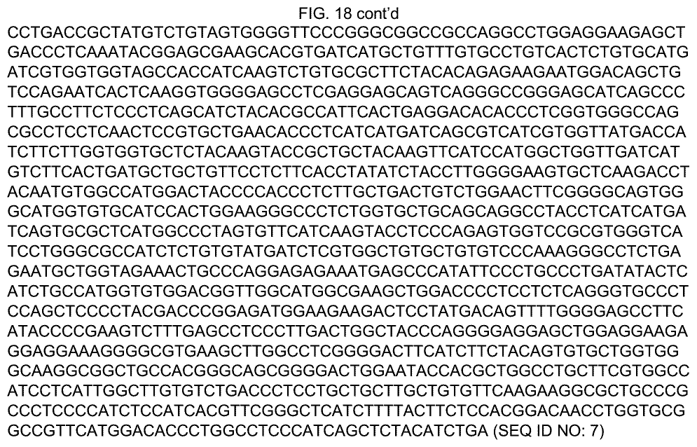

RNA was extracted from brain tissue samples using the RNeasy Lipid Tissue Mini Kit (Qiagen). 300 ng of RNA was then converted to cDNA, hybridized, and prepared for Iso-Seq following a protocol from PacBio called “Iso-Seq Express Capture Using IDT xGen Lockdown Probes.” Briefly, RNA was converted into cDNA and amplified using a combination of the NEBNext Single Cell/Low Input cDNA Synthesis & Amplification Module (New England Biolabs), the Iso-Seq Express Oligo Kit (PacBio), and barcoded primers (found in Appendix 3 of the PacBio protocol). RNA integrity number (RIN) scores for each sample varied, based on the quality of the original tissue. Samples were then pooled into two groups based on higher or lower RIN scores (researchers were blinded to sample identity), and these two pools were hybridized to a custom panel of xGen Lockdown Probes (Integrated DNA Technologies) using the xGen Lockdown Hybridization and Wash Kit (Integrated DNA Technologies). In total, the probe panel contained 181 probes tiling both exonic and intronic regions of PSEN1 (128 probes) and PSEN2 (53 probes) at equal intervals (FIG. 2A, FIGS. 17A, 17B). After 15 cycles of amplification of the captured cDNA, pools were combined (after confirming that both DNA pools had similar quality control scores) and submitted to University of Washington PacBio Sequencing Services for library preparation and running on PacBio Sequel II for 1 SMRT Cell 8M with 30-hour movie time, using SQIIv2.0 polymerase and v2.0 sequencing chemistry.

Iso-Seq Analysis. Full-length transcript information was received in FASTQ files, which were initially assessed using the standard Iso-Seq Analysis pipeline in SMRT Link (PacBio, version 10.1.0.119588). Briefly, barcoded reads were sorted, and barcodes and poly-A sequences were clipped using the Lima SMRT Analysis tool (version 2.2.0). Reads with a quality score of 20 or greater were mapped to the human GRCh38 genome using pbmm2 (version 1.3.0) and minimap2 (version 2.23) to generate a set of BAM files.

The number of reads per sample were counted with a transcript origin in PSEN1 (GRCh38 co-ordinates chr14:73,136,507-73,223,691) and PSEN2 (chr1:226,870,616-226,896,098). Of these, the number of reads were quantified that contained both canonical and alternative splice isoforms. To obtain total reads, the number of reads were counted that mapped to transcript coordinates. To ascertain full-length PSEN1 and PSEN2 transcripts, reads that contained the expected start and stop codon sequences were extracted. For uniquely mapped reads, all identical isoforms were counted as one.

RNA Editing Analysis. Iso-Seq reads that mapped to the AluJb (chr1:226896582-226896815) element in the PSEN2 3′UTR were assessed for the number of guanine residues at each adenine position. The percentage of total A-to-G changes was then quantified.

RNA-Seq Analysis. Mayo Clinic sample RNA-seq bam files from the cerebellum and temporal cortex were downloaded from Accelerating Medicines Partnership Program for Alzheimer's Disease (AMP-AD), Synapse ID syn5550404. Percent splice inclusion of alternative splice products was measured by calculating junction spanning reads across canonical and alternative splice junctions, confirmed by quantification from Sashimi plots generated by Integrated Genomics Viewer. Due to increased read depth at these positions, RNA editing was calculated across both AluJb (chr1:226896582-226896815) and AluY (chr1:226898626-226898913) elements in the PSEN2 3′UTR.

Statistical Analysis. Statistical analyses were performed using Prism version 9.2.0 (GraphPad Software). The Shapiro-Welk test (n<8)) or D'Agostino-Pearson omnibus K2 test (n >8) was first used to determine whether data were normally distributed. A One-Way ANOVA was used to compare groups of more than two with parametric distribution. If the ANOVA gave P<0.05, this analysis was then followed by Tukey's multiple comparisons. To analyze correlations, a Spearman correlation coefficient was used for nonparametric data, and a Pearson correlation coefficient was used for parametric data.

Data availability. RNA sequence reads from the Mayo Clinic are available from synapse.org with accession number syn5550404.

Results. To observe PSEN1 and PSEN2 transcripts in individuals with Alzheimer's disease and controls, cDNA was first prepared from 20 samples. These samples were composed of prefrontal cortex tissues, from six non-demented donors with low or absent Alzheimer's disease neuropathologic change (referred to as “controls”), seven individuals with both familial Alzheimer's disease and a PSEN1 or PSEN2 variant, and seven individuals with neuropathologically confirmed sporadic Alzheimer's disease (Table 1). Of the seven samples from individuals with PSEN1 or PSEN2 variants, four had PSEN1 variants (all different), and three had PSEN2 variants, one of which was the K115Efs variant, and two of which were N141I variants. Together, these samples are referred to as “PSEN variant carriers.” As noted later in this example, it was observed that one of the sporadic Alzheimer's disease samples carried a PSEN1 variant of unknown significance (VUS), and this sample was continued to be considered sporadic, based on the known literature of this VUS (the individual's late age of death at >90 years old also supports this decision). These samples were all obtained within a post-mortem interval of less than 12 hours. Controls and sporadic Alzheimer's disease samples were reasonably age- and sex-matched. Due to the early-onset nature of familial Alzheimer's disease, age-matched controls could not be obtained for the PSEN variant carriers (Table 1).

PSEN1- and PSEN2-specific transcripts were obtained from the cDNA by hybridization capture, and then submitted these samples for SMRT sequencing. The use of hybridization probes were favored over PCR amplification of transcripts to ensure that it would be possible to observe all alternative splice isoforms, including truncated isoforms. The hybridization probes corresponded to both coding sequence and genomic region (FIGS. 17A, 17B), so that potential intron retention products could be identified. The number of probes used per gene and a schematic of probe location is provided in FIG. 2A. A step in cDNA conversion involving selection for polyA tails suggests that the findings here reflect transcripts that have undergone mRNA processing.

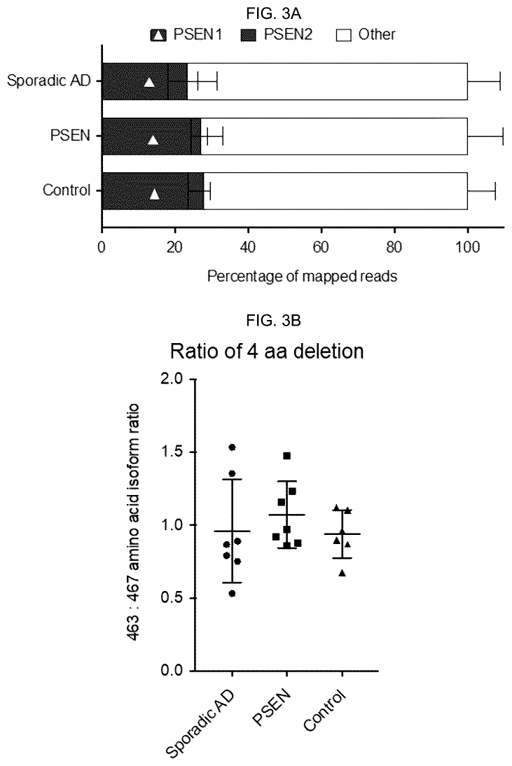

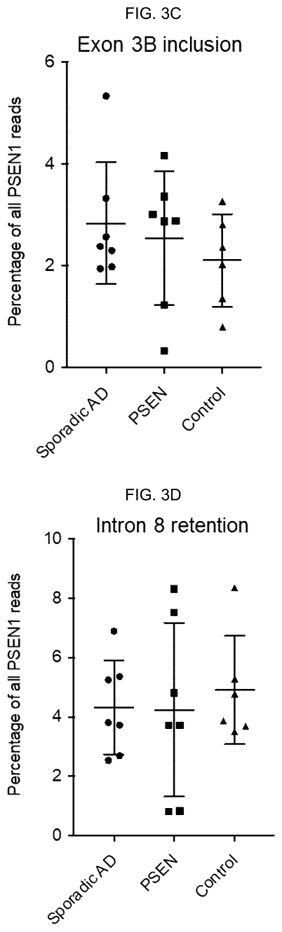

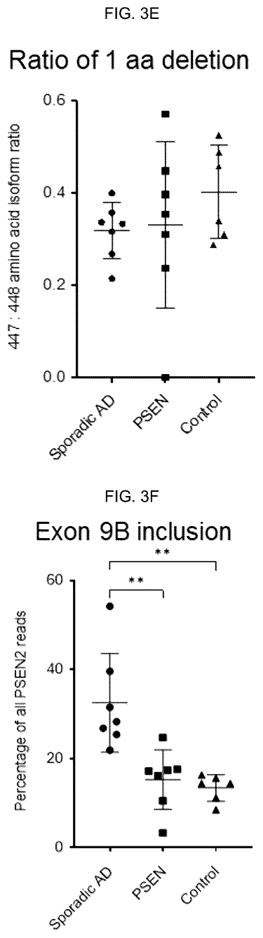

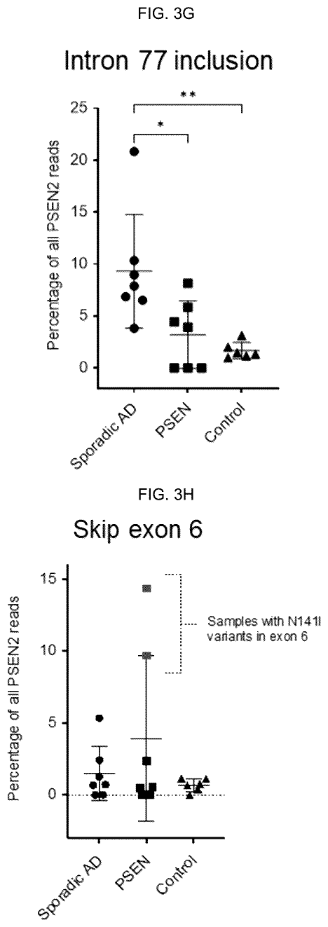

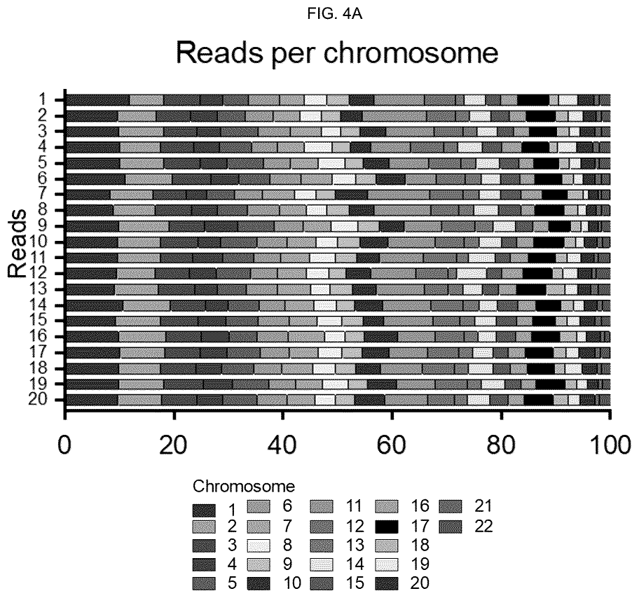

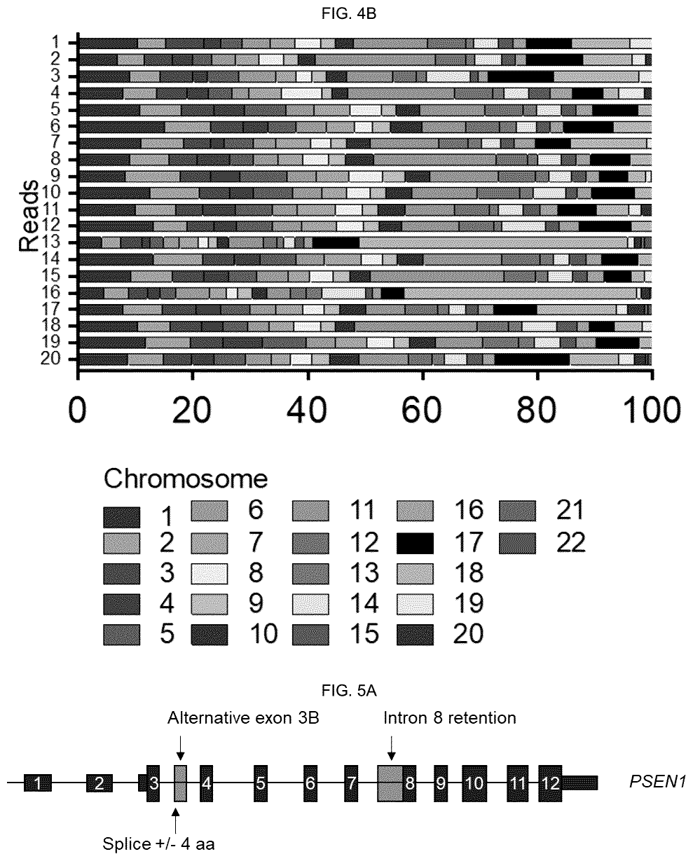

Alignment to PSEN1 and PSEN2. On average, 81,034+/−36,374 mapped reads per sample (range: 28,444-167,125) were obtained. Of these, 75.8+/−8.68% mapped to PSEN1 (range: 53.8-86.1%) and 9.86+/−6.91% mapped to PSEN2 (range: 2.69-31.5%). Values were not significantly different across cohorts (FIG. 2B; P=0.62 for PSEN1 and P=0.53 for PSEN2, One-Way ANOVA). Analysis of uniquely mapped reads instead of total reads led to similar results (FIGS. 3A-3H). The higher mapping rate for PSEN1 may reflect an increased abundance of PSEN1 in the frontal cortex or could be due to the higher number of probes used for PSEN1 mRNA versus PSEN2 mRNA (FIG. 2A). The reads that did not map to PSEN1 or PSEN2 were evenly distributed throughout the genome, with no evidence for biased enrichment of a particular gene when analyzing unique reads (FIG. 4A), and some preferential amplification of proteolipid protein 1 (PLP1) and myelin basic protein (MBP) in just two samples when analyzing total reads (FIG. 4B). The distribution of reads was comparable between cohorts.

PSEN1 isoforms. The generation of appropriately spliced and full-length PSEN1 was largely efficient, with 75% of reads encoding for an in-frame protein product. A minimum of 16,000 canonical full-length transcripts of PSEN1 that encoded for an in-frame protein product were found in each sample. Importantly, in the PSEN variant carriers, half of full-length transcripts of PSEN1 were observed to contain the pathogenic variant.

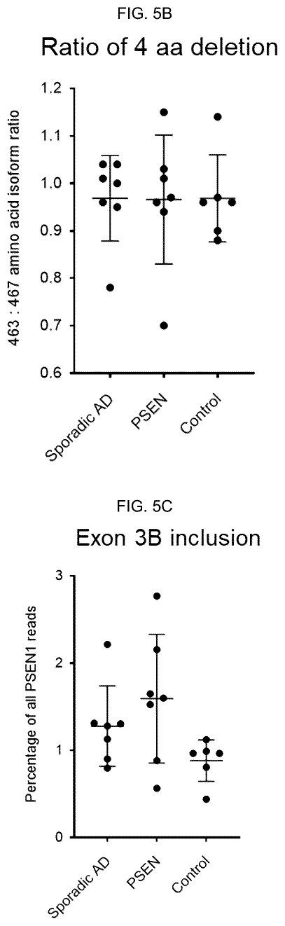

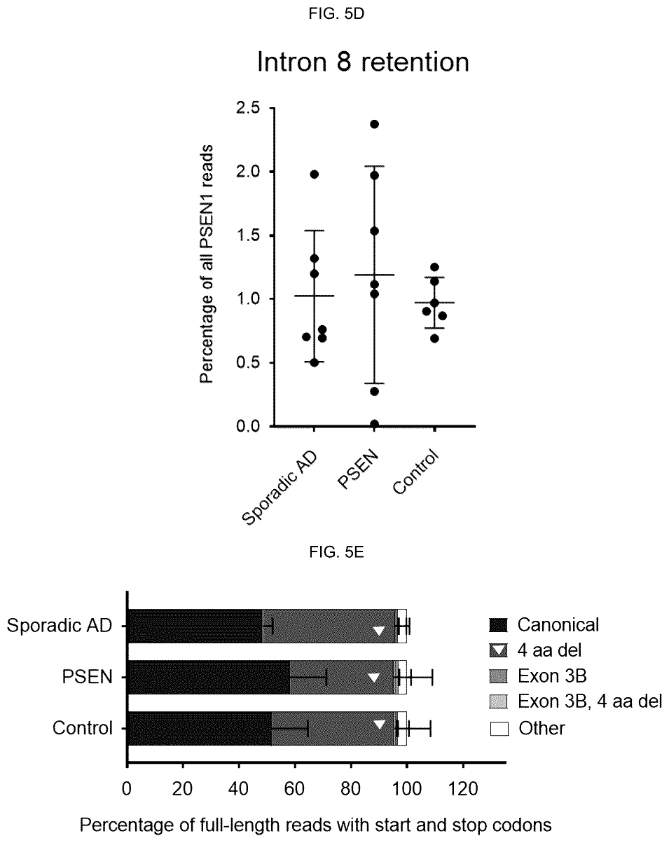

Two predominant splice isoforms were detected, corresponding to the omission or inclusion of 12 (bp; the amino acids VRSQ (SEQ ID NO: 187)) at the end of exon 3 of 12 (first coding exon) of the PSEN1 transcript (FIG. 5A). The abundance of the transcript without these 12 bp (463 aa; ENST00000357710.8) versus with these nucleotides (467 aa; ENST00000324501.10) was comparable across sporadic AD, PSEN variant carriers, and controls, with short and long isoforms each occurring at the same rate (FIG. 5B; P=1.00, One-Way ANOVA). Transcripts missing the 12 bp are thought to be the alternative transcripts, as these nucleotides are conserved in PSEN2 transcripts.

The third most common isoform from the analysis, however, was not annotated. This isoform of PSEN1 included a 130 bp alternatively spliced exon in intron 3 (GRCh38 chr14:73,160,009-73,160,138) (FIG. 5A). The extra exon (exon 3B) is composed of portions of AluJb and L1MB2 repeat elements. Despite introducing a frameshift and premature termination codon at amino acid 61, the transcript continues through to the 12^{th} and last exon of PSEN1. If an internal methionine start codon is used, the N-terminally truncated protein product would be 384 amino acids long. The inclusion of exon 3B remained the same between controls, PSEN variant carriers, and sporadic Alzheimer's disease samples (FIG. 5C; P=0.084, One-Way ANOVA). One of the next most abundant isoforms contains an alternative splice acceptor site in intron 8 that leads to an intron retention product and is present in 1% of reads for all samples. Again, this number remained the same across all three cohorts (FIG. 5D; P=0.79, One-Way ANOVA). All full-length transcripts carrying these alternative splice events are quantified as a percentage of total full-length PSEN1 isoforms observed across cohorts in FIG. 5E. Analysis of uniquely mapped reads gave similar results for all PSEN1 isoforms (FIGS. 4A, 4B).

In one sample annotated as sporadic, a 3 bp deletion in exon 4 of PSEN1 (p.Asp40del; GRCh38 co-ordinates chr14:73,170,825-73,170,827) was identified. This 3 bp deletion has been previously observed in Alzheimer's disease cases (Nicolas G, et al. Eur J Hum Genet. May 2016; 24(5):710-6.; Nygaard H B, et al. Am J Alzheimers Dis Other Demen. August 2014; 29(5):433-5.) and is present in gnomAD (v3.1.2) in 18 individuals (allele frequency of 0.0001183), indicating that it is currently a VUS. In a recent systematic study, this variant was deemed a risk factor for AD (Hsu S, et al. Neurobiology of disease. June 2020; 139:104817.).

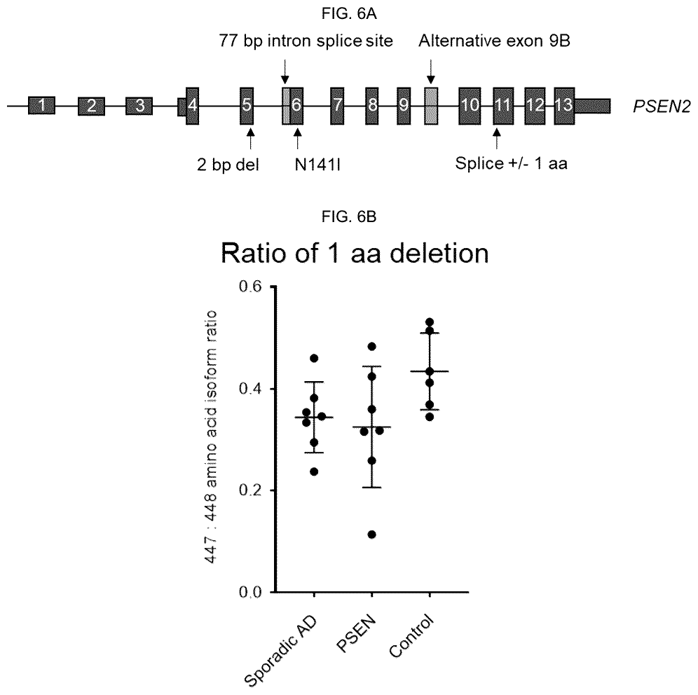

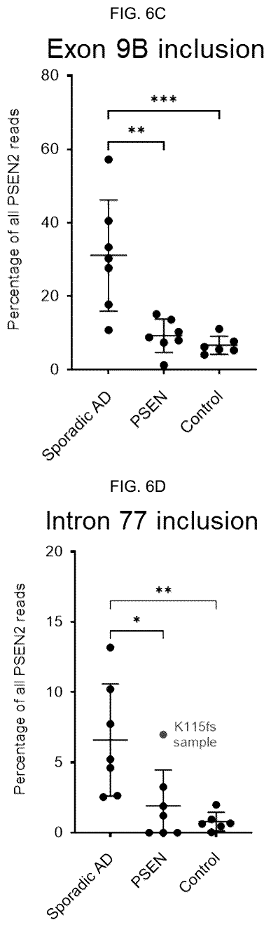

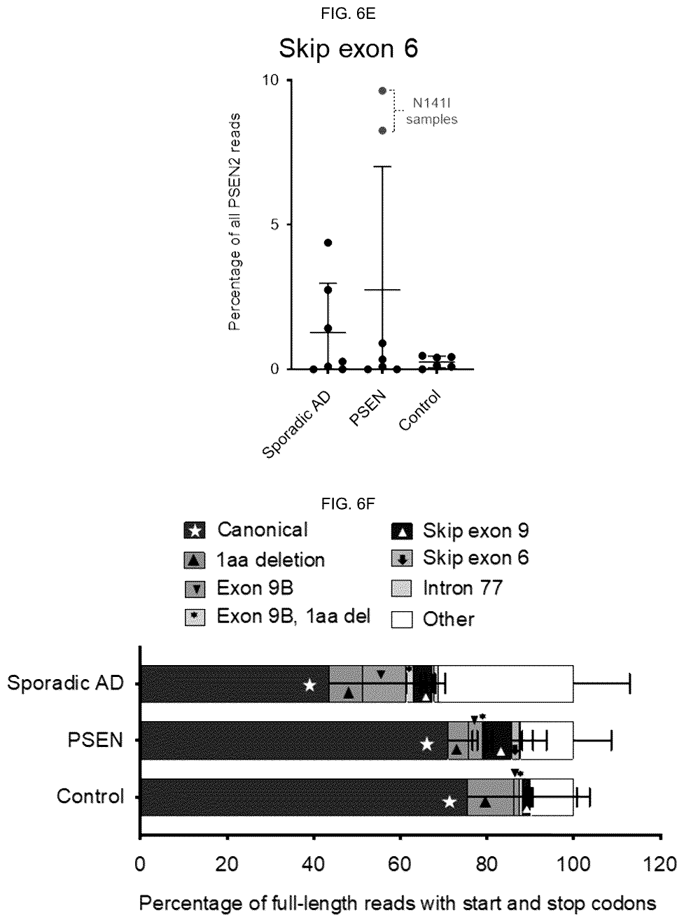

PSEN2 isoforms. Alignment of Iso-Seq reads to PSEN2 revealed a more heterogeneous population of transcripts than those observed for PSEN1 (FIG. 6A). The generation of an appropriately spliced and full-length PSEN2 was slightly less efficient than for PSEN1, with 62% of reads encoding for an in-frame protein product. At least 882 canonical full-length transcripts of PSEN2 were found in each sample. Like PSEN1, full-length transcripts contained both wildtype and pathogenic variant alleles for each of the PSEN variant carriers.

Again, after the canonical PSEN2 transcript, the second most abundant transcript that spanned from start to stop codons was one that contained an alternative splice site—an alternative acceptor site in this instance—at the beginning of exon 11 of 13, which resulted in the removal of 3 bp (1 aa) from the total protein product. Sporadic AD samples, controls, and PSEN variant carriers all had similar levels of this isoform, with the short isoform occurring less often than the long isoform (FIG. 6B; P=0.11, One-Way ANOVA).

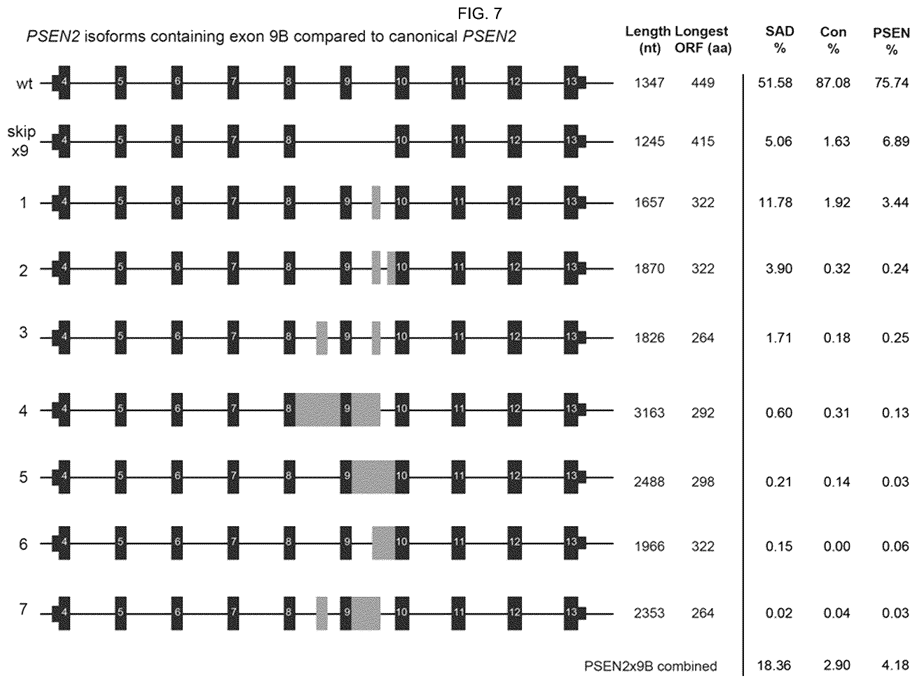

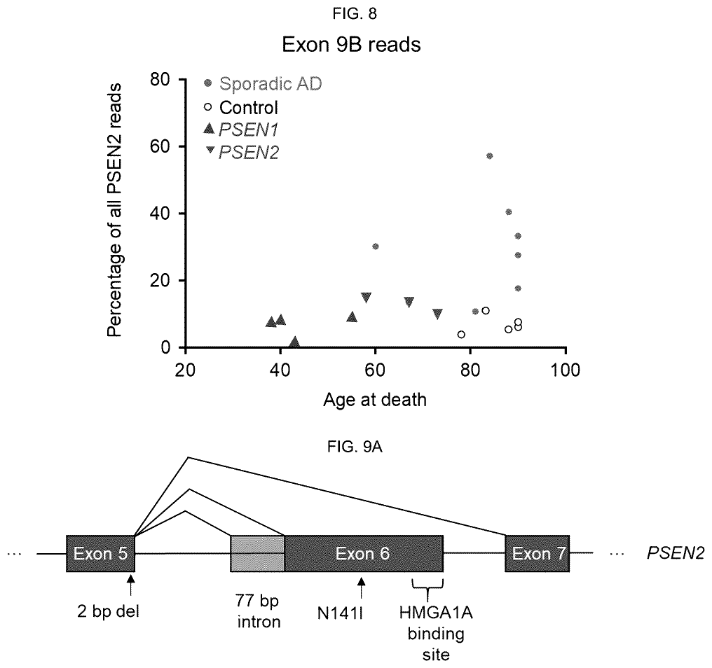

Also like PSEN1, the third most abundant transcript was non-productive (i.e., a premature stop codon is introduced). A 1657 bp transcript was detected in 4.8% of full-length reads across all samples. This alternative exon appears between exons 9 and 10, herein referred to as exon 9B (FIG. 6A). The exon is 310 bp, flanked by canonical splice sites, contains stop codons in all three reading frames, and is predicted to produce a protein truncated at 321 amino acids. Reads mapping to the exon 9B region accounted for over 50% of total PSEN2 reads for one sporadic sample and were significantly more abundant in sporadic AD than in controls (P=0.0007) or PSEN variant carriers (P=0.0013), while PSEN variant carriers exhibited similar levels as controls (FIG. 6C; P=0.88, One-Way ANOVA followed by Tukey's multiple comparisons). A known isoform of PSEN2 (ENST00000487450.1) lists the presence of exon 9B, though mRNA abundance stemming from this isoform is low based on GTEx data. Several other transcript isoforms were observed that contained exon 9B in combination with intron retention and alternative splice products present in introns 8 and 9, many of which were enriched in sporadic AD (FIG. 7). Exon 9B inclusion was not correlated with age at death in sporadic or any other cases (FIG. 8; r=0.24, p (two-tailed)=0.30, Spearman correlation).

A fourth PSEN2 isoform was also detected corresponding to the inclusion of 77 bp of intronic sequence prior to exon 6 (FIG. 6A). The presence of this 77 bp intron product was previously identified in the context of a 2 bp deletion (De Strooper B. EMBO Rep. February 2007; 8(2):141-6.). This transcript is a full-length mRNA including start and stop codons and all exons in-between, but due to a frame shift, is not predicted to form a full-length protein product. Again, sporadic AD samples had a significantly higher abundance of this 77 bp intron product as compared to controls (P=0.0050) or PSEN variant carriers (P=0.017), while PSEN variant carriers exhibited similar levels as controls (FIG. 6D; P=0.76, One-Way ANOVA followed by Tukey's multiple comparisons). Average percentages were 6.59% for sporadic AD, 1.91% in PSEN (though three samples had zero reads) and 0.79% in controls. The highest level of the 77 bp intron product in the PSEN variant carriers was observed in the PSEN2 2 bp deletion (PSEN2 K115fs; highlighted in FIG. 6D) (De Strooper B. EMBO Rep. February 2007; 8(2):141-6.).

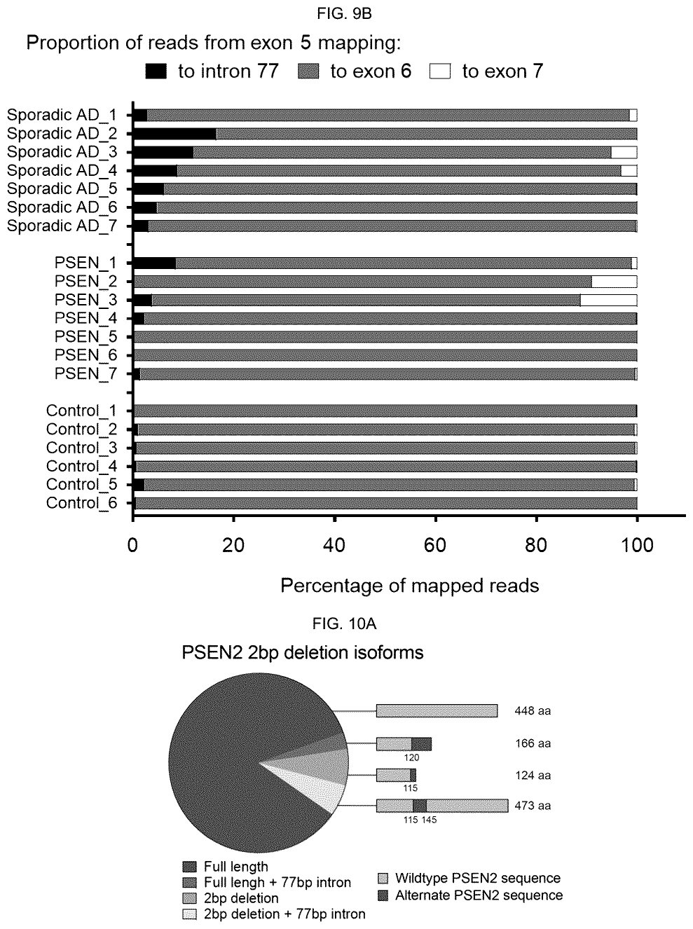

Exon 6 skipped products are not a common feature of PSEN2 Iso-Seq data. An exon 6 skipped product, termed PS2V, is the subject of several previous reports (Sato N, et al. J Neurochem. June 1999; 72(6):2498-505.; Manabe T, et al. Cell Death Differ. June 2003; 10(6):698-708.; Moussavi Nik S H, et al. J Alzheimers Dis. 2021; 80(4):1479-1489.; Smith M J, et al. Brain Res Mol Brain Res. Aug. 23, 2004; 127(1-2):128-35.). This exon skipping is induced by hypoxia and mediated by binding of HMGA1 (Moussavi Nik S H, et al. Exp Cell Res. Jul. 1 2011; 317(11):1503-12.; Manabe T, et al. Cell Death Differ. June 2003; 10(6):698-708.). Here, few examples were found of reads that skip exon 6, including in the 2 bp deletion sample, where deletion of exon 6 would restore full length protein production (minus 48 aa) (Braggin J E, et al. Ann Clin Transl Neurol. April 2019; 6(4):762-777.). Abundance of transcripts exhibiting exon 6 skipping remained the same across cohorts (FIG. 6E; P=0.28, One-Way ANOVA). A notable exception to this lack of PS2V was in the two carriers of the PSEN2 N141I pathogenic variant. In each, 8.26-9.64% of all reads that mapped to PSEN2 contained a splice product skipping exon 6 (highlighted in FIG. 6E). Interestingly, amino acid 141 is in the middle of exon 6, suggesting that the nucleotide variant may influence alternative splicing of exon 6 (FIG. 6A). The consensus sequence of HMGA1 is CUGCUACAAG (SEQ ID NO: 188) at the end of exon 6 (amino acids 163-166), distal to the N141 codon (FIGS. 9A, 9B).

In one of the PSEN2 N141I carriers, a heterozygous position (rs1046240) was found in exon 5. Using this information, it could be deduced that all 479 reads that skipped exon 6 also contained the alternate allele at rs1046240 and therefore were in cis with the 1411 allele. For the second N141I individual, however, heterozygous positions were not identified in the mRNA that could be leveraged for a similar analysis.

Finally, all the full-length PSEN2 transcripts carrying these alternative splice events mentioned, along with a splice product that skips exon 9 but maintains the PSEN2 reading frame, are quantified as a percent of all full-length PSEN2 isoforms observed across cohorts in FIG. 6F. Notably, in contrast to PSEN1, the proportion of full-length reads is significantly lower in sporadic AD cases relative to controls (P=0.0055) or PSEN variant carriers (P=0.013, One-Way ANOVA followed by Tukey's multiple comparisons). Analysis of uniquely mapped reads led to similar results for all PSEN2 isoforms (FIGS. 4A, 4B).

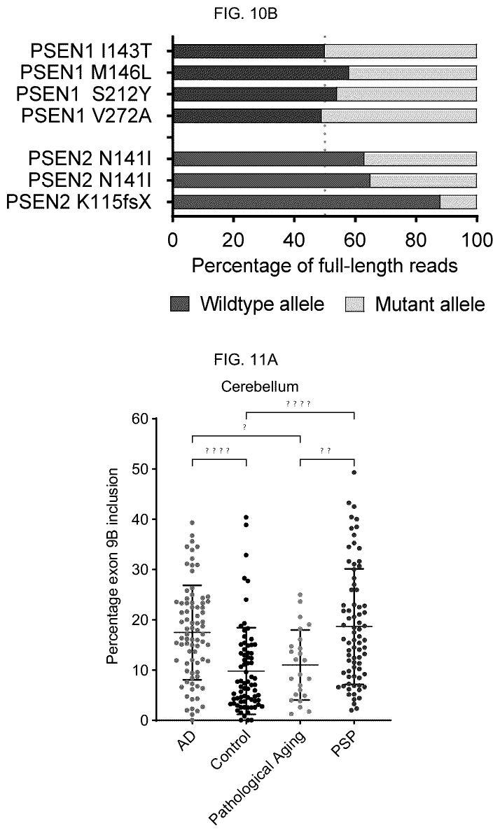

Allelic bias at PSEN1 and PSEN2 pathogenic variant positions. Long-read Iso-Seq also afforded the ability to examine allelic bias in PSEN variant carriers. An unresolved question from previous work was whether the 2 bp deletion could produce a full-length in-frame product in combination with the 77 bp insertion in intron 6. Aligning reads from this individual revealed that the wildtype allele accounted for 88% of the reads (2858 reads for full-length PSEN2 and 98 reads including the 77 bp intronic sequence). Of the 412 reads with a 2 bp deletion, half spliced to the canonical exon 6 junction and continued to the last exon of the transcript, which is predicted to yield a truncated 124 aa protein product (FIG. 10A). 190 additional reads, however, included the 77 bp product, generating a full-length transcript with 25 additional amino acids inserted after exon 5.

The PSEN2 2 bp deletion allele (i.e., the K115fs variant) only contributes to 12% of total PSEN2 transcripts, therefore some nonsense-mediated decay is likely active. The other two PSEN2 samples (both with the N141I variant) displayed only 35-37% of transcripts with pathogenic variants, further indicating that selective expression or increased stability of the wildtype allele may take place. For PSEN1, the pathogenic variant was present in transcripts in equal amounts, at 42-51% of the time (FIG. 10B).

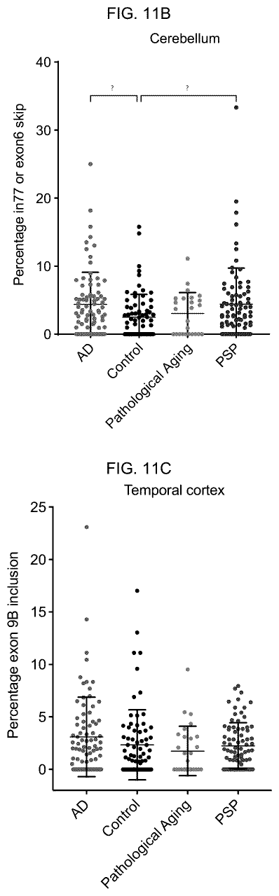

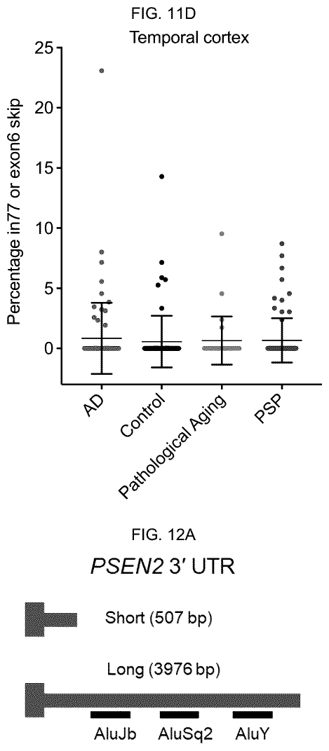

Confirmation of PSEN1 and PSEN2 alternative splicing events in independent RNA-seq datasets. To determine whether the PSEN2 exon 9B splice product is a prominent feature of AD, RNA-seq datasets made available through the AMP-AD were queried. It was determined that the exon 9B splice products were highest in the cerebellum from the Mayo Clinic (Allen M, et al. Sci Data. Oct. 11, 2016; 3:160089.), which corresponds to increased expression of PSEN2 in the cerebellum and cerebellar hemisphere relative to other brain regions based on GTEx data (Consortium G. Science. Sep. 11, 2020; 369(6509):1318-1330.). The Mayo Clinic dataset contains RNA-seq data from the cerebellum for 82 individuals with sporadic AD, 76 controls, 25 individuals with pathological aging (Aβ deposits but no cognitive impairment), and 81 individuals with progressive supranuclear palsy (PSP). Exon 9B was included 17.4% of the time on average in AD samples, significantly higher than in controls (9.81%, p<0.0001) and in the pathological aging cohort (11.0%, p=0.024) but not different than PSP samples (FIG. 11A; 18.6%, p=0.87, One-Way ANOVA followed by Tukey's multiple comparisons).

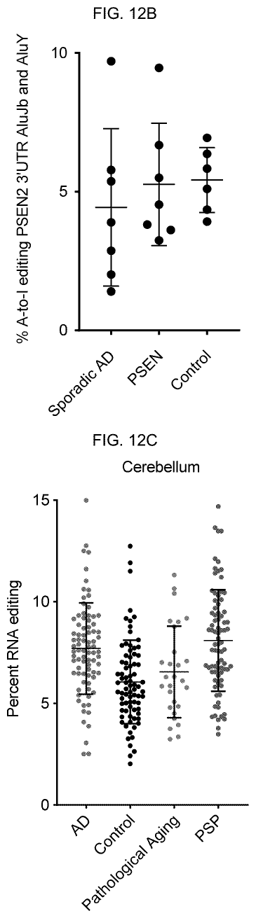

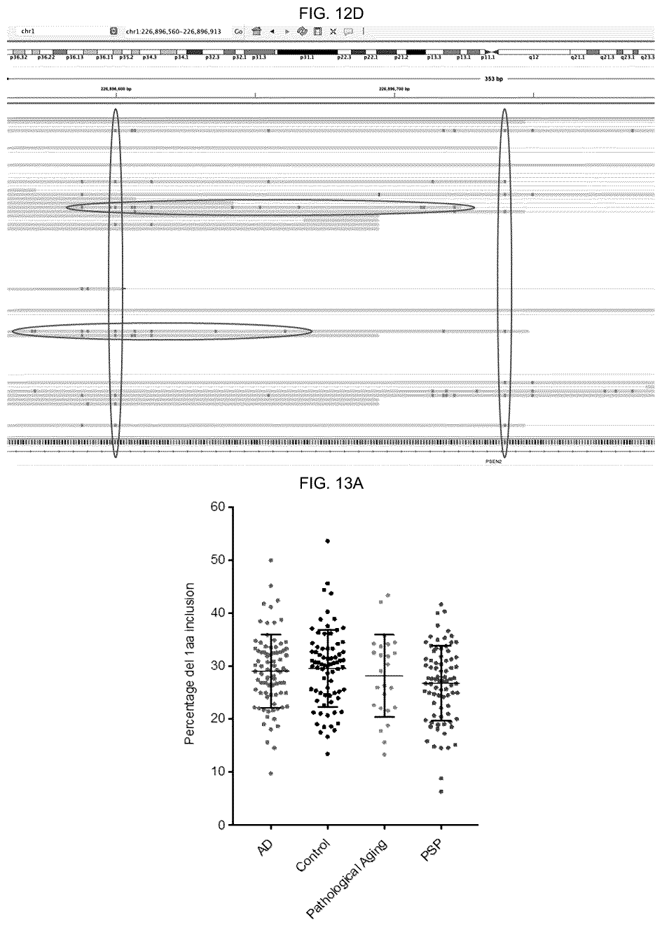

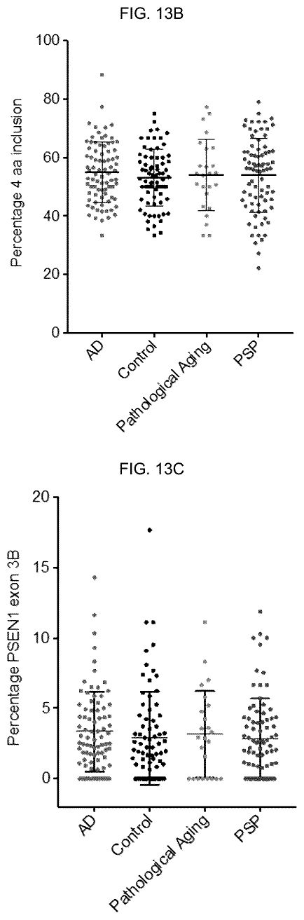

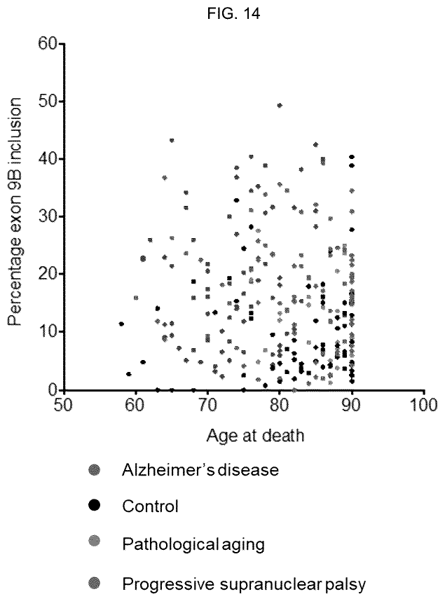

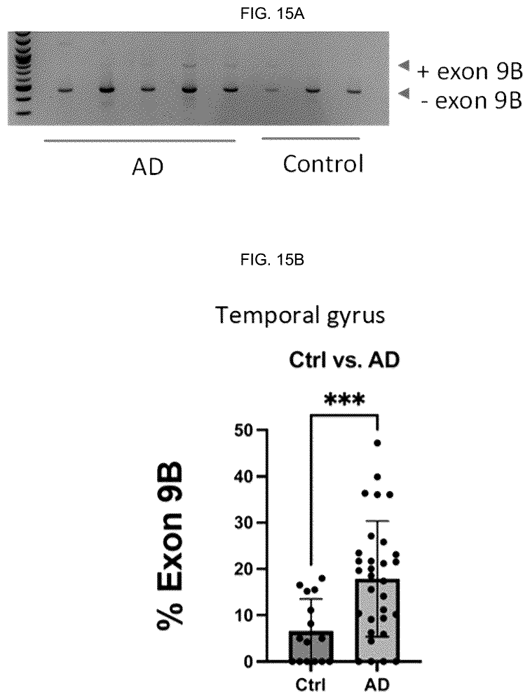

In addition, it was observed that both exon 6 skipping and 77 bp intron inclusion alternative splice events at exon 6. Alternative splicing at exon 6 was higher in AD samples versus controls (4.37% versus 2.52%; p=0.045), as well as in PSP versus controls (4.42% versus 2.52%; p=0.037), but not for AD versus pathological aging (p=0.54) or PSP (FIG. 12B; p=1.00, One-Way ANOVA followed by Tukey's multiple comparisons). Like in the Iso-Seq data, the 1 aa deletion splice product was not significantly different in PSEN2 in AD versus other samples (FIG. 13A; P=0.059) nor was splicing different for the PSEN1 4 aa deletion product (FIG. 13B; P=0.75), or inclusion of alternative exon 3B (FIG. 13C P=0.70, One-Way ANOVA). It was found that exon 9B inclusion did not correlate with age-at-death (FIG. 14; P (two-tailed) >0.10 for all cohorts).

RNA from the temporal cortex was also sequenced for many of the same samples in the Mayo Clinic dataset (82 AD, 74 controls, 28 pathological aging and 80 PSP). Here, fewer reads were found that mapped to the exon 9B splice junction, with several samples having no reads at all. AD samples averaged 3.09% splicing to exon 9B relative to 2.34% in controls, 1.75% in pathological aging and 2.25% in PSP (FIG. 11C). Levels of this transcript were not significantly different between cohorts (P=0.15, One-Way ANOVA). The number of PSEN2 exon 6 skipping or 77 bp intron inclusion events likewise were minimal in the temporal cortex, and not significantly different between cohorts FIG. 11D; P=0.90, One-Way ANOVA).

An extended PSEN2 3′UTR is subject to RNA editing. During the analysis of PSEN2 N141I transcripts, a series of A-to-G changes present in the 3′UTR sequence were observed. Upon further inspection of these variants, it was found that they were scattered across all reads in a non-allelic manner (i.e., they were not specific to one haplotype). The A-to-G nature suggested instead that these are RNA editing events, reinforced by their enriched abundance at AluJb and AluY short interspersed nuclear elements (SINEs) in the 3′UTR sequence. Adenosine deaminases acting on RNA (ADAR)-mediated editing of adenosine to inosine nucleosides can be detected by the presence of A-to-G mismatches in the cDNA as reverse transcription enzymes incorporate guanidine residues at inosine positions.

Two 3′UTRs for PSEN2 (a short 3′UTR of 507 bp and long 3′UTR of 2976 bp; FIG. 12A) were identified. The long 3′UTR included the Alu elements and is observed a third of the time across samples. The level of RNA editing at Alu elements in the 3′UTR of PSEN2 was comparable in sporadic AD cases, PSEN variant carriers, and controls, when considering uniquely mapped reads (FIG. 12B; p=0.69, One-Way ANOVA). However, the larger dataset from the Mayo Clinic also permitted evaluation of RNA editing at Alu sites in the 3′UTR of PSEN2. Here, it was found that RNA editing was significantly enriched in both AD and PSP samples relative to controls (P<0.0001 for both), and in PSP relative to pathological aging samples (FIG. 12C; P=0.011, One-Way ANOVA followed by Tukey's multiple comparisons). The presence of both individual reads were detected with several edits, and individual residues with several edits (examples shown in FIG. 12D).

Motivated by the identification of RNA editing in the 3′UTR of PSEN2, alternative exons 3B in PSEN1 and 9B in PSEN2 were revisited, both of which include sections of repetitive elements. The PSEN1 exon 3B sequence includes portions of AluJB and L1MB2 long interspersed nuclear element (LINE) repeats. There are several RNA edits in this region, though the level of editing is low across sporadic AD, PSEN variant carriers and controls. The alternative PSEN2 acceptor site in exon 9B is in an L2 LINE. While RNA editing events were not detected, it is possible that some of the adjacent intronic sequence is edited, thus strengthening the splice acceptor sequence.

Discussion In this example, long-read Iso-Seq was used to identify transcript variation in PSEN1 and PSEN2 in cases of sporadic and familial AD. The Iso-Seq findings for PSEN1 match its established gain-of-function role in AD. Equivalent expression of wildtype and variant alleles were identified for four different PSEN1 pathogenic variants that were analyzed. It was observed that a high degree of splicing fidelity—even in aged individuals—with little to no splicing differences between sporadic AD versus controls (FIGS. 5A-5E). In contrast, the findings illuminate a previously under-appreciated role for PSEN2 alternative splicing in AD. Patients with the N141/Volga German pathogenic variant were biased towards skipping of exon 6 and loss of the variant allele, skewing the ratio of mapped reads towards wildtype PSEN2. How the variant might promote exon 6 skipping is currently unknown, though the variant does not overlap an established binding site for HMGA1A, which can promote exon 6 skipping. In addition, increased expression of the wildtype PSEN2 allele was found in a carrier of the K115fs variant, while the PSEN2 isoforms associated with the 2 bp deletion were often linked to a 77 bp intron retention product, leading to an in-frame protein product and confirming previous work (Braggin J E, et al. Ann Clin Transl Neurol. April 2019; 6(4):762-777.).

In addition, significant transcript differences were identified in sporadic AD cases versus controls and pathogenic PSEN variant carriers including more non-productive transcripts (FIGS. 6A-6F). It was found that it was in the sporadic AD cases that demonstrated the greatest abundance of non-productive intron 77 retention and exon 9B products (FIGS. 6A-6F). The results indicate that PSEN1 and PSEN2 are obligated to form full-length transcripts. In addition, these data show that alternative splicing of PSEN2 is not unique to Alzheimer's disease variants, but rather that transcript differences in PSEN2 may play a role in the onset of sporadic AD.

The confirmation that the PSEN2 exon 9B splice product is elevated in sporadic AD cases in an external RNA-seq dataset (FIGS. 11A-11D) indicates that the exon 9B product plays a role in AD etiology. Exon 9B inclusion is highest in the cerebellum and this splice product is also included in PSP cerebellum samples (FIG. 12A). PSP exhibits tau pathology but not Aβ aggregation (Dickson D W, et al. Curr Opin Neurol. August 2010; 23(4):394-400.), which differentiates it from AD pathology.

The discovery of SINE (Alu) and LINE elements in a long 3′UTR of PSEN2 indicates the cell-specific usage of these isoforms. RNA editing of Alu repeat-containing 3′UTRs invokes a proposed mechanism to restrict the transcripts to the nucleus; if they are exported to the cytoplasm, they could precipitate a potent immune response (Hundley H A, Bass B L. Trends Biochem Sci. July 2010; 35(7):377-83.). When the edited mRNA is reported to the cytoplasm, reduced levels of polysomes are found on the edited RNA (Hundley H A, et al. RNA. October 2008; 14(10):2050-60.), indicating that this isoform of PSEN2 may have reduced translation capacity.

Finally, the presence of alternative splice products with non-productive exons deep within traditional introns of both PSEN1 and PSEN2 indicates that intronic variants surrounding these sites promotes inclusion of these non-productive exons and act as risk factors for AD. The increasing number of whole genome sequences available for AD allows for screening for these rare variants and testing for exon inclusion in functional assays, such as minigene reporters.

Using long-read sequencing technology, the understanding of PSEN1 and PSEN2 alternative splicing has been considerably expanded, showing that pathogenic variants in PSEN1 and PSEN2 lead to disease not through haploinsufficiency but rather an alteration of function, and identified novel splice isoforms associated with disease. Specifically, it was found that transcript differences in PSEN2 play an important role in the onset of sporadic AD—the form of which accounts for >95% of AD cases (Masters C L, et al. Nat. Rev. Dis. Primers. Oct. 15 2015; 1:15056). The findings also reveal a direct connection between sporadic and familial AD, indicating that AD is caused by disrupted production or clearance of Aβ aggregates. Thus, based on these results, attenuating the formation of some of the alternative transcripts is a promising targeted therapeutic intervention.

Web Resources. Integrative Genomics Viewer:
https://software.broadinstitute.org/software/igv/

AMP-AD Knowledge Portal (Synapse): https://synapse.org/

Data and Code Availability. RNA sequence reads from the Mayo Clinic are available from synapse.org with accession number syn5550404.

### Example 2

Methods. RNA was extracted from temporal gyrus and parietal lobe tissue samples from individuals with Alzheimer's disease or non-demented controls using the RNeasy Lipid Tissue Mini Kit (Qiagen). RNA was converted to cDNA and then PCR amplified using the following primers, which amplify both wildtype PSEN2 and PSEN2 exon 9B: Forward, 5′-TCACCTATATCTACCTTGGGGAAG (SEQ ID NO: 191) and Reverse: 5′-CCAGCACACTGTAGAAGATGAAGT (SEQ ID NO: 192). PCR products were run on an agarose gel and the bands corresponding to wildtype PSEN2 (570 base pairs) and a PSEN2 exon 9B product (880 base pairs) were quantified using ImageJ software. The percentage of exon 9B product was calculated for each sample and compared using a Student's T-test.

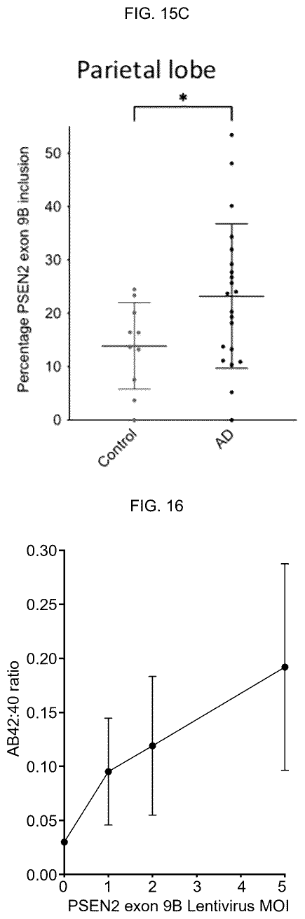

Results. FIGS. 15A-15C illustrate confirmation of PSEN2 exon 9B alternative splicing in parietal lobe and temporal gyrus brain regions in individuals with sporadic Alzheimer's disease (AD) versus control (Ctrl). In both the temporal gyrus and parietal lobe, levels of PSEN2 exon 9B inclusion were higher in Alzheimer's disease cases (FIGS. 15A-15C).

### Example 3

Methods. A flag-tagged version of full-length PSEN2x9B lentivirus was prepared and purified at the Fred Hutchinson viral vector core. Human Amyloid Beta (AB)40 and AB42 were measured by sandwich ELISA kits purchased from Invitrogen (catalog numbers KHB3481 and KHB3544 respectively). Measurements were compared to human AB40 or AB42 synthetic peptide standard curves.

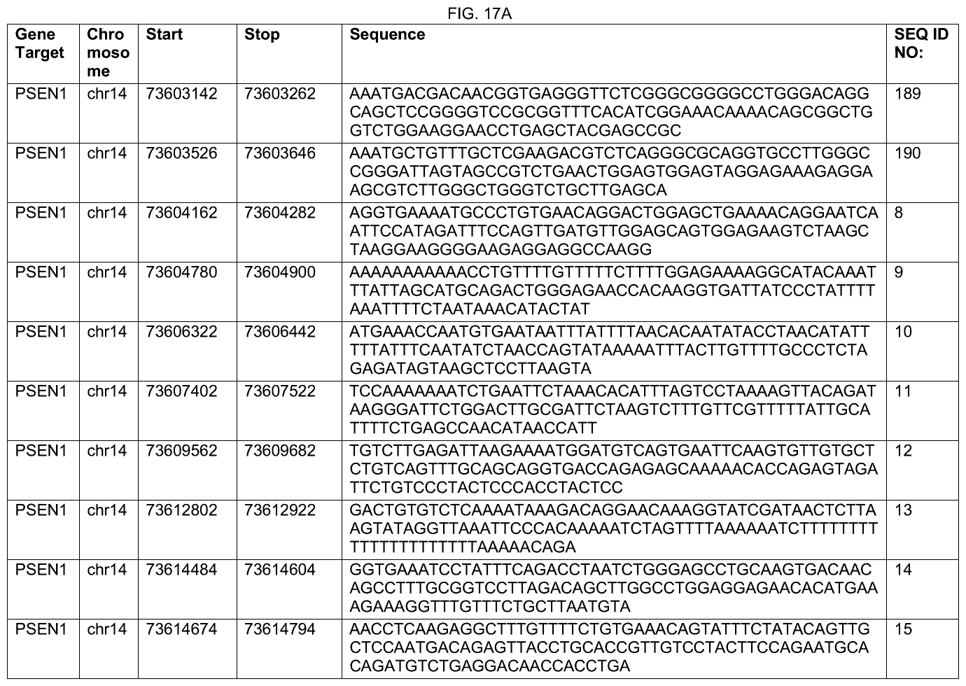

Results. To evaluate the consequences of inclusion of cryptic exon 9B in the PSEN2 transcript, a lentivirus construct expressing the PSEN2 coding sequence with exon 9B included (PSEN2x9B) in the mRNA was designed. Control induced pluripotent stem cell (iPSC) lines were differentiated into neurons (iNeurons). Importantly, these iNeurons were not associated with any AD mutations. PSEN2x9B lentivirus was administered at increasing multiplicity of infection (1×, 2×, and 5×), and cell media was then collected. Successful expression of PSEN2x9B mRNA was confirmed by quantitative RT-PCR. An impaired amyloid beta ratio of AB42 to AB40 is considered a hallmark and biomarker of AD. When measuring human AB40 and AB42 levels by ELISA, an increased ratio of AB42:40 in a dose-dependent manner was found (FIG. 16). At 5×MOI, the AB42:40 ratio of 0.2 is comparable to pathogenic mutations in APP, PSEN1 and PSEN2 from several iPSC-derived neuron studies (Mertens, J., et al. Stem Cell Reports. 2013; 1: 491-498.; Yagi, T., et al. Hum Mol Genet. 2011; 20:4530-4539.; Arber, C., et al. Mol Psychiatry. 2020; 25:2919-2931.).

Closing Paragraphs. The nucleic acid and amino acid sequences provided herein are shown using letter abbreviations for nucleotide bases and amino acid residues, as defined in 37 C.F.R. § 1.822 and set forth in the tables in WIPO Standard ST.25 (1998), Appendix 2, Tables 1 and 3. Only one strand of each nucleic acid sequence is shown, but the complementary strand is understood as included in embodiments where it would be appropriate.

Variants of the sequences disclosed and referenced herein are also included. Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Those of ordinary skill in the art should understand when a sequence should be interpreted as a DNA sequence, as a complementary DNA (cDNA) sequence, or as an RNA sequence. In particular embodiments, a cDNA sequence is reverse transcribed from an RNA sequence. In particular embodiments, a “transcript” refers to an RNA sequence transcribed from a DNA sequence of a DNA sequence transcribed from an RNA sequence. A transcript that includes a cDNA sequence allows inference of the RNA sequence from which it was transcribed. In particular embodiments, an RNA sequence is reverse transcribed into cDNA for sequencing. In particular embodiments, a transcript sequence (e.g., the RNA sequence) may be detected in a sample by detecting the reverse-transcribed cDNA sequence.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. In particular embodiments, a material effect would cause a statistically significant decrease in an ability to treat or diagnose a tauopathy according to a method described herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

1-70. (canceled)

71. A composition comprising a reagent that targets a pathogenic transcript variant of PSEN2, thereby inhibiting gene expression of the pathogenic transcript variant of PSEN2.

72. The composition of claim 71, wherein the reagent has sufficient complementarity to the pathogenic transcript variant of PSEN2; or
wherein the reagent comprises at least one of an inhibitory nucleic acid molecule, an antisense oligonucleotide, a short hairpin RNA (shRNA), a small interfering RNA (siRNA), or a microRNA (miRNA).

73. The composition of claim 72, wherein the inhibitory nucleic acid molecule hybridizes to a portion of a sequence set forth in SEQ ID NO: 2 or SEQ ID NO: 6 or a sequence having 90% sequence identity to the sequence set forth in SEQ ID NO: 2 or SEQ ID NO: 6;
wherein the inhibitory nucleic acid molecule has at least 95% reverse complementarity to the sequence set forth in SEQ ID NO: 2 or SEQ ID NO: 6;
wherein the inhibitory nucleic acid molecule is the reverse complement of the sequence set forth in SEQ ID NO: 2 or SEQ ID NO: 6; or
wherein the reagent comprises a viral vector encoding a nucleic acid having sufficient sequence complementarity to the pathogenic transcript variant of PSEN2.

74. A method of treating a neurodegenerative disease comprising administering a therapeutically effective amount of the composition of claim 73 to a subject in need thereof, thereby treating the subject in need thereof.

75. The method of claim 74, wherein the neurodegenerative disease is a tauopathy, Alzheimer's disease, sporadic Alzheimer's disease, familial Alzheimer's disease, or progressive supranuclear palsy.

76. The method of claim 74, wherein the reagent comprises:
a nucleic acid having sufficient sequence complementarity to the pathogenic transcript variant of PSEN2;
an inhibitory nucleic acid molecule;
an antisense oligonucleotide, a short hairpin RNA (shRNA), a small interfering RNA (siRNA), and/or a microRNA (miRNA); or
splice-switching oligonucleotides.

77. The method of claim 76, wherein the pathogenic transcript variant of PSEN2 comprises at least one of:
a transcript with the inclusion of exon 9B;
a transcript with the inclusion of 77 bp of the intronic sequence prior to exon 6;
the sequence set forth in SEQ ID NO: 2, SEQ ID NO: 3, SEQ ID NO: 4, SEQ ID NO: 5, SEQ ID NO: 6, or SEQ ID NO: 7; or
a sequence having 90% sequence identity to the sequence set forth in SEQ ID NO: 2, SEQ ID NO: 3, SEQ ID NO: 4, SEQ ID NO: 5, SEQ ID NO: 6, or SEQ ID NO: 7.

78. The method of claim 77, wherein the reagent hybridizes to a pathogenic transcript variant of PSEN2 comprising the sequence set forth in SEQ ID NO: 3, SEQ ID NO: 4, SEQ ID NO: 5, or SEQ ID NO: 7 or a sequence with at least 90% identity to the sequence set forth in SEQ ID NO: 3, SEQ ID NO: 4, SEQ ID NO: 5, or SEQ ID NO: 7;
wherein the reagent has at least 95% reverse complementarity to the sequence set forth in SEQ ID NO: 3, SEQ ID NO: 4, SEQ ID NO: 5, or SEQ ID NO: 7; or
wherein the reagent is the reverse complement of the sequence set forth in SEQ ID NO: 3, SEQ ID NO: 4, SEQ ID NO: 5, or SEQ ID NO: 7.

79. The method of claim 76, wherein the pathogenic transcript variant of PSEN2 is not wildtype PSEN2 transcript, the wildtype PSEN2 transcript having the sequence set forth in SEQ ID NO: 1.

80. The method of claim 76, wherein the inhibitory nucleic acid molecule has at least 95% reverse complementarity to the sequence set forth in SEQ ID NO: 2 or SEQ ID NO: 6;
wherein the inhibitory nucleic acid molecule is the reverse complement of the sequence set forth in SEQ ID NO: 2 or SEQ ID NO: 6; or
wherein the inhibitory nucleic acid molecule hybridizes to a portion of a sequence set forth in SEQ ID NO: 2, SEQ ID NO: 3, SEQ ID NO: 4, SEQ ID NO: 5, SEQ ID NO: 6, or SEQ ID NO: 7 or a sequence having 90% sequence identity to the sequence set forth in SEQ ID NO: 2, SEQ ID NO: 3, SEQ ID NO: 4, SEQ ID NO: 5, SEQ ID NO: 6, or SEQ ID NO: 7.

81. The method of claim 76, wherein the splice-switching oligonucleotides have at least 95% reverse complementarity to the sequence set forth in SEQ ID NO: 2 or SEQ ID NO: 6;
wherein the splice-switching oligonucleotides are the reverse complement of the sequence set forth in SEQ ID NO: 2 or SEQ ID NO: 6; or
wherein splice-switching oligonucleotides hybridize to a portion of a sequence set forth in SEQ ID NO: 2, SEQ ID NO: 3, SEQ ID NO: 4, SEQ ID NO: 5, SEQ ID NO: 6, or SEQ ID NO: 7 or a sequence having 90% sequence identity to the sequence set forth in SEQ ID NO:
2, SEQ ID NO: 3, SEQ ID NO: 4, SEQ ID NO: 5, SEQ ID NO: 6, or SEQ ID NO: 7.

82. The method of claim 74, wherein the reagent comprises a viral vector encoding a nucleic acid having sufficient sequence complementarity to the pathogenic transcript variant of PSEN2.

83. A polynucleotide complementary to a pathogenic transcript variant of PSEN2 conjugated to a tag.

84. The polynucleotide of claim 83, wherein the polynucleotide comprises the sequence set forth in SEQ ID NO: SEQ ID NO: 2, SEQ ID NO: 3, SEQ ID NO: 4, SEQ ID NO: 5, SEQ ID NO: 6, or SEQ ID NO: 7 or a sequence having 90% sequence identity to the sequence set forth in SEQ ID NO: 2, SEQ ID NO: 3, SEQ ID NO: 4, SEQ ID NO: 5, SEQ ID NO: 6, or SEQ ID NO: 7 and/or
wherein the tag comprises a fluorescent probe, an affinity tag, a barcode, an adapter, a hybridization probe, a molecular beacon, or a chromogenic probe.

85. A method of diagnosing a tauopathy comprising:
obtaining a tissue sample derived from a subject;
detecting a level of pathogenic transcript variants of PSEN2 in the tissue sample; and
based on detecting the level of pathogenic transcript variants of PSEN2, diagnosing the subject as having the tauopathy.

86. The method of claim 85, wherein the level of pathogenic transcript variants of PSEN2 comprises a ratio of pathogenic transcript variants of PSEN2 per sample to wild-type transcripts of PSEN2 per sample.

87. The method of claim 85, wherein the pathogenic transcript variants of PSEN2 comprise at least one of:
a transcript with the inclusion of exon 9B;
a transcript with the inclusion of 77 bp of the intronic sequence prior to exon 6;
the sequence set forth in SEQ ID NO: 2, SEQ ID NO: 3, SEQ ID NO: 4, SEQ ID NO: 5, SEQ ID NO: 6, and/or SEQ ID NO: 7; or
a sequence having 90% sequence identity to the sequence set forth in SEQ ID NO: 2, SEQ ID NO: 3, SEQ ID NO: 4, SEQ ID NO: 5, SEQ ID NO: 6, and/or SEQ ID NO: 7.

88. The method of claim 85, wherein the detecting comprises extracting RNA from the tissue sample; converting the RNA into cDNA; amplifying cDNA; and performing sequencing analysis; or
wherein the detecting comprises quantifying PSEN2 reads per sample; quantifying select alternative splicing isoforms of PSEN2 reads per sample; and
determining a percentage of select alternative splicing isoforms of PSEN2 compared to PSEN2 reads per sample.

89. The method of claim 85, wherein diagnosing the subject comprises determining that the level of pathogenic transcript variants of PSEN2 is higher than a reference level, the reference level being the level of pathogenic transcript variants of PSEN2 in subjects without a neuropathologic disease.

90. The method of claim 85, wherein the tauopathy is sporadic Alzheimer's disease.


SEQUENCE LISTING (192 sequences)

SEQ ID NO: 1 - DNA, 1344 bp, Homo sapiens
  source 1..1344: mol_type = other DNA
atgctcacattcatggcctctgacagcgaggaagaagtgtgtgatgagcggacgtccctaatgtcggctgagagccccacgccgcgctcctgccaggagggcaggcagggcccagaggatggagagaacactgcccagtggagaagccaggagaacgaggaggacggtgaggaggaccctgaccgctatgtctgtagtggggttcccgggcggccgccaggcctggaggaagagctgaccctcaaatacggagcgaagcacgtgatcatgctgtttgtgcctgtcactctgtgcatgatcgtggtggtagccaccatcaagtctgtgcgcttctacacagagaagaatggacagctcatctacacgccattcactgaggacacaccctcggtgggccagcgcctcctcaactccgtgctgaacaccctcatcatgatcagcgtcatcgtggttatgaccatcttcttggtggtgctctacaagtaccgctgctacaagttcatccatggctggttgatcatgtcttcactgatgctgctgttcctcttcacctatatctaccttggggaagtgctcaagacctacaatgtggccatggactaccccaccctcttgctgactgtctggaacttcggggcagtgggcatggtgtgcatccactggaagggccctctggtgctgcagcaggcctacctcatcatgatcagtgcgctcatggccctagtgttcatcaagtacctcccagagtggtccgcgtgggtcatcctgggcgccatctctgtgtatgatctcgtggctgtgctgtgtcccaaagggcctctgagaatgctggtagaaactgcccaggagagaaatgagcccatattccctgccctgatatactcatctgccatggtgtggacggttggcatggcgaagctggacccctcctctcagggtgccctccagctcccctacgacccggagatggaagactcctatgacagttttggggagccttcataccccgaagtctttgagcctcccttgactggctacccaggggaggagctggaggaagaggaggaaaggggcgtgaagcttggcctcggggacttcatcttctacagtgtgctggtgggcaaggcggctgccacgggcagcggggactggaataccacgctggcctgcttcgtggccatcctcattggcttgtgtctgaccctcctgctgcttgctgtgttcaagaaggcgctgcccgccctccccatctccatcacgttcgggctcatcttttacttctccacggacaacctggtgcggccgttcatggacaccctggcctcccatcagctctacatctga

SEQ ID NO: 2 - DNA, 310 bp, Homo sapiens
  source 1..310: mol_type = other DNA
gcactgtgctaggcattgggggacccagcaggaaagaagaccacagggtcccaggcctcatggagctcacggccctgtgattgtgatgccctcggtctgttgatggcggggcttaaatagcctgaatttctggagctctggcgtctgcaaggtggcctgggaaagagtttatggaacagctacagagttctaggtaccttcatgcagttgaggattcgagcccgtagaggagaatcgcctgcagcgtggccccacgggaaagcacattccaggcgcattccgaggatgagcggagaccatgtatggaaag

SEQ ID NO: 3 - DNA, 1662 bp, Homo sapiens
  source 1..1662: mol_type = other DNA
atgctcacattcatggcctctgacagcgaggaagaagtgtgtgatgagcggacgtccctaatgtcggctgagagccccacgccgcgctcctgccaggagggcaggcagggcccagaggatggagagaacactgcccagtggagaagccaggagaacgaggaggacggtgaggaggaccctgaccgctatgtctgtagtggggttcccgggcggccgccaggcctggaggaagagctgaccctcaaatacggagcgaagcacgtgatcatgctgtttgtgcctgtcactctgtgcatgatcgtggtggtagccaccatcaagtctgtgcgcttctacacagagaagaatggacagctcatctacacgccattcactgaggacacaccctcggtgggccagcgcctcctcaactccgtgctgaacaccctcatcatgatcagcgtcatcgtggttatgaccatcttcttggtggtgctctacaagtaccgctgctacaagttcatccatggctggttgatcatgtcttcactgatgctgctgttcctcttcacctatatctaccttggggaagtgctcaagacctacaatgtggccatggactaccccaccctcttgctgactgtctggaacttcggggcagtgggcatggtgtgcatccactggaagggccctctggtgctgcagcaggcctacctcatcatgatcagtgcgctcatggccctagtgttcatcaagtacctcccagagtggtccgcgtgggtcatcctgggcgccatctctgtgtatgatctcgtggctgtgctgtgtcccaaagggcctctgagaatgctggtagaaactgcccaggagagaaatgagcccatattccctgccctgatatactcatgcactgtgctaggcattgggggacccagcaggaaagaagaccacagggtcccaggcctcatggagctcacggccctgtgattgtgatgccctcggtctgttgatggcggggcttaaatagcctgaatttctggagctctggcgtctgcaaggtggcctgggaaagagtttatggaacagctacagagttctaggtaccttcatgcagttgaggattcgagcccgtagaggagaatcgcctgcagcgtggccccacgggaaagcacattccaggcgcattccgaggatgagcggagaccatgtatggaaagctgccatggtgtggacggttggcatggcgaagctggacccctcctctcagggtgccctccagctcccctacgacccggagatggaagaagactcctatgacagttttggggagccttcataccccgaagtctttgagcctcccttgactggctacccaggggaggagctggaggaagaggaggaaagtaagggggcgtgaagcttggcctcggggacttcatcttctacagtgtgctggtgggcaaggcggctgccacgggcagcggggactggaataccacgctggcctgcttcgtggccatcctcattggcttgtgtctgaccctcctgctgcttgctgtgttcaagaaggcgctgcccgccctccccatctccatcacgttcgggctcatcttttacttctccacggacaacctggtgcggccgttcatggacaccctggcctcccatcagctctacatctga

SEQ ID NO: 4 - DNA, 1870 bp, Homo sapiens
  source 1..1870: mol_type = other DNA
atgctcacattcatggcctctgacagcgaggaagaagtgtgtgatgagcggacgtccctaatgtcggccgagagccccacgccgcgctcctgccaggagggcaggcagggcccagaggatggagagaatactgcccagtggagaagccaggagaacgaggaggacggtgaggaggaccctgaccgctatgtctgtagtggggttcccgggcggccgccaggcctggaggaagagctgaccctcaaatacggagcgaagcatgtgatcatgctgtttgtgcctgtcactctgtgcatgatcgtggtggtagccaccatcaagtctgtgcgcttctacacagagaagaatggacagctcatctacacgccattcactgaggacacaccctcggtgggccagcgcctcctcaactccgtgctgaacaccctcatcatgatcagcgtcatcgtggttatgaccatcttcttggtggtgctctacaagtaccgctgctacaagttcatccatggctggttgatcatgtcttcactgatgctgctgttcctcttcacctatatctaccttggggaagtgctcaagacctacaatgtggccatggactaccccaccctcttgctgactgtctggaacttcggggcagtgggcatggtgtgcatccactggaagggccctctggtgctgcagcaggcctacctcatcatgatcagtgcgctcatggccctagtgttcatcaagtacctcccagagtggtccgcgtgggtcatcctgggcgccatctctgtgtatgatctcgtggctgtgctgtgtcccaaagggcctctgagaatgctggtagaaactgcccaggagagaaatgagcccatattccctgccctgatatactcatgcactgtgctaggcattgggggacccagcaggaaagaagaccacagggtcccaggcctcatggagctcacggccctgtgattgtgatgccctcggtctgttgatggcggggcttaaatagcctgaatttctggagctctggcgtctgcaaggtggcctgggaaagagtttatggaacagctacagagttctaggtaccttcatgcagttgaggattcgagcccgtagaggagaattgcctgcagcgtggccccacgggaaagcacattccaggcgcattccgaggatgagcggagaccatgtatggaaaggaggggagacaaacagtaacagaatagacaaatgcaagagagagtgactctggacccctcccacaacggcctcctaacaatggagcatgagcagatacctgcaggatggagggtcctgtgcaggctttctgggacgcagactggccacctcccccaggccctgcaggcagccactgttagcaccgcctgagacgtgaaccttttctcctcccccagctgccatggtgtggacggttggcatggcgaagctggacccctcctctcagggtgccctccagctcccctacgacccggagatggaagactcctatgacagttttggggagccttcataccccgaagtctttgagcctcccttgactggctacccaggggaggagctggaggaagaggaggaaaggggcgtgaagcttggcctcggggacttcatcttctacagtgtgctggtgggcaaggcggctgccacgggcagcggggactggaataccacgctggcctgcttcgtggccatcctcattggcttgtgtctgaccctcctgctgcttgctgtgttcaagaaggcgctgcccgccctccccatctccatcacgttcgggctcatcttttacttctccacggacaacctggtgcggccgttcatggacaccctggcctcccatcagctctacatctga

SEQ ID NO: 5 - DNA, 1826 bp, Homo sapiens
  source 1..1826: mol_type = other DNA
atgctcacattcatggcctctgacagcgaggaagaagtgtgtgatgagcggacgtccctaatgtcggccgagagccccacgccgcgctcctgccaggagggcaggcagggcccagaggatggagagaatactgcccagtggagaagccaggagaacgaggaggacggtgaggaggaccctgaccgctatgtctgtagtggggttcccgggcggccgccaggcctggaggaagagctgaccctcaaatacggagcgaagcatgtgatcatgctgtttgtgcctgtcactctgtgcatgatcgtggtggtagccaccatcaagtctgtgcgcttctacacagagaagaatggacagctcatctacacgccattcactgaggacacaccctcggtgggccagcgcctcctcaactccgtgctgaacaccctcatcatgatcagcgtcatcgtggttatgaccatcttcttggtggtgctctacaagtaccgctgctacaagttcatccatggctggttgatcatgtcttcactgatgctgctgttcctcttcacctatatctaccttggggaagtgctcaagacctacaatgtggccatggactaccccaccctcttgctgactgtctggaacttcggggcagtgggcatggtgtgcatccactggaagggccctctggtgctgcagcaggcctacctcatcatgatcagtgcgctcatggccctagtgttcatcaagtacctcccagagtggtccgcgtgggtcatcctgggcgccatctctgtgtatggttaaagaattggaggctcagagaagtgggactccccagcctggccaccgcgtctcgggtgcacagctcctccatgcttgcagttgcctgcgaggccctactctggctcacaccagggcctgctctaagttgtgactggagaatgagaatttgggatgccagcccagagatctcgtggctgtgctgtgtcccaaagggcctctgagaatgctggtagaaactgcccaggagagaaatgagcccatattccctgccctgatatactcatgcactgtgctaggcattgggggacccagcaggaaagaagaccacagggtcccaggcctcatggagctcacggccctgtgattgtgatgccctcggtctgttgatggcggggcttaaatagcctgaatttctggagctctggcgtctgcaaggtggcctgggaaagagtttatggaacagctacagagttctaggtaccttcatgcagttgaggattcgagcccgtagaggagaattgcctgcagcgtggccccacgggaaagcacattccaggcgcattccgaggatgagcggagaccatgtatggaaagctgccatggtgtggacggttggcatggcgaagctggacccctcctctcagggtgccctccagctcccctacgacccggagatggaagaagactcctatgacagttttggggagctttcataccccgaagtctttgagcctcccttgactggctacccaggggaggagctggaggaagaggaggaaaggggcgtgaagcttggcctcggggacttcatcttctacagtgtgctggtgggcaaggcggctgccacgggcagcggggactggaataccacgctggcctgcttcgtggccatcctcattggcttgtgtctgaccctcctgctgcttgctgtgttcaagaaggcgctgcccgccctccccatctccatcacgttcgggctcatcttttacttctccacggacaacctggtgcggccgttcatggacaccctggcctcccatcagctctacatctga

SEQ ID NO: 6 - DNA, 77 bp, Homo sapiens
  source 1..77: mol_type = other DNA
gtccagaatcactcaaggtggggagcctcgaggagcagtcagggccgggagcatcagccctttgccttctccctcag

SEQ ID NO: 7 - DNA, 1424 bp, Homo sapiens
  source 1..1424: mol_type = other DNA
atgctcacattcatggcctctgacagcgaggaagaagtgtgtgatgagcggacgtccctaatgtcggctgagagccccacgccgcgctcctgccaggagggcaggcagggcccagaggatggagagaacactgcccagtggagaagccaggagaacgaggaggacggtgaggaggaccctgaccgctatgtctgtagtggggttcccgggcggccgccaggcctggaggaagagctgaccctcaaatacggagcgaagcacgtgatcatgctgtttgtgcctgtcactctgtgcatgatcgtggtggtagccaccatcaagtctgtgcgcttctacacagagaagaatggacagctgtccagaatcactcaaggtggggagcctcgaggagcagtcagggccgggagcatcagccctttgccttctccctcagcatctacacgccattcactgaggacacaccctcggtgggccagcgcctcctcaactccgtgctgaacaccctcatcatgatcagcgtcatcgtggttatgaccatcttcttggtggtgctctacaagtaccgctgctacaagttcatccatggctggttgatcatgtcttcactgatgctgctgttcctcttcacctatatctaccttggggaagtgctcaagacctacaatgtggccatggactaccccaccctcttgctgactgtctggaacttcggggcagtgggcatggtgtgcatccactggaagggccctctggtgctgcagcaggcctacctcatcatgatcagtgcgctcatggccctagtgttcatcaagtacctcccagagtggtccgcgtgggtcatcctgggcgccatctctgtgtatgatctcgtggctgtgctgtgtcccaaagggcctctgagaatgctggtagaaactgcccaggagagaaatgagcccatattccctgccctgatatactcatctgccatggtgtggacggttggcatggcgaagctggacccctcctctcagggtgccctccagctcccctacgacccggagatggaagaagactcctatgacagttttggggagccttcataccccgaagtctttgagcctcccttgactggctacccaggggaggagctggaggaagaggaggaaaggggcgtgaagcttggcctcggggacttcatcttctacagtgtgctggtgggcaaggcggctgccacgggcagcggggactggaataccacgctggcctgcttcgtggccatcctcattggcttgtgtctgaccctcctgctgcttgctgtgttcaagaaggcgctgcccgccctccccatctccatcacgttcgggctcatcttttacttctccacggacaacctggtgcggccgttcatggacaccctggcctcccatcagctctacatctga

SEQ ID NO: 8 - DNA, 120 bp, Homo sapiens
  source 1..120: mol_type = genomic DNA
aggtgaaaatgccctgtgaacaggactggagctgaaaacaggaatcaattccatagatttccagttgatgttggagcagtggagaagtctaagctaaggaaggggaagaggaggccaagg

SEQ ID NO: 9 - DNA, 120 bp, Homo sapiens
  source 1..120: mol_type = genomic DNA
aaaaaaaaaaacctgttttgtttttcttttggagaaaaggcatacaaatttattagcatgcagactgggagaaccacaaggtgattatccctattttaaattttctaataaacatactat

SEQ ID NO: 10 - DNA, 120 bp, Homo sapiens
  source 1..120: mol_type = genomic DNA
atgaaaccaatgtgaataatttattttaacacaatatacctaacatatttttatttcaatatctaaccagtataaaaatttacttgttttgccctctagagatagtaagctccttaagta

SEQ ID NO: 11 - DNA, 120 bp, Homo sapiens
  source 1..120: mol_type = genomic DNA
tccaaaaaaatctgaattctaaacacatttagtcctaaaagttacagataagggattctggacttgcgattctaagtctttgttcgtttttattgcattttctgagccaacataaccatt

SEQ ID NO: 12 - DNA, 120 bp, Homo sapiens
  source 1..120: mol_type = genomic DNA
tgtcttgagattaagaaaatggatgtcagtgaattcaagtgttgtgctctgtcagtttgcagcaggtgaccagagagcaaaaacaccagagtagattctgtccctactcccacctactcc

SEQ ID NO: 13 - DNA, 120 bp, Homo sapiens
  source 1..120: mol_type = genomic DNA
gactgtgtctcaaaataaagacaggaacaaaggtatcgataactcttaagtataggttaaattcccacaaaaatctagttttaaaaaatctttttttttttttttttttttaaaaacaga

SEQ ID NO: 14 - DNA, 120 bp, Homo sapiens
  source 1..120: mol_type = genomic DNA
ggtgaaatcctatttcagacctaatctgggagcctgcaagtgacaacagcctttgcggtccttagacagcttggcctggaggagaacacatgaaagaaaggtttgtttctgcttaatgta

SEQ ID NO: 15 - DNA, 120 bp, Homo sapiens
  source 1..120: mol_type = genomic DNA
aacctcaagaggctttgttttctgtgaaacagtatttctatacagttgctccaatgacagagttacctgcaccgttgtcctacttccagaatgcacagatgtctgaggacaaccacctga

SEQ ID NO: 16 - DNA, 120 bp, Homo sapiens
  source 1..120: mol_type = genomic DNA
aggacacagggctgtttaacacataggtgttgatacagttcctacctaattcacatatcaccacctcagttgaagccttctccagcttcccaaggtgtgcagcgcctcatagttcatgtt

SEQ ID NO: 17 - DNA, 120 bp, Homo sapiens
  source 1..120: mol_type = genomic DNA
aattttcccacagttatggtacatctgggctgccttgtaggcttgtgtgtacatttcactacaaacagtcacaaaaggctgaaaggccactccagcaagatctgtagaggattagtccgt

SEQ ID NO: 18 - DNA, 120 bp, Homo sapiens
  source 1..120: mol_type = genomic DNA
actataatatttatgtacataaatcgtatcgcattatgtaatacacttggcatgttttaaaactttaggctgggtgtggtggctcacgcctgtaatcccagcaatttgggaggccgaggc

SEQ ID NO: 19 - DNA, 120 bp, Homo sapiens
  source 1..120: mol_type = genomic DNA
ggtgatcttggtgatttccccctttcattttctaaatgttctataatacagttatttaataatttaaaaagctattttgaaaaagtaatctcacgctgtcaccaaagctggagtgcagtg

SEQ ID NO: 20 - DNA, 120 bp, Homo sapiens
  source 1..120: mol_type = genomic DNA
ccagtgcagtgtgacagagtgaggctgtctcaaaaaaagaaaaaaaaaacccacaaaagctcggtagcatgagatctttgagagcattcttatgttcttattggtaattggagtttatat

SEQ ID NO: 21 - DNA, 120 bp, Homo sapiens
  source 1..120: mol_type = genomic DNA
ttaagtcattttttttttttttgcccattaagtcttaaaggattaatacattttctttccttttttttttttttttgaaacggagcctccctctgtcgcccaggctggagttcagttgtg

SEQ ID NO: 22 - DNA, 120 bp, Homo sapiens
  source 1..120: mol_type = genomic DNA
acttaactcagtagcaaaggttacaagcaatatttatggagtaagaaatagaaatggcaataaatatatgaaaagcctagttgatcagccttactaatagggataaaaattaaaacttca

SEQ ID NO: 23 - DNA, 120 bp, Homo sapiens
  source 1..120: mol_type = genomic DNA
tgtatgatactgtaatcatggaaacctgtcattatctgtttatacaaacccatagactgtataacaccaaaaagtgaaccctaaggtaaactgtggactgtcaatgtaggttcatctgtt

SEQ ID NO: 24 - DNA, 120 bp, Homo sapiens
  source 1..120: mol_type = genomic DNA
gtctctgtctaaaaaaaaaacaaaacatacagactcttgtctggcttctttctttcagcataattattttgaaattcgtccatgttatgtggttcctttttattgctgagttgtatacca

SEQ ID NO: 25 - DNA, 120 bp, Homo sapiens
  source 1..120: mol_type = genomic DNA
ttaatggattttaagaattatttacatagtctggatacaagtcctctgtaatatatataatttgcaagtgttttctcccagactgtggcttttctttttattaacagtgctttttgaagg

SEQ ID NO: 26 - DNA, 120 bp, Homo sapiens
  source 1..120: mol_type = genomic DNA
tcatgtaagtggaatcatacattatttctcttttagtgacttgcttattttgcttagcataatgtcctcaaggttcatctatattgtagcatgtgacaagatttcctttttaagggctga

SEQ ID NO: 27 - DNA, 120 bp, Homo sapiens
  source 1..120: mol_type = genomic DNA
tccacctgccttagcctcccaaactgctggggttgtagatgtgagctaccacacccagcctgatttgcagatattttctccagttctgtaggttgccttttcactctgatgactgcttcc

SEQ ID NO: 28 - DNA, 120 bp, Homo sapiens
  source 1..120: mol_type = genomic DNA
gattggtaaaaatctaagcacttgctaacataaggtgttagcaacagtgtggtaaagtaggcaatctcttgcattgctgattctctaaattggtgcaatccacagggagggcaatttggc

SEQ ID NO: 29 - DNA, 120 bp, Homo sapiens
  source 1..120: mol_type = genomic DNA
caagaccagcttgggcaacatggtgaaactttgtctctacaaaaaattagccgagcttcacaaacaatcttacaggagtcattcattaattaactcaacaaatatttatagagtacagga

SEQ ID NO: 30 - DNA, 120 bp, Homo sapiens
  source 1..120: mol_type = genomic DNA
atggtaaaactactgaatgcttaatatgtgccagacaccgtgagatacgtctttgaagtactatcccagtagaatttacaacaaccctatcaggagatactttttttatcaccattttac

SEQ ID NO: 31 - DNA, 120 bp, Homo sapiens
  source 1..120: mol_type = genomic DNA
ttagtcacctccttctgtggaagaatagaagatagacagctccttgctgtctatcacagtatgttctggtcccgttggcctcaccttccatataatcatttccattctactgggctttag

SEQ ID NO: 32 - DNA, 120 bp, Homo sapiens
  source 1..120: mol_type = genomic DNA
atgataggtatgctcaagtctctaacagctctaaaataacgttttctgtttttttttttgtcttcatttctggtggggttggataagaaaagtttttcaagttaaatcactctctattgc

SEQ ID NO: 33 - DNA, 120 bp, Homo sapiens
  source 1..120: mol_type = genomic DNA
aggagagggcaggggctgccaccctgaccctccactgagctggctggcacctggccatccccgaatggccgcgttgaaggagcattgattgtaacacacttggatgctgctgcggggccg

SEQ ID NO: 34 - DNA, 120 bp, Homo sapiens
  source 1..120: mol_type = genomic DNA
aatgacaatagagaacggcaggagcacaacgacagacggagccttggccaccctgagccattatctaatggacgaccccagggtaactcccggcaggtggtggagcaagatgaggaagaa

SEQ ID NO: 35 - DNA, 120 bp, Homo sapiens
  source 1..120: mol_type = genomic DNA
ctttattgcaaggcctgtgagggtactgagtctccagggtgaggtgtataataaatggccaaacgaattgttaattgaaaggatgaacaactaattcaagccagcactctctagactcct

SEQ ID NO: 36 - DNA, 120 bp, Homo sapiens
  source 1..120: mol_type = genomic DNA
gggaagatggatatgtgacattatcttaagacaactccagttgcaattactctgcagatgagaggcactaattataagccatattacctttcttctgacaaccacttgtcagcccacgtg

SEQ ID NO: 37 - DNA, 120 bp, Homo sapiens
  source 1..120: mol_type = genomic DNA
aatctataccccattcacagaagataccgagactgtgggccagagagccctgcactcaattctgaatgctgccatcatgatcagtgtcattgttgtcatgactatcctcctggtggttct

SEQ ID NO: 38 - DNA, 120 bp, Homo sapiens
  source 1..120: mol_type = genomic DNA
tgtaactgaaaaataaacatttccatataatagcacaatctaagtgggtttttgtttgtttgtttgtttgttgaagcagggccttgccctgtcacccaggctggagtgaagtgcagtggc

SEQ ID NO: 39 - DNA, 120 bp, Homo sapiens
  source 1..120: mol_type = genomic DNA
ttccctgctttagtaggctgaccttgtgacttttaccaggatcccattatacttttgttcttctgtttatttctcagattttattggagggtggaattctactttttctttcttataatc

SEQ ID NO: 40 - DNA, 120 bp, Homo sapiens
  source 1..120: mol_type = genomic DNA
ttgttttgagataaagtcttgctttgtcacctaggcaagagtgctaggattacaggcgtgagccgctatgccaggacagccgttttcttctagtattatttgccctctgtctgaacatct

SEQ ID NO: 41 - DNA, 120 bp, Homo sapiens
  source 1..120: mol_type = genomic DNA
ctcttctgggagtgacaacacaaataactttttattccacaggtttctgaggctctgtttattttccaatttttttcactattgttctggttagataatttctattgatctgtcttcaag

SEQ ID NO: 42 - DNA, 120 bp, Homo sapiens
  source 1..120: mol_type = genomic DNA
ccactgcactccagcctgagctacagtgcgagactctgtctccaaaaaaaaggaagaaaaactggatttttacactgtctgggtgttaggagacctctttcctgatcctcaagccagaac

SEQ ID NO: 43 - DNA, 120 bp, Homo sapiens
  source 1..120: mol_type = genomic DNA
atttctggccttgatagtttagggtttagagaagatactgttgtgtactataaaacagcagggctgttttctttcataccctttagcacaggaaagctgaactagggatttttgcaagat

SEQ ID NO: 44 - DNA, 120 bp, Homo sapiens
  source 1..120: mol_type = genomic DNA
cagatagaaaacatgtctgaaagggaaacggttttattttgtttcgtatttttttttgagacaatgtctcactttgtcagtcaggctggaatgtagtggcactgtctcggctcactgcac

SEQ ID NO: 45 - DNA, 120 bp, Homo sapiens
  source 1..120: mol_type = genomic DNA
ggcgacaaagtgaggcttcatctcccaaaaataaataaataaaattaaaaaaagaaataaataagaacatctccttcttcctttgccaaaaaaccgaggtgaaaagcatctctcaagaat

SEQ ID NO: 46 - DNA, 120 bp, Homo sapiens
  source 1..120: mol_type = genomic DNA
aaacaactttgttgtatctggatataatttagaatattcaaatgttggctaggtgcggtggcaacctgtaatcctagcactttgggaggccaaggcaggtggatcacctcaggtcgggag

SEQ ID NO: 47 - DNA, 120 bp, Homo sapiens
  source 1..120: mol_type = genomic DNA
ttatattgaaatgctttcttttctaggtcatccatgcctggcttattatatcatctctattgttgctgttctttttttcattcatttacttggggtaagttgtgaaatttttggtctgtc

SEQ ID NO: 48 - DNA, 120 bp, Homo sapiens
  source 1..120: mol_type = genomic DNA
ttaagactacagttaattctaagcctttttgaagattatatagtcttctagtagaagtctgtgcatcagaatgttttaagaaagggttttaagaatgtataaaaaccaaaataattgatt

SEQ ID NO: 49 - DNA, 120 bp, Homo sapiens
  source 1..120: mol_type = genomic DNA
accgcaccctgccataataatacatttgaccaagtggagtttgtttgcagaatcgttcattataagttgccatattactagatcatcatctctgtatgtgctaaaaggcatttgataaga

SEQ ID NO: 50 - DNA, 120 bp, Homo sapiens
  source 1..120: mol_type = genomic DNA
gctgcttgggaggctgagtcaggaggatcacttgagccaaggactttggggctgcagtgaactgtggttgtgccctctatccagggtgacagtcagaccttgtctctatttaaaaaaaaa

SEQ ID NO: 51 - DNA, 120 bp, Homo sapiens
  source 1..120: mol_type = genomic DNA
ctttatttaagcataaactttgggtttcagatactgtctggaaaaatgatttatctcccactttgaaattccaaaatacgtacatatattttttttttcttttcttttttagttttaggg

SEQ ID NO: 52 - DNA, 120 bp, Homo sapiens
  source 1..120: mol_type = genomic DNA
ggaagtgtttaaaacctataacgttgctgtggactacattactgttgcactcctgatctggaattttggtgtggtgggaatgatttccattcactggaaaggtccacttcgactccagca

SEQ ID NO: 53 - DNA, 120 bp, Homo sapiens
  source 1..120: mol_type = genomic DNA
tcattttttaaacgatttttttcctttatatcttactgcagaagcttttttcttttttttgagacaaagtcttgctgtgtcacccaggctggggtgcagtggcacagtcatagctcactg

SEQ ID NO: 54 - DNA, 120 bp, Homo sapiens
  source 1..120: mol_type = genomic DNA
caaagtgctgagattacaggtgtgagccactatacccagcccagtgttatatttttgtataatcctatgaagtatcaaggcagttattatccctgttttactgctaagaaacttgaagtt

SEQ ID NO: 55 - DNA, 120 bp, Homo sapiens
  source 1..120: mol_type = genomic DNA
agctatacctgttttatatttttcagtgtttaaaattatttttatatttttatattctgaattttctttctttctttcttttttttttttttttttgagacggagtctcgctgtattgcc

SEQ ID NO: 56 - DNA, 120 bp, Homo sapiens
  source 1..120: mol_type = genomic DNA
ctttttctagatttagtggctgttttgtgtccgaaaggtccacttcgtatgctggttgaaacagctcaggagagaaatgaaacgctttttccagctctcatttactcctgtaagtatttg

SEQ ID NO: 57 - DNA, 120 bp, Homo sapiens
  source 1..120: mol_type = genomic DNA
tcctcctgccttgacttcacaaagtgcttgagttacaggtgtgagctaccacgcctggccatgttttcttgtgtgaaggatctgtttagttttatatctttctgtggctcatatctaatt

SEQ ID NO: 58 - DNA, 120 bp, Homo sapiens
  source 1..120: mol_type = genomic DNA
gtgtctgagagagaaagagatctaataggcaaaaaaataacatcttgttttattttttattgtttgtctaatgctttgggtgattatttgaactttttttcatgtgtttcttagttacag

SEQ ID NO: 59 - DNA, 120 bp, Homo sapiens
  source 1..120: mol_type = genomic DNA
gggaaaatacattctactcaaagtgagtccgtggcaaaagtgtgagtacagagatgggtgaggaattaggaccagactttcagtctgcagcaacattaaacatgtatgttaatttaatac

SEQ ID NO: 60 - DNA, 120 bp, Homo sapiens
  source 1..120: mol_type = genomic DNA
gcctcaccctcccaaagtgctgggattacaggcatgagccaccgcacctggccttcgttttgttttttcaacagaatgtaagaattttttcttacatttttcttacatttttaacagaat

SEQ ID NO: 61 - DNA, 120 bp, Homo sapiens
  source 1..120: mol_type = genomic DNA
caaatatactatttatgataattaacatggggttagcaaactactgcttgaagccaaatatggcccatggcttgtttttgtacagcttataagctaaaaatgctttttacatttaaaaaa

SEQ ID NO: 62 - DNA, 120 bp, Homo sapiens
  source 1..120: mol_type = genomic DNA
aaaaaaattactgtgaccagattggactcagcttgctgctggcatttggttcccaccataacctcatatgtcatgtgtttgcttatatgtacttttgtgttattgttggtgtatcttcag

SEQ ID NO: 63 - DNA, 120 bp, Homo sapiens
  source 1..120: mol_type = genomic DNA
ttgtctttcccaacagcaacaatggtgtggttggtgaatatggcagaaggagacccggaagctcaaaggagagtatccaaaaattccaagtataatgcagaaagtaggtaacttttatta

SEQ ID NO: 64 - DNA, 120 bp, Homo sapiens
  source 1..120: mol_type = genomic DNA
ttgagaatctcttgagaactatgataatgcccagtaaatacacagataagtatttaaggagttcagatactcaaaacccaacaatacagtcaaagcatcctaggttaagacacctcccat

SEQ ID NO: 65 - DNA, 120 bp, Homo sapiens
  source 1..120: mol_type = genomic DNA
tttgctagccagaaacctctgtggctggtggcaccttttcctgagatttgctcaggcccactgggctcattccacctactcagcctagctggctatgcttggctttctctaccagctgga

SEQ ID NO: 66 - DNA, 120 bp, Homo sapiens
  source 1..120: mol_type = genomic DNA
tctgtggaactgggagcttggcctccaggcttcaggctttccctgacttgaggtggtgcttcaccagggaacttcccctttccgcctaggagcctgtctgcctcctgctgccatcaacct

SEQ ID NO: 67 - DNA, 120 bp, Homo sapiens
  source 1..120: mol_type = genomic DNA
ctttgaaatgagaaggctggatgaactgcaggcttcctcagtgagaatgctgctggtattttggggggcacagttctttgtgggactttccctcatattgcaagatatttaacatccctg

SEQ ID NO: 68 - DNA, 120 bp, Homo sapiens
  source 1..120: mol_type = genomic DNA
atgtatggaagttttttaagactatcatgctcaccattttcaatgagattgatagtcattataatgaaatgactggcccataaagacattcactccccgctcctttcccaatccaccctc

SEQ ID NO: 69 - DNA, 120 bp, Homo sapiens
  source 1..120: mol_type = genomic DNA
gcacagaaagggagtcacaagacactgttgcagagaatgatgatggcgggttcagtgaggaatgggaagcccagagggacagtcatctagggcctcatcgctctacacctgagtcacgag

SEQ ID NO: 70 - DNA, 120 bp, Homo sapiens
  source 1..120: mol_type = genomic DNA
gattcctttaggtagctacattatcaacctttttgagaataaaatgaattgagagtgttacagtctaattctatatcacatgtaacttttatttggatatatcagtaatagtgctttttt

SEQ ID NO: 71 - DNA, 120 bp, Homo sapiens
  source 1..120: mol_type = genomic DNA
cagaactttcactgggcatgttcatgactttactgcttggctgagttttccagatatcaaagcccagctgcagcctgtgactttcacactcctggaaaagtagacgtatctgcctgctct

SEQ ID NO: 72 - DNA, 120 bp, Homo sapiens
  source 1..120: mol_type = genomic DNA
attacttggttataaaaagaaacaatctatcattagtacacttaggatgagcttgttactggcatgttggctgtgggaagtatatcttgaaattcactaaaagaccaactattgctgggc

SEQ ID NO: 73 - DNA, 120 bp, Homo sapiens
  source 1..120: mol_type = genomic DNA
cctcccagagtgctgggattacaggtgtgagccactgtgcccggccaaaaaaattttttttaaataccgttaagaagtggaaaataaggccaggtgtggtggctcacacctgtaatccca

SEQ ID NO: 74 - DNA, 120 bp, Homo sapiens
  source 1..120: mol_type = genomic DNA
taaacgggtacaaggtacctgactggggcaatggacatgtggattgtgatgatagttaaattgtacagctctctctatatatagttgtacagctctattaattttccacaatcattgaat

SEQ ID NO: 75 - DNA, 119 bp, Homo sapiens
  source 1..119: mol_type = genomic DNA
gggagtaaaacttggattgggagatttcattttctacagtgttctggttggtaaagcctcagcaacagccagtggagactggaacacaaccatagcctgtttcgtagccatattaattg

SEQ ID NO: 76 - DNA, 120 bp, Homo sapiens
  source 1..120: mol_type = genomic DNA
aattggacctcaccttagtagttctctaccctggccacacattagaatcacttgggagcttttaaaactgtaagctctgccctgagatattcttactcaatttaattgtgtagtttttaa

SEQ ID NO: 77 - DNA, 120 bp, Homo sapiens
  source 1..120: mol_type = genomic DNA
ttggccgggtgaagactttctagtgtttccgtaactagggtaaaaaatgaactctattatatggcttgtacatgtgtttacaagaaagcagtgtactcctcataaataaacttttagttt

SEQ ID NO: 78 - DNA, 120 bp, Homo sapiens
  source 1..120: mol_type = genomic DNA
ggtttgtgccttacattattactccttgccattttcaagaaagcattgccagctcttccaatctccatcacctttgggcttgttttctactttgccacagattatcttgtacagcctttt

SEQ ID NO: 79 - DNA, 120 bp, Homo sapiens
  source 1..120: mol_type = genomic DNA
cagccagtaaggcagctctgtcgtggtagcagatggtcccattattctagggtcttactctttgtatgatgaaaagaatgtgttatgaatcggtgctgtcagccctgctgtcagaccttc

SEQ ID NO: 80 - DNA, 120 bp, Homo sapiens
  source 1..120: mol_type = genomic DNA
tatcattgagaaagcacagctacagcaaagccacctgaatagcaatttgtgattggaagcattcttgagggatccctaatctagagtaatttatttgtgtaaggatcccaaatgtgttgc

SEQ ID NO: 81 - DNA, 120 bp, Homo sapiens
  source 1..120: mol_type = genomic DNA
tttattacttgttatttacgtggcctcagacagtgtatgtattctcgatataacttgtagagtgtgaaatataagtttaactaccaaataaggtctcccagggttagatgactgcgggaa

SEQ ID NO: 82 - DNA, 120 bp, Homo sapiens
  source 1..120: mol_type = genomic DNA
caggactgtcttgtttttggtgtccttggaggtgctggggtgagggtttcagtgggatcatttactctcacatgttgtctgccttctgcttctgtggacactgctttgtacttaattcag

SEQ ID NO: 83 - DNA, 120 bp, Homo sapiens
  source 1..120: mol_type = genomic DNA
aattttatgcagagctcctggtggctctgaagcgccctagggaacagaccaggaacattctcatggtgttagctcacattgggccattagtgctcctttaatgtgagaacaaccgggagg

SEQ ID NO: 84 - DNA, 120 bp, Homo sapiens
  source 1..120: mol_type = genomic DNA
tggcctgggcgggcgtggggcggggcctgggccggcgccgggtccggccgggcgctcagccagctgcgtaaactccgctggagcgcggcggcagagcaggtgagcgggcggtgccggggg

SEQ ID NO: 85 - DNA, 120 bp, Homo sapiens
  source 1..120: mol_type = genomic DNA
gcatttccagcagtgaggagacagccagaagcaagcttttggagctgaaggaacctgagacagaagctagtcccccctctgaattttactgatgaagaaactgaggccacagagctaaag

SEQ ID NO: 86 - DNA, 120 bp, Homo sapiens
  source 1..120: mol_type = genomic DNA
tgccctccctcgttacctcctcatttgtgcctgcccaccttcccagagcctgcgtcttctcagatgcttaacacctgtttagcctctctagttcagagctacaaatttacatgcttgatt

SEQ ID NO: 87 - DNA, 120 bp, Homo sapiens
  source 1..120: mol_type = genomic DNA
ttctggtttgtgggaaaaattatctgttctatcgccccttgatttgggatatcagcctgacccaggggcccaaagagactgggaggacaagagaaaacactttcccaaggacctttccat

SEQ ID NO: 88 - DNA, 120 bp, Homo sapiens
  source 1..120: mol_type = genomic DNA
acacccagcctcattttccattattactgctatgctgattgagcaagtgcactgttaagcactggacacgctgtaagtgatttgttcatcaagacagtcctttgggtaccatgcatatac

SEQ ID NO: 89 - DNA, 120 bp, Homo sapiens
  source 1..120: mol_type = genomic DNA
cgaggacgtgggacttctcagacgtcaggagagtgatgtgagggagctgtgtgaccatagaaagtgacgtgttaaaaaccagcgctgccctctttgaaagccagggagcatcattcattt

SEQ ID NO: 90 - DNA, 120 bp, Homo sapiens
  source 1..120: mol_type = genomic DNA
ctcttcctggacacccagaagaagactcctatgacagttttggggagccttcataccccgaagtctttgagcctcccttgactggctacccaggggaggagctggaggaagaggaggaaa

SEQ ID NO: 91 - DNA, 120 bp, Homo sapiens
  source 1..120: mol_type = genomic DNA
tagatgccacttgtgcttctgaccggctgcaaatttagggctcccatgacccccttaggttcaataacttgctagaatgactcacagaactcaggaaagcactacacttaaaattgcagt

SEQ ID NO: 92 - DNA, 120 bp, Homo sapiens
  source 1..120: mol_type = genomic DNA
agaggccaggtggggtgggctgggctgggcaagagcagctgggccttctgggccagagtttctcttctttttccattctgtgcacgcctcttcagtacgggttactgtctctcctcacac

SEQ ID NO: 93 - DNA, 120 bp, Homo sapiens
  source 1..120: mol_type = genomic DNA
ggggaggatgggcagcccccacacctgcttcccaggggcaggttgcctggcggctctgattcccttggtgccagctgctgagaaccttactgccatttcagttgagcccacctagctctc

SEQ ID NO: 94 - DNA, 120 bp, Homo sapiens
  source 1..120: mol_type = genomic DNA
ctgctgggaagtggcttaatagtaatatcaataaatagatgagtcctgttagaatcttggagtttggtccgttgtaaatgttgacccctctccctgcatcttgggcacccctgggataac

SEQ ID NO: 95 - DNA, 120 bp, Homo sapiens
  source 1..120: mol_type = genomic DNA
gtgcttccagaggcagggctatgctcacattcatggcctctgacagcgaggaagaagtgtgtgatgagcggacgtccctaatgtcggctgagagccccacgccgcgctcctgccaggagg

SEQ ID NO: 96 - DNA, 120 bp, Homo sapiens
  source 1..120: mol_type = genomic DNA
ctggactctttttaaaataaaagtaattcatgtttattctagaaaattagggaaaaaaaattttttttgagatggagtttcactcttgttgcccaggctggagtgcgatggtatgatctc

SEQ ID NO: 97 - DNA, 120 bp, Homo sapiens
  source 1..120: mol_type = genomic DNA
agaagccaggagaacgaggaggacggtgaggaggaccctgaccgctatgtctgtagtggggttcccgggcggccgccaggcctggaggaagagctgaccctcaaatacggagcgaagcac

SEQ ID NO: 98 - DNA, 120 bp, Homo sapiens
  source 1..120: mol_type = genomic DNA
gacattgtcctatggatatgtcctttcatttgataatatgtttacgtggccatagtgcctggggctgggccgggaatggaaacttgatctctggggcctggcctttgaagccagttcatg

SEQ ID NO: 99 - DNA, 120 bp, Homo sapiens
  source 1..120: mol_type = genomic DNA
cctctctgcggccccaagtgttcgtggtaagtgcagtgactcccaacctgcttttgaaccctctttttccattaggattttctccgtggaggcagatttccatgggagtttgctgtggca

SEQ ID NO: 100 - DNA, 120 bp, Homo sapiens
  source 1..120: mol_type = genomic DNA
catctacacgccattcactgaggacacaccctcggtgggccagcgcctcctcaactccgtgctgaacaccctcatcatgatcagcgtcatcgtggttatgaccatcttcttggtggtgct

SEQ ID NO: 101 - DNA, 120 bp, Homo sapiens
  source 1..120: mol_type = genomic DNA
atctttaatgttttgtaatatttccgatcttttatatacatacattttataaggacattcagatgatcaggttcgtaaagttttatgttcggttaaatttaacagcgtgtcattgttcag

SEQ ID NO: 102 - DNA, 120 bp, Homo sapiens
  source 1..120: mol_type = genomic DNA
acagcttcaggggaggccttggaggaatttttgaaagtggaatgagggaagcagcctgctcaagggaacacttgttttctggtgaggaggccgcatgtatgaatgacgtttgtgggttag

SEQ ID NO: 103 - DNA, 120 bp, Homo sapiens
  source 1..120: mol_type = genomic DNA
gtgatcgtgcaatttctgttgtctagttcatccatggctggttgatcatgtcttcactgatgctgctgttcctcttcacctatatctaccttgggtaagtgacagataagcagcagggtc

SEQ ID NO: 104 - DNA, 120 bp, Homo sapiens
  source 1..120: mol_type = genomic DNA
ctgtcatactagaaaaggaggtattaaaagagccacctgtacagcctcgtatctcatccagcacactgctgcagatggaatattatgatttagcttgagaaaatgcagcaactctttgtt

SEQ ID NO: 105 - DNA, 120 bp, Homo sapiens
  source 1..120: mol_type = genomic DNA
ggaagtgctcaagacctacaatgtggccatggactaccccaccctcttgctgactgtctggaacttcggggcagtgggcatggtgtgcatccactggaagggccctctggtgctgcagca

SEQ ID NO: 106 - DNA, 120 bp, Homo sapiens
  source 1..120: mol_type = genomic DNA
ctaaacagaaggtgctgcaggctggggagtttgttcatgttgaaatccctgtgatggaggtgagcagaggcagtctctgcctgtgcctcttatttggggatgaagttaaagtccctgtag

SEQ ID NO: 107 - DNA, 120 bp, Homo sapiens
  source 1..120: mol_type = genomic DNA
gtcttcctagatctcgtggctgtgctgtgtcccaaagggcctctgagaatgctggtagaaactgcccaggagagaaatgagcccatattccctgccctgatatactcatgtgagtgagcc

SEQ ID NO: 108 - DNA, 120 bp, Homo sapiens
  source 1..120: mol_type = genomic DNA
cagacagcttccccattttatcccatagacactcaaagggtgaaattcatggtctttcccgagactctcttctccggtcttccctgtcttagtccccacctggctgccattctagactgt

SEQ ID NO: 109 - DNA, 120 bp, Homo sapiens
  source 1..120: mol_type = genomic DNA
ccttttctcctcccccagctgccatggtgtggacggttggcatggcgaagctggacccctcctctcagggtgccctccagctcccctacgacccggagatgggtgagtatcttggggagc

SEQ ID NO: 110 - DNA, 120 bp, Homo sapiens
  source 1..120: mol_type = genomic DNA
gacacccagaagaagactcctatgacagttttggggagccttcataccccgaagtctttgagcctcccttgactggctacccaggggaggagctggaggaagaggaggaaagtaaggtgc

SEQ ID NO: 111 - DNA, 120 bp, Homo sapiens
  source 1..120: mol_type = genomic DNA
ttactgggagaattaagtgagttaatatgagtgaagggcttagaagagtgtctactgcacgtgagtgctcaggcaagctggatcctgctgcagaaagcaagctcttgatcctgggcatgg

SEQ ID NO: 112 - DNA, 120 bp, Homo sapiens
  source 1..120: mol_type = genomic DNA
ttaatccctgtgggggtggggaaagcagcagggaggtcatctagccctcgtcctcactgctgcactgggcccagttggcaggctgagagccacaggtctgtggtcagggtgccaggaaat

SEQ ID NO: 113 - DNA, 120 bp, Homo sapiens
  source 1..120: mol_type = genomic DNA
ggggcgtgaagcttggcctcggggacttcatcttctacagtgtgctggtgggcaaggcggctgccacgggcagcggggactggaataccacgctggcctgcttcgtggccatcctcattg

SEQ ID NO: 114 - DNA, 120 bp, Homo sapiens
  source 1..120: mol_type = genomic DNA
tttagttttccttttccttttcgggcaaggatgtgcagaggccatgctgaggtcttgcagccctgggagacttttgggttgtagctgcctatagctgccgagtagccccagggagtagtg

SEQ ID NO: 115 - DNA, 120 bp, Homo sapiens
  source 1..120: mol_type = genomic DNA
ggcttgtgtctgaccctcctgctgcttgctgtgttcaagaaggcgctgcccgccctccccatctccatcacgttcgggctcatcttttacttctccacggacaacctggtgcggccgttc

SEQ ID NO: 116 - DNA, 120 bp, Homo sapiens
  source 1..120: mol_type = other DNA
ggaaacaaaacagcggctggtctggaaggaacctgagctacgagccgcggcggcagcggggcggcggggaagcgtatacctaatctgggagcctgcaagtgacaacagcctttgcggtcc

SEQ ID NO: 117 - DNA, 120 bp, Homo sapiens
  source 1..120: mol_type = other DNA
ccttagacagcttggcctggaggagaacacatgaaagaaagaacctcaagaggctttgttttctgtgaaacagtatttctatacagttgctccaatgacagagttacctgcaccgttgtc

SEQ ID NO: 118 - DNA, 120 bp, Homo sapiens
  source 1..120: mol_type = other DNA
tcctacttccagaatgcacagatgtctgaggacaaccacctgagcaatactgtacgtagccagaatgacaatagagaacggcaggagcacaacgacagacggagccttggccaccctgag

SEQ ID NO: 119 - DNA, 120 bp, Homo sapiens
  source 1..120: mol_type = other DNA
agccattatctaatggacgaccccagggtaactcccggcaggtggtggagcaagatgaggaagaagatgaggagctgacattgaaatatggcgccaagcatgtgatcatgctctttgtcc

SEQ ID NO: 120 - DNA, 120 bp, Homo sapiens
  source 1..120: mol_type = other DNA
ccctgtgactctctgcatggtggtggtcgtggctaccattaagtcagtcagcttttatacccggaaggatgggcagctaatctataccccattcacagaagataccgagactgtgggcca

SEQ ID NO: 121 - DNA, 120 bp, Homo sapiens
  source 1..120: mol_type = other DNA
cagagagccctgcactcaattctgaatgctgccatcatgatcagtgtcattgttgtcatgactatcctcctggtggttctgtataaatacaggtgctataaggtcatccatgcctggctt

SEQ ID NO: 122 - DNA, 120 bp, Homo sapiens
  source 1..120: mol_type = other DNA
ttattatatcatctctattgttgctgttctttttttcattcatttacttgggggaagtgtttaaaacctataacgttgctgtggactacattactgttgcactcctgatctggaattttg

SEQ ID NO: 123 - DNA, 120 bp, Homo sapiens
  source 1..120: mol_type = other DNA
tggtgtggtgggaatgatttccattcactggaaaggtccacttcgactccagcaggcatatctcattatgattagtgccctcatggccctggtgtttatcaagtacctccctgaatggac

SEQ ID NO: 124 - DNA, 120 bp, Homo sapiens
  source 1..120: mol_type = other DNA
actgcgtggctcatcttggctgtgatttcagtatatgatttagtggctgttttgtgtccgaaaggtccacttcgtatgctggttgaaacagctcaggagagaaatgaaacgctttttcca

SEQ ID NO: 125 - DNA, 120 bp, Homo sapiens
  source 1..120: mol_type = other DNA
cagctctcatttactcctcaacaatggtgtggttggtgaatatggcagaaggagacccggaagctcaaaggagagtatccaaaaattccaagtataatgcagaaagcacagaaagggagt

SEQ ID NO: 126 - DNA, 120 bp, Homo sapiens
  source 1..120: mol_type = other DNA
gtcacaagacactgttgcagagaatgatgatggcgggttcagtgaggaatgggaagcccagagggacagtcatctagggcctcatcgctctacacctgagtcacgagctgctgtccagga

SEQ ID NO: 127 - DNA, 120 bp, Homo sapiens
  source 1..120: mol_type = other DNA
gaactttccagcagtatcctcgctggtgaagacccagaggaaaggggagtaaaacttggattgggagatttcattttctacagtgttctggttggtaaagcctcagcaacagccagtgga

SEQ ID NO: 128 - DNA, 120 bp, Homo sapiens
  source 1..120: mol_type = other DNA
gagactggaacacaaccatagcctgtttcgtagccatattaattggtttgtgccttacattattactccttgccattttcaagaaagcattgccagctcttccaatctccatcacctttg

SEQ ID NO: 129 - DNA, 120 bp, Homo sapiens
  source 1..120: mol_type = other DNA
tgggcttgttttctactttgccacagattatcttgtacagccttttatggaccaattagcattccatcaattttatatctagcatatttgcggttagaatcccatggatgtttcttcttt

SEQ ID NO: 130 - DNA, 120 bp, Homo sapiens
  source 1..120: mol_type = other DNA
ttgactataacaaaatctggggaggacaaaggtgattttcctgtgtccacatctaacaaagtcaagattcccggctggacttttgcagcttccttccaagtcttcctgaccaccttgcac

SEQ ID NO: 131 - DNA, 120 bp, Homo sapiens
  source 1..120: mol_type = other DNA
actattggactttggaaggaggtgcctatagaaaacgattttgaacatacttcatcgcagtggactgtgtccctcggtgcagaaactaccagatttgagggacgaggtcaaggagatatg

SEQ ID NO: 132 - DNA, 120 bp, Homo sapiens
  source 1..120: mol_type = other DNA
tgataggcccggaagttgctgtgccccatcagcagcttgacgcgtggtcacaggacgatttcactgacactgcgaactctcaggactaccgttaccaagaggttaggtgaagtggtttaa

SEQ ID NO: 133 - DNA, 120 bp, Homo sapiens
  source 1..120: mol_type = other DNA
aaaccaaacggaactcttcatcttaaactacacgttgaaaatcaacccaataattctgtattaactgaattctgaacttttcaggaggtactgtgaggaagagcaggcaccagcagcaga

SEQ ID NO: 134 - DNA, 120 bp, Homo sapiens
  source 1..120: mol_type = other DNA
gaatggggaatggagaggtgggcaggggttccagcttccctttgattttttgctgcagactcatcctttttaaatgagacttgttttcccctctctttgagtcaagtcaaatatgtagat

SEQ ID NO: 135 - DNA, 120 bp, Homo sapiens
  source 1..120: mol_type = other DNA
attgcctttggcaattcttcttctcaagcactgacactcattaccgtctgtgattgccatttcttcccaaggccagtctgaacctgaggttgctttatcctaaaagttttaacctcaggt

SEQ ID NO: 136 - DNA, 120 bp, Homo sapiens
  source 1..120: mol_type = other DNA
gttccaaattcagtaaattttggaaacagtacagctatttctcatcaattctctatcatgttgaagtcaaatttggattttccaccaaattctgaatttgtagacatacttgtacgctca

SEQ ID NO: 137 - DNA, 120 bp, Homo sapiens
  source 1..120: mol_type = other DNA
cacttgccccagatgcctcctctgtcctcattcttctctcccacacaagcagtctttttctacagccagtaaggcagctctgtcgtggtagcagatggtcccattattctagggtcttac

SEQ ID NO: 138 - DNA, 120 bp, Homo sapiens
  source 1..120: mol_type = other DNA
actctttgtatgatgaaaagaatgtgttatgaatcggtgctgtcagccctgctgtcagaccttcttccacagcaaatgagatgtatgcccaaagacggtagaattaaagaagagtaaaat

SEQ ID NO: 139 - DNA, 120 bp, Homo sapiens
  source 1..120: mol_type = other DNA
atggctgttgaagcactttctgtcctggtattttgtttttgcttttgccacacagtagctcagaatttgaacaaatagccaaaagctggtggttgatgaattatgaactagttgtatcaa

SEQ ID NO: 140 - DNA, 120 bp, Homo sapiens
  source 1..120: mol_type = other DNA
aacacaaagcaagagttggggaaagccatatttaacttggtgagctgtgggagaacctggtggcagaaggagaaccaactgccaaggggaaagagaaggggcctccagcagcgaagggga

SEQ ID NO: 141 - DNA, 120 bp, Homo sapiens
  source 1..120: mol_type = other DNA
gatacagtgagctaatgatgtcaaggaggagtttcaggttattctcgtcagctccacaaatgggtgctttgtggtctctgcccgcgttacctttcctctcaatgtacctttgtgtgaact

SEQ ID NO: 142 - DNA, 120 bp, Homo sapiens
  source 1..120: mol_type = other DNA
ctgggcagtggaggtgcctgctgcagttaccatggagttcaggctctgggcagctcagtcaggcaaaacacacaaacagccatcagcctgtgtgggctcagggcacctctggacaaaggc

SEQ ID NO: 143 - DNA, 120 bp, Homo sapiens
  source 1..120: mol_type = other DNA
gcttgtggggcataaccttctttaccacagagagcccttagctatgctgatcagaccgtaagcgtttatgagaaacttagtttcctcctgtggctgaggaggggccagctttttcttctt

SEQ ID NO: 144 - DNA, 120 bp, Homo sapiens
  source 1..120: mol_type = other DNA
ttttgcctgctgttttctctcccaatctatgatatgatatgacctggtttggggctgtctttggtgtttagaatatttgttttctgtcccaggatatttcttataagaacctaacttcaa

SEQ ID NO: 145 - DNA, 120 bp, Homo sapiens
  source 1..120: mol_type = other DNA
aagagtagtgtgcgagtactgatctgaatttaaattaaaattggcttatattaggcagtcacagacaggaaaaataagagctatgcaaagaaagggggatttaaagtagtaggttctatc

SEQ ID NO: 146 - DNA, 120 bp, Homo sapiens
  source 1..120: mol_type = other DNA
tcatctcaattcatttttttccatgaaatcccttcttccaagattcattccctctctcagacatgtgctagcatgggtattatcattgagaaagcacagctacagcaaagccacctgaat

SEQ ID NO: 147 - DNA, 120 bp, Homo sapiens
  source 1..120: mol_type = other DNA
atagcaatttgtgattggaagcattcttgagggatccctaatctagagtaatttatttgtgtaaggatcccaaatgtgttgcacctttcatgatacatttcttctctgaagagggtacgt

SEQ ID NO: 148 - DNA, 120 bp, Homo sapiens
  source 1..120: mol_type = other DNA
gtggggtgtgtgtatttaaatccatcctatgtattactgattgtcctgtgtagaaagatggcaattattctgtctctttctccaagtttgagccacatctcagccacattgttagacagt

SEQ ID NO: 149 - DNA, 120 bp, Homo sapiens
  source 1..120: mol_type = other DNA
gtgtacagagaacctatctttcctttttttttttttaaaggacaggattttgctgtgttgcccaggctagacttgaactcctgggctcaagtaatccacctcagcctgagtagctgagac

SEQ ID NO: 150 - DNA, 120 bp, Homo sapiens
  source 1..120: mol_type = other DNA
actacagcccatcttatttctttaaatcattcatctcaggcagagaacttttccctcaaacattctttttagaattagttcagtcattcctaaaacatccaaatgctagtcttccaccat

SEQ ID NO: 151 - DNA, 120 bp, Homo sapiens
  source 1..120: mol_type = other DNA
atgaaaaatagattgtcactggaaagaacagtagcaatttccataaggatgtgccttcactcacacgggacaggcggtggttatagagtcgggcaaaaccagcagtagagtatgaccagc

SEQ ID NO: 152 - DNA, 120 bp, Homo sapiens
  source 1..120: mol_type = other DNA
gccaagccaatctgcttaataaaaagatggaagacagtaaggaaggaaagtagccactaagagtctgagtctgactgggctacagaataaagggtatttatggacagaatgtcattacat

SEQ ID NO: 153 - DNA, 120 bp, Homo sapiens
  source 1..120: mol_type = other DNA
atgcctatgggaataccaatcatatttggaagatttgcagattttttttcagagaggaaagactcaccttcctgtttttggttctcagtaggttcgtgtgtgttcctagaatcacagctc

SEQ ID NO: 154 - DNA, 120 bp, Homo sapiens
  source 1..120: mol_type = other DNA
tctgactccaaatgactcaatttctcaattagaaaaagtagaagctttctaagcaacttggaagaaaacagtcataagtaagcaatttgttgattttactacagaagcaacaactgaaga

SEQ ID NO: 155 - DNA, 120 bp, Homo sapiens
  source 1..120: mol_type = other DNA
gaggcagtgtttttactttcagactccgggattcccattctgtagtctctctgcttttaaaaaccctccttttgcaatagatgcccaaacagatgatgtttattacttgttatttacgtg

SEQ ID NO: 156 - DNA, 120 bp, Homo sapiens
  source 1..120: mol_type = other DNA
tggcctcagacagtgtatgtattctcgatataacttgtagagtgtgaaatataagtttaactaccaaataaggtctcccagggttagatgactgcgggaagcctttgatcccaaccccca

SEQ ID NO: 157 - DNA, 120 bp, Homo sapiens
  source 1..120: mol_type = other DNA
caaggctttgtatatttgatcatttgtgatctaaccctggaagaaaaagagctcagaaaccactatgaaaaaatttgttcagtgttttctgtgttcccgtaggttctggagtctgaggat

SEQ ID NO: 158 - DNA, 120 bp, Homo sapiens
  source 1..120: mol_type = other DNA
atgcaaagatgaataagataaattctcagaatgtagttataatctcttgttttctggtatatgccatctttctttaacttctctaaaatattgggtatttgtcaaataaccacttttaac

SEQ ID NO: 159 - DNA, 120 bp, Homo sapiens
  source 1..120: mol_type = other DNA
acagttaccattactgagggcttatacattggtgttataaaagtgacttgattcagaaatcaatccattcagtaaagtactccttctctaaatttgctgttatgtctataaggaacagtt

SEQ ID NO: 160 - DNA, 120 bp, Homo sapiens
  source 1..120: mol_type = other DNA
tttgacctgcccttctcctcacctcctcacctgccttccaacattgaatttggaaggagacgtgaaaattggacatttggttttgcccttgggctggaaactatcatataatcataagtt

SEQ ID NO: 161 - DNA, 120 bp, Homo sapiens
  source 1..120: mol_type = other DNA
tttgagcctagaagtgatccttgtgatcttctcacctctttaaattcccacaacacaagagattaaaaacagaggtttcagctcttcatagtgcgttgtgaaatggctggccagagtgta

SEQ ID NO: 162 - DNA, 120 bp, Homo sapiens
  source 1..120: mol_type = other DNA
taccaacaaagctgtcatcgggctcacagctcagagacatctgcatgtgatcatctgcatagtcctctcctctaacgggaaacacctcagatttgcatataaaaaagcaccctggtgctg

SEQ ID NO: 163 - DNA, 120 bp, Homo sapiens
  source 1..120: mol_type = other DNA
tgaaatgaacccctttcttgaacatcaaagctgtctcccacagccttgggcagcagggtgcctcttagtggatgtgctgggtccaccctgagccctgacatgtggtggcagcattgccag

SEQ ID NO: 164 - DNA, 120 bp, Homo sapiens
  source 1..120: mol_type = other DNA
agttggtctgtgtgtctgtgtagcagggacgatttcccagaaagcaattttccttttgaaatacgtaattgttgagactaggcagtttcaaagtcagctgcatatagtagcaagtacagg

SEQ ID NO: 165 - DNA, 120 bp, Homo sapiens
  source 1..120: mol_type = other DNA
ggactgtcttgtttttggtgtccttggaggtgctggggtgagggtttcagtgggatcatttactctcacatgttgtctgccttctgcttctgtggacactgctttgtacttaattcagac

SEQ ID NO: 166 - DNA, 120 bp, Homo sapiens
  source 1..120: mol_type = other DNA
tcctacttccagaatgcacagatgtctgaggacaaccacctgagcaatactaatgacaatagagaacggcaggagcacaacgacagacggagccttggccaccctgagccattatctaat

SEQ ID NO: 167 - DNA, 120 bp, Homo sapiens
  source 1..120: mol_type = other DNA
gcgtaaactccgctggagcgcggcggcagagcaggcatttccagcagtgaggagacagccagaagcaagcttttggagctgaaggaacctgagacagaagctagtcccccctctgaattt

SEQ ID NO: 168 - DNA, 120 bp, Homo sapiens
  source 1..120: mol_type = other DNA
tttactgatgaagaaactgaggccacagagctaaagtgacttttcccaaggtcgcccagcgaggacgtgggacttctcagacgtcaggagagtgatgtgagggagctgtgtgaccataga

SEQ ID NO: 169 - DNA, 120 bp, Homo sapiens
  source 1..120: mol_type = other DNA
gaaagtgacgtgttaaaaaccagcgctgccctctttgaaagccagggagcatcattcatttagcctgctgagaagaagaaaccaagtgtccgggattcagacctctctgcggccccaagt

SEQ ID NO: 170 - DNA, 120 bp, Homo sapiens
  source 1..120: mol_type = other DNA
gtgttcgtggtgcttccagaggcagggctatgctcacattcatggcctctgacagcgaggaagaagtgtgtgatgagcggacgtccctaatgtcggctgagagccccacgccgcgctcct

SEQ ID NO: 171 - DNA, 120 bp, Homo sapiens
  source 1..120: mol_type = other DNA
ctgccaggagggcaggcagggcccagaggatggagagaacactgcccagtggagaagccaggagaacgaggaggacggtgaggaggaccctgaccgctatgtctgtagtggggttcccgg

SEQ ID NO: 172 - DNA, 120 bp, Homo sapiens
  source 1..120: mol_type = other DNA
gggcggccgccaggcctggaggaagagctgaccctcaaatacggagcgaagcacgtgatcatgctgtttgtgcctgtcactctgtgcatgatcgtggtggtagccaccatcaagtctgtg

SEQ ID NO: 173 - DNA, 120 bp, Homo sapiens
  source 1..120: mol_type = other DNA
tgcgcttctacacagagaagaatggacagctcatctacacgccattcactgaggacacaccctcggtgggccagcgcctcctcaactccgtgctgaacaccctcatcatgatcagcgtca

SEQ ID NO: 174 - DNA, 120 bp, Homo sapiens
  source 1..120: mol_type = other DNA
catcgtggttatgaccatcttcttggtggtgctctacaagtaccgctgctacaagttcatccatggctggttgatcatgtcttcactgatgctgctgttcctcttcacctatatctacct

SEQ ID NO: 175 - DNA, 120 bp, Homo sapiens
  source 1..120: mol_type = other DNA
cttggggaagtgctcaagacctacaatgtggccatggactaccccaccctcttgctgactgtctggaacttcggggcagtgggcatggtgtgcatccactggaagggccctctggtgctg

SEQ ID NO: 176 - DNA, 120 bp, Homo sapiens
  source 1..120: mol_type = other DNA
tgcagcaggcctacctcatcatgatcagtgcgctcatggccctagtgttcatcaagtacctcccagagtggtccgcgtgggtcatcctgggcgccatctctgtgtatgatctcgtggctg

SEQ ID NO: 177 - DNA, 120 bp, Homo sapiens
  source 1..120: mol_type = other DNA
tgtgctgtgtcccaaagggcctctgagaatgctggtagaaactgcccaggagagaaatgagcccatattccctgccctgatatactcatctgccatggtgtggacggttggcatggcgaa

SEQ ID NO: 178 - DNA, 120 bp, Homo sapiens
  source 1..120: mol_type = other DNA
aagctggacccctcctctcagggtgccctccagctcccctacgacccggagatggaagaagactcctatgacagttttggggagccttcataccccgaagtctttgagcctcccttgact

SEQ ID NO: 179 - DNA, 120 bp, Homo sapiens
  source 1..120: mol_type = other DNA
ctggctacccaggggaggagctggaggaagaggaggaaaggggcgtgaagcttggcctcggggacttcatcttctacagtgtgctggtgggcaaggcggctgccacgggcagcggggact

SEQ ID NO: 180 - DNA, 120 bp, Homo sapiens
  source 1..120: mol_type = other DNA
ctggaataccacgctggcctgcttcgtggccatcctcattggcttgtgtctgaccctcctgctgcttgctgtgttcaagaaggcgctgcccgccctccccatctccatcacgttcgggct

SEQ ID NO: 181 - DNA, 120 bp, Homo sapiens
  source 1..120: mol_type = other DNA
ctcatcttttacttctccacggacaacctggtgcggccgttcatggacaccctggcctcccatcagctctacatctgagggacatggtgtgccacaggctgcaagctgcagggaattttc

SEQ ID NO: 182 - DNA, 120 bp, Homo sapiens
  source 1..120: mol_type = other DNA
tcattggatgcagttgtatagttttacactctagtgccatatatttttaagacttttctttccttaaaaaataaagtacgtgtttacttggtgaggaggaggcagaaccagctctttggt

SEQ ID NO: 183 - DNA, 120 bp, Homo sapiens
  source 1..120: mol_type = other DNA
gtgccagctgtttcatcaccagactttggctcccgctttggggagcgcctcgcttcacggacaggaagcacagcaggtttatccagatgaactgagaaggtcagattagggcggggagaa

SEQ ID NO: 184 - DNA, 120 bp, Homo sapiens
  source 1..120: mol_type = other DNA
aagagcatccggcatgagggctgagatgcgcaaagagtgtgctcgggagtggcccctggcacctgggtgctctggctggagaggaaaagccagttccctacgaggagtgttcccaatgct

SEQ ID NO: 185 - DNA, 120 bp, Homo sapiens
  source 1..120: mol_type = other DNA
ctttgtccatgatgtccttgttattttattgcctttagaaactgagtcctgttcttgttacggcagtcacactgctgggaagtggcttaatagtaatatcaataaatagatgagtcctgt

SEQ ID NO: 186 - DNA, 120 bp, Homo sapiens
  source 1..120: mol_type = other DNA
ggggcctgggccggcgccgggtccggccgggcgctcagccagctgcgtaaactccgctggagcgcggcggcagagcaggcatttccagcagtgaggagacagccagaagcaagcttttgg

SEQ ID NO: 187 - protein, 4 aa, Homo sapiens
VRSQ

SEQ ID NO: 188 - RNA, 10 nt, Homo sapiens
  source 1..10: mol_type = mRNA
ctgctacaag

SEQ ID NO: 189 - DNA, 120 bp, Homo sapiens
  source 1..120: mol_type = genomic DNA
aaatgacgacaacggtgagggttctcgggcggggcctgggacaggcagctccggggtccgcggtttcacatcggaaacaaaacagcggctggtctggaaggaacctgagctacgagccgc

SEQ ID NO: 190 - DNA, 120 bp, Homo sapiens
  source 1..120: mol_type = genomic DNA
aaatgctgtttgctcgaagacgtctcagggcgcaggtgccttgggccgggattagtagccgtctgaactggagtggagtaggagaaagaggaagcgtcttgggctgggtctgcttgagca

SEQ ID NO: 191 - DNA, 24 bp, synthetic construct
  source 1..24: mol_type = other DNA
tcacctatatctaccttggggaag

SEQ ID NO: 192 - DNA, 24 bp, synthetic construct
  source 1..24: mol_type = other DNA
ccagcacactgtagaagatgaagt